(19) United States
(12) Patent Application Publication
(10) Pub. No.: US 2026/0062447 A1
(43) Pub. Date: Mar. 5, 2026

(54) COMPOUNDS

(71) Applicant: UNIVERSITY OF SOUTHAMPTON, Southampton, Hampshire (GB)

(72) Inventors: Ali TAVASSOLI, Southampton, Hampshire (GB); Reece GARDNER, Southampton, Hampshire (GB)

(21) Appl. No.: 19/104,374

(22) PCT Filed: Aug. 21, 2023
(86) PCT No.: PCT/GB2023/052178

§ 371 Date: Feb. 18, 2025

(30) Foreign Application Priority Data
Aug. 22, 2022 (GB) 2212184.2

(51) Int. Cl. C07K 7/64; A61K 38/00; A61K 45/06; A61K 49/00

(52) U.S. Cl. CPC C07K 7/64; A61K 45/06; A61K 49/0039; A61K 38/00

(57) ABSTRACT

The invention provides cyclic peptides that are able to disrupt the typical response to hypoxia and which have particular utility in the treatment of cancers and von Hippel-Lindau disease.

### FIELD OF THE INVENTION

The invention is in the field of therapeutic agents suitable for use in treating diseases or conditions that involve the response to hypoxia.

### BACKGROUND

Hypoxia is a state of reduced oxygen concentration that can arise under normal conditions such as embryonic development, but also plays a key role in multiple pathological conditions, such as cardiac arrest, stroke and cancer.¹ Hypoxia has particular relevance in cancers as solid tumours contain hypoxic regions (pO₂≤2.5 mmHg)² that occur due to tumour cell growth exceeding the capacity of the surrounding vascular infrastructure. Hypoxia inducible factors (HIF) are heterodimeric transcription factors that assemble in hypoxia and reprogram gene expression to allow survival and growth of cells in a low oxygen microenvironment.³⁻⁵ The expression of several hundred genes has been directly linked to HIF-1 activation, and genomic analysis of HRE sequences estimates that HIF-1 mediates the expression of up to 1% of the genome.^{6,7} Whilst HIF activity impacts a diverse set of cellular pathways, the primary means by which hypoxic response is enacted is through the reprogramming of glucose metabolism, and the promotion of angiogenesis and proliferation. This response is believed to promote an aggressive phenotype and prolong tumour survival. As such, HIFs has long been proposed to be an attractive target for cancer therapy.

HIF is a heterodimeric transcription factor, which comprises of an oxygen-sensitive a subunit, and a constitutively-expressed P subunit (also known as the aryl hydrocarbon nuclear receptor translocator, ARNT). There are 3 isoforms of the HIF-α that bind to HIF-1β, with HIF-1α and HIF-2α being responsible for orchestrating hypoxia-response. The α-subunit of HIF is continually expressed but subject to post-translational modifications by oxygen-dependent proline hydroxylases (PHD). The hydroxylation of two prolines (P402 and P564 in HIF-1α) enables recognition by the von Hippel-Lindau protein and its associated E3 ligase complex, which triggers rapid ubiquitination and proteasomal degradation. Thus, HIF activity is acutely oxygen-sensitive, with HIF-1α having a half-life of less than 5 minutes in normoxia. HIF-1α is not degraded in hypoxia due to the absence of the molecular oxygen required for prolyl hydroxylation. The subsequent increase in HIF-α concentration causes it to translocate to the nucleus where it forms a dimeric complex with the constitutively expressed HIF-S to form the active HIF transcription factor, HIF binds to numerous hypoxia-response elements (HRE) present in the genome to reprogramme hypoxic cells to allow their survival and growth. The protein-protein interaction of the α and β subunit of HIF is a potential key point of therapeutic intervention, with recent successes in disrupting this interaction in HIF-2 translating into a potential cancer treatment programme.

The interplay between these isoforms is intricate and whilst there is considerable overlap in the genes regulated by HIF-1α and HIF-2α they have also been shown to perform distinct roles in the response to hypoxia.

This has led to both isoforms becoming prominent therapeutic targets, and whilst there has been some success in targeting HIF-2a, HIF-ta still proves to be an intractable target.

There is therefore a need for inhibitors that are able to target HIF-1α and disrupt the role HIF-1α has in the response to hypoxia.

### SUMMARY OF THE INVENTION

The present invention provides a series of cyclic peptides that bind to HIF-ta and inhibit dimerisation of HIF-1β and HIF-1α and the subsequent hypoxia-response signaling in cells.

### DETAILED DESCRIPTION OF THE INVENTION

### Definitions

As used herein, “alkyl” means a linear or branched alkane missing at least one hydrogen such that a bonding position is available, i.e. an alkyl group. Where a carbon chain length is not specified herein, “alkyl” means a C₁-C₁₀ alkyl group. In some embodiments, “alkyl” means a C₄-C₆ alkyl group. In other embodiments, “alkyl” means a C₁-C₃ alkyl group. Examples include methyl, ethyl, n-propyl and t-butyl. It may be monovalent, e.g. propyl, or divalent, e.g. propylene. A monovalent alkyl group may also be described by —CₙH₂ₙ₊₁ and a divalent alkyl group may also be described by —(CH₂)ₙ—, where n is independently selected from 1 to 10 for each substituent if not specified herein.

As used herein, “O-alkyl” means an alkyl group as defined above bonded to an oxygen atom, where said oxygen atom has a further available bonding position to form, for example, an ether via a C—O—C bond.

As used herein “halogen” or “halo” means an element from group 17 of the periodic table, preferably selected from fluorine, chlorine, bromine, and iodine.

As used herein “haloalkyl” means an alkyl group as defined above, which may be substituted with up to 10 halogen atoms or more preferably up to 5 halogens. For example, they may be substituted by 1, 2, 3, 4 or 5 halogen atoms. Preferably, the halogen is fluorine. Preferably the haloalkyl is selected from —CF₃, —CHF₂, and —CH₂F, further preferably —CF₃.

As used herein “aryl” or “aromatic group” means a monocyclic, bicyclic or tricyclic monovalent, divalent, trivalent or tetravalent (as appropriate) aromatic radical, such as phenyl, biphenyl, naphthyl, anthracenyl, which can be optionally substituted by preferably up to three substituents selected from the group comprising or consisting of halogen, C₁-C₃ alkyl, C₁-C₃ haloalkyl, NO₂, CN, OH or O—(C₁-C₃ alkyl). As used herein “aryl” or “aromatic group” includes aromatic heterocycles (i.e. cyclic compounds that has both carbon and non-carbon atoms forming the ring structure). Preferred non-carbon atoms (i.e. “heteroatoms”) are nitrogen, oxygen and sulphur. Preferably heterocycles contain one or two heteroatoms, preferably one. When there is more than one heteroatom in a heterocycle, the heteroatoms may be the same atom or different atoms. Examples of suitable aromatic heterocyclic rings containing one or more heteroatoms selected from O, S and N include furan, thiophene, pyrrole, imidazole, pyrazole, isoxazole, thiazole, isothiazole, pyridine, pyran, thiopyran, diazine, oxazine, thiazine, dioxine and dithiin.

Where an atom is identified herein, whether written or structurally indicated, said atom may be replaced by any known atomic isotopes of said atom, including stable and radioactive isotopes (i.e. variants of said atom differing in neutron number); for example, a deuterium atom may replace a hydrogen atom where a hydrogen atom is indicated. Synthetic methods for incorporating stable- and radio-isotopes are well-known in the art. Preferably, the atom is as identified herein.

As used herein, the above groups can be followed by the suffix -ene. This means that the group is divalent, i.e. a linker group. The linker (i.e. divalent) groups listed herein or in the claims are not ‘direction specific’. They can be reversed.

Compounds with which the invention is concerned, which may exist in one or more stereoisomeric form because of the presence of asymmetric atoms or rotational restrictions, can exist as a number of stereoisomers with R or S stereochemistry at each chiral centre or as atropisomers with R or S stereochemistry at each chiral axis.

The invention includes all such enantiomers and diastereoisomers and mixtures thereof.

Where a chemical structure is shown, the accuracy of the structure takes preference over the compound name.

The full and abbreviated names of the amino acids or unnatural amino acids described herein will be known to the skilled person, however for the avoidance of doubt the below nomenclature is used herein:

| Abbreviation | Name | Structure |
|---|---|---|
| cys | cysteine | 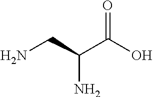 |
| leu | leucine | 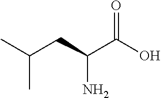 |
| phe | phenylalanine | 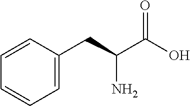 [SMILES: N[C@@H](Cc1ccccc1)C(=O)O] |
| val | valine | 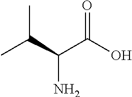 |
| tyr | tyrosine | 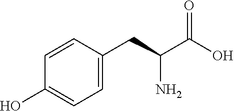 [SMILES: N[C@@H](Cc1ccc(O)cc1)C(=O)O] |
| tyr(Me) | O-methyltyrosine | 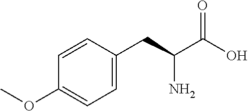 |
| phg | phenylglycine | 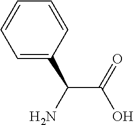 [SMILES: N[C@H](C(=O)O)c1ccccc1] |
| 4-Pal | 4-pyridylalanine | 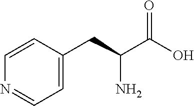 [SMILES: N[C@@H](Cc1ccncc1)C(=O)O] |
| phe(4-NO₂) | 4-nitrophenylalanine | 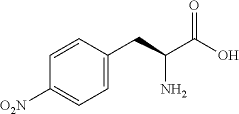 [SMILES: N[C@@H](Cc1ccc([N+](=O)[O-])cc1)C(=O)O] |
| phe(4-F) | 4- fluorophenylalanine | 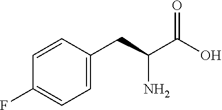 [SMILES: N[C@@H](Cc1ccc(F)cc1)C(=O)O] |
| Cha | cyclohexylalanine | 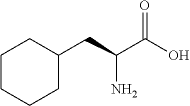 [SMILES: N[C@@H](CC1CCCCC1)C(=O)O] |
| h-phe | homophenylalanine | 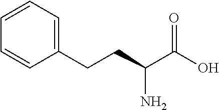 [SMILES: N[C@@H](CCc1ccccc1)C(=O)O] |
| phe(4-I) | 4-iodophenylalanine | 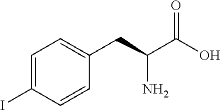 [SMILES: N[C@@H](Cc1ccc(I)cc1)C(=O)O] |
| phe(4-Cl) | 4- chlorophenylalanine | 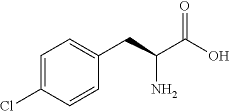 [SMILES: N[C@@H](Cc1ccc(Cl)cc1)C(=O)O] |
| D-phe | D-phenylalanine | 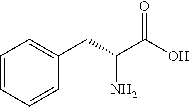 [SMILES: N[C@H](Cc1ccccc1)C(=O)O] |
| NaI | 3-(1-Naphthyl)- alanine | 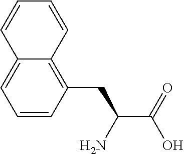 [SMILES: N[C@@H](Cc1cccc2ccccc12)C(=O)O] |
| phe(4-Br) | 4- bromophenylalanine | 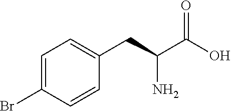 [SMILES: N[C@@H](Cc1ccc(Br)cc1)C(=O)O] |
| phe(4-Ph) (or F(Ph) or Phe(Ph)) | p-phenyl- phenylalanine | 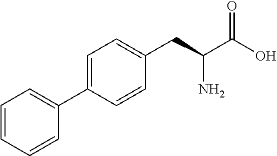 [SMILES: N[C@@H](Cc1ccc(-c2ccccc2)cc1)C(=O)O] |
| phe(4-CF₃) or phe(4CF₃) or phe(CF₃) | 4- trifluoromethylphenyl alanine | 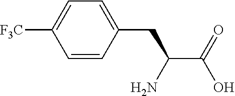 [SMILES: N[C@@H](Cc1ccc(C(F)(F)F)cc1)C(=O)O] |
| Cou | Courmarin | 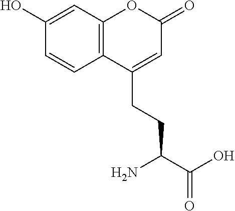 [SMILES: N[C@@H](CCc1cc(=O)oc2cc(O)ccc12)C(=O)O] |
| D-tyr | D-tyrosine | 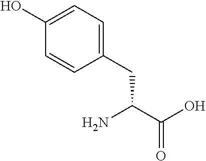 [SMILES: N[C@H](Cc1ccc(O)cc1)C(=O)O] |
| aib | 2-aminoisobutyric acid | 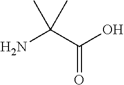 |
| n-leu | norleucine | 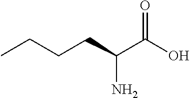 |
| Ser(OMe) Or Prop | O-methylserine | 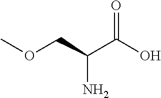 |
| Ile | Isoleucine | 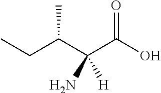 |
| Lys | Lysine | 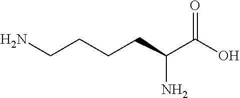 |
| Glu | Glutamic acid | 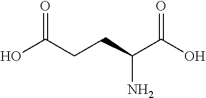 |
| h-cys | homocysteine | 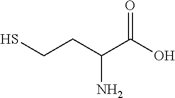 |
| d-cys | D-cysteine | 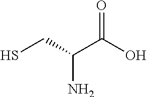 |
| abu | 2-aminobutanoic acid | 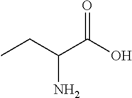 |
| h-leu | homoleucine | 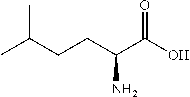 |
| n-val or nva or NV | norvaline | 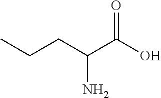 |
| h-cou | L-(7- hydroxycoumarin-4- yl) ethylglycine | 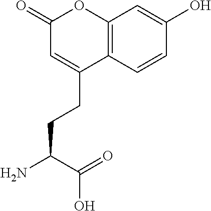 [SMILES: N[C@@H](CCc1cc(=O)oc2cc(O)ccc12)C(=O)O] |
| Phe(4-tBu) | 4-tert-butyl- phenylalanine | 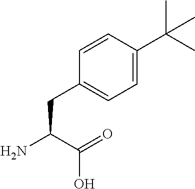 [SMILES: CC(C)(C)c1ccc(C[C@H](N)C(=O)O)cc1] |

### Preferred Groups of the Invention

The invention provides a cyclic peptide, wherein the cyclic peptide has the following sequence:

[Formula 1]
[SEQ ID NO: 1]
C X₁ X₂ X₃ X₄ X₅

wherein:
   i) C is selected from the group comprising or consisting of cys, h-cys and d-cys, optionally is cys;
   ii) X₁ is selected from the group comprising or consisting of leu, lys and glu, optionally is lys or leu, optionally is lys;
   iii) X₂ is:

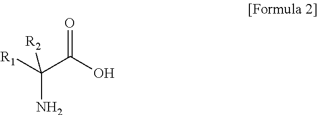

wherein:
   R₁ is H or methyl;
   R₂ is C₁-C₆ linear or branched alkyl, optionally substituted with —OC₁₋₃ alkyl;
iv) X₃ is:

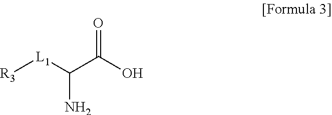

wherein:
   L₁ is C₁-C₃ alkylene;
   R₃ is selected from the group consisting of a C₃-C₆ cycloalkyl, a 5-6 membered ring and 8-10 membered bicyclic ring, wherein the rings are optionally substituted with phenyl, halogen, —OH, —O(C₁-C₃ alkyl), C₁-C₄ alkyl or C₁-C₄ haloalkyl;

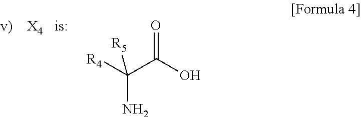

wherein:
   R₄ is H or methyl;
   R₅ is C₁-C₆ linear or branched alkyl, optionally substituted with —OC₁₋₃ alkyl;
vi) X₅ is:

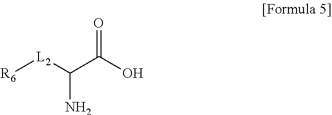

wherein:
   L₂ is C₁-C₃ alkylene or a direct bond;
   R₆ is selected from the group consisting of a C₃-C₆ cycloalkyl, 5-6 membered ring and 8-10 membered bicyclic ring, wherein the rings are optionally substituted with phenyl, halogen, —NO₂, —OH, —O(C₁-C₃ alkyl), C₁-C₄ alkyl or C₁-C₄ haloalkyl;
wherein:

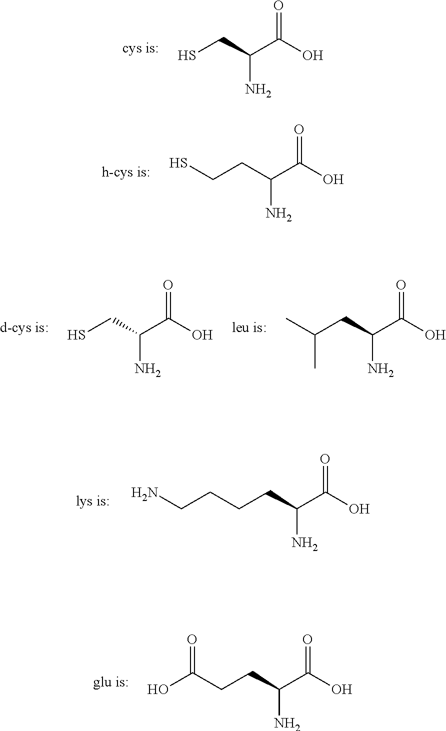

The skilled person will appreciate that C₁-C₃ alkylene includes structures such as a bivalent saturated aliphatic radical, for example methylene and ethylene.

The skilled person will also appreciate that a 5-6 membered ring includes within its meaning, for example, phenyl groups and heterocycle groups, for example pyridine groups; and that a 8-10 membered bicyclic ring includes within its meaning naphthalene (e.g. as in Nal) and coumarin (as in cou).

A “ring” as described herein, e.g. in the context of a 5-6 membered ring and an 8-10 membered ring, includes within its meaning aromatic and heteroaromatic rings. Bicyclic rings include within its meaning aromatic rings fused with non-aromatic heterocycle (e.g. benzopyrones, coumarin).

In some embodiments the cyclic peptide comprises at least one of:
X₂ wherein:
   (i) R₁ and R₂ are methyl (aib); or
   (ii) R₁ is H and R₂ is selected from the group consisting of (S)-isopentyl (h-leu), (S)-n-propyl (n-val), (S)-methoxymethyl (ser(OMe)), and (S)-n-butyl (n-leu), preferably
   (iii) R₁ is H and R₂ is selected from the group consisting of (S)-isopentyl (h-leu), (S)-n-propyl (n-val), (S)-methoxymethyl (ser(OMe)), and (S)-n-butyl (n-leu), more preferably;
   (iv) R₁ is H and R₂ is (S)-n-butyl (n-leu); or
X₃ wherein:
   (i) L₁ is (S)-methylene and R₃ is selected from the group consisting of cyclohexyl (Cha), 4-iodophenyl (phe(4-I)), 4-chlorophenyl (phe(4-Cl)), 4-bromophenyl (phe(4-Br)), 4-phenylphenyl (phe(4-Ph)), 4-trifluoromethylphenyl (phe(4-CF3)) and 1-naphtyl (NaI), or
   (ii) L₁ is (R)-methylene and R₃ is phenyl (d-Phe), or
   (iii) L₁ is ethylene, R₃ is selected from the group consisting of phenyl (h-Phe) and 7-hydroxy-4-coumarinyl (Cou), preferably
   (iv) L₁ is (S)-methylene and R₃ is selected from the group consisting of 4-iodophenyl (phe(4-I)), 4-chlorophenyl (phe(4-Cl)), 4-bromophenyl (phe(4-Br)), 4-phenylphenyl (phe(4-Ph)), and 4-trifluoromethylphenyl (phe(4-CF3)), 1-naphtyl (NaI), or
   (v) L₁ is (R)-methylene and R₃ is phenyl (d-Phe), or
   (vi) L₁ is ethylene and R₃ is 7-hydroxy-4-coumarinyl (Cou), more preferably
   (vii) L₁ is (S)-methylene and R₃ is 4-trifluoromethylphenyl (phe(4-CF3)); or
X₄ wherein:
   (i) R₄ and R₅ are methyl (aib),
   (ii) R₄ is H and R₅ is selected from the group consisting of ethyl (abu), (S)-n-butyl (n-leu) and (S)-methoxymethyl (ser(OMe)), preferably
   (iii) R₄ is H and R₅ is selected from (S)-n-butyl (n-leu) and (S)-methoxymethyl (ser(OMe)); or
X₅ wherein:
   (i) L₂ is (S)-methylene and R₆ is selected from the group consisting of 4-pyrid-tyrl (4-Pal), cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
   (ii) L₂ is (R)-methylene and Re is 4-hydroxyphenyl (d-tyr), or
   (iii) L₂ is a direct bond and Re is phenyl (phg), or
   (iv) L₂ is ethylene and Re is phenyl (h-phe), preferably
   (v) L₂ is (S)-methylene and Re is selected from the group consisting of cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
   (vi) L₂ is (R)-methylene and R₆ is 4-hydroxyphenyl (d-tyr), or
   (vii) L₂ is a direct bond and R₆ is phenyl (phg), or
   (viii) L₂ is ethylene and R₆ is phenyl (h-phe), more preferably
   (ix) L₂ is (S)-methylene and Re is selected from the group consisting of 4-iodophenyl (phe(4-I)), 4-fluorophenyl (phe(4-F)), 4-chlorophenyl (phe(4-Cl)), 4-bromophenyl (phe(4-Br)) and 4-nitrophenyl (phe(4-NO₂)), or
   (x) L₂ is ethylene and Re is phenyl (h-phe), most preferably
   (xi) L₂ is (S)-methylene and Re is 4-bromophenyl (phe(4-Br)).

In some embodiments the cyclic peptide of the invention R₄ is H and R₅ is (S,S)-2-butyl (ile).

In some embodiments the cyclic peptide of the invention, where R₄ is H and R₅ is (S,S)-2-butyl (ile), then
(A)
(i) R₁ and R₂ are methyl (aib), or
(ii) R₁ is H and R₂ is selected from the group consisting of (S)-isopentyl (h-leu), (S)-n-propyl (n-val), (S)-methoxymethyl (ser(OMe)) and (S)-n-butyl (n-leu), preferably
(iii) R₁ is H and R₂ is selected from the group consisting of (S)-isopentyl (h-leu), (S)-n-propyl (n-val), (S)-methoxymethyl (ser(OMe)) and (S)-n-butyl (n-leu), more preferably
(iv) R₁ is H and R₂ is (S)-n-butyl (n-leu); and
(B)
(i) L₁ is (S)-methylene and R₃ is selected from the group consisting of cyclohexyl (Cha), 4-iodophenyl (phe(4-I)), 4-chlorophenyl (phe(4-Cl)), 4-bromophenyl (phe(4-Br)), 4-phenylphenyl (phe(4-Ph)), 4-trifluoromethylphenyl (phe(4-CF3)) and 1-naphtyl (NaI), or
(ii) L₁ is (R)-methylene and R₃ is phenyl (d-Phe), or
(iii) L₁ is ethylene, R₃ is selected from the group consisting of phenyl (h-Phe) and 7-hydroxy-4-coumarinyl (Cou), preferably
(iv) L₁ is (S)-methylene and R₃ is selected from the group consisting of 4-iodophenyl (phe(4-I)), 4-chlorophenyl (phe(4-Cl)), 4-bromophenyl (phe(4-Br)), 4-phenylphenyl (phe(4-Ph)), and 4-trifluoromethylphenyl (phe(4-CF3)), 1-naphtyl (NaI), or
(v) L₁ is (R)-methylene and R₃ is phenyl (d-Phe), or
(vi) L₁ is ethylene and R₃ is 7-hydroxy-4-coumarinyl (Cou), more preferably
(vii) L₁ is (S)-methylene and R₃ is 4-trifluoromethylphenyl (phe(4-CF3)); and
(C)
(i) L₂ is (S)-methylene and Re is selected from the group consisting of 4-pyridyl (4-Pal), cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
(ii) L₂ is (R)-methylene and R₆ is 4-hydroxyphenyl (d-tyr), or
(iii) L₂ is a direct bond and R₆ is phenyl (phg), or
(iv) L₂ is ethylene and R₆ is phenyl (h-phe), preferably
(v) L₂ is (S)-methylene and Re is selected from the group consisting of cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
(vi) L₂ is (R)-methylene and R₆ is 4-hydroxyphenyl (d-tyr), or
(vii) L₂ is a direct bond and R₆ is phenyl (phg), or
(viii) L₂ is ethylene and Re s phenyl (h-phe), more preferably
(ix) L₂ is (S)-methylene and R₆ is selected from the group consisting of 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), and 1-naphtyl (NaI), or
(x) L₂ is ethylene and Re is phenyl (h-phe), most preferably
(xi) L₂ is (S)-methylene and R₆ is 4-bromophenyl (phe(4-Br)).

In some embodiments, the cyclic peptides of the invention have a sequence that conforms to the following consensus sequence:

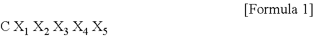

wherein:
   i) C is selected from the group comprising or consisting of cys, h-cys and d-cys. In preferred embodiments C is cys;
   ii) X₁ is selected from the group comprising or consisting of leu, lys and glu. In preferred embodiments X₁ lys or leu. In more preferred embodiments X₁ is lys;
   iii) X₂ is selected from the group comprising or consisting leu, val, aib, h-leu, n-val, ser(OMe), lie, n-leu. In preferred embodiments X₂ is selected from the group comprising or consisting of h-leu, n-val, ser(OMe), lie, n-leu. In further preferred embodiments X₂ is selected from the group comprising or consisting of ser(OMe) or ile;
   iv) X₃ is selected from the group comprising or consisting of phe, Cha and h-phe, phe(4-I), phe(4-Cl), d-phe, NaI, phe(4-Br), phe(4-Ph), phe(4-CF₃), Cou, and phe(4-tBu). In preferred embodiments X₃ s selected from the group comprising or consisting of phe(4-I), phe(4-Cl), d-phe, NaI, phe(4-Br), phe(4-Ph), phe(4-CF₃), Cou and phe(4-tBu). In preferred embodiments X₃ is phe(4-CF₃);
   v) X₄ is selected from the group comprising or consisting val, aib, abu, n-leu, ser(OMe), leu, lie. In preferred embodiments X₄ is selected from the group comprising or consisting of n-leu, ser(OMe), leu, ile. In more preferred embodiments X₄ is selected from the group comprising or consisting of leu, ile. In more preferred embodiments X₄ is ile; and
   vi) X₅ is selected from the group comprising or consisting tyr, 4-Pal, Cha, phe(4-Ph), d-tyr, phg, phe(4-CF₃), phe, tyr(Me), phe(4-I), h-phe, phe(4-NO₂), Nal, phe(4-F), phe(4-Cl), phe(4-Br) and phe(4-tBu). In preferred embodiments X₅ is selected from the group comprising or consisting of Cha, phe(4-Ph), d-tyr, phg, phe(4-CF₃), phe, tyr(Me), phe(4-I), h-phe, phe(4-NO₂), Nal, phe(4-F), phe(4-Cl), phe(4-Br) and phe(4-tBu). In more preferred embodiments X₅ is selected from the group comprising or consisting of phe(4-I), h-phe, phe(4-NO₂), Nal, phe(4-F), phe(4-Cl), phe(4-Br) and phe(4-tBu). In more preferred embodiments X₅ is phe(4-Br);
wherein:

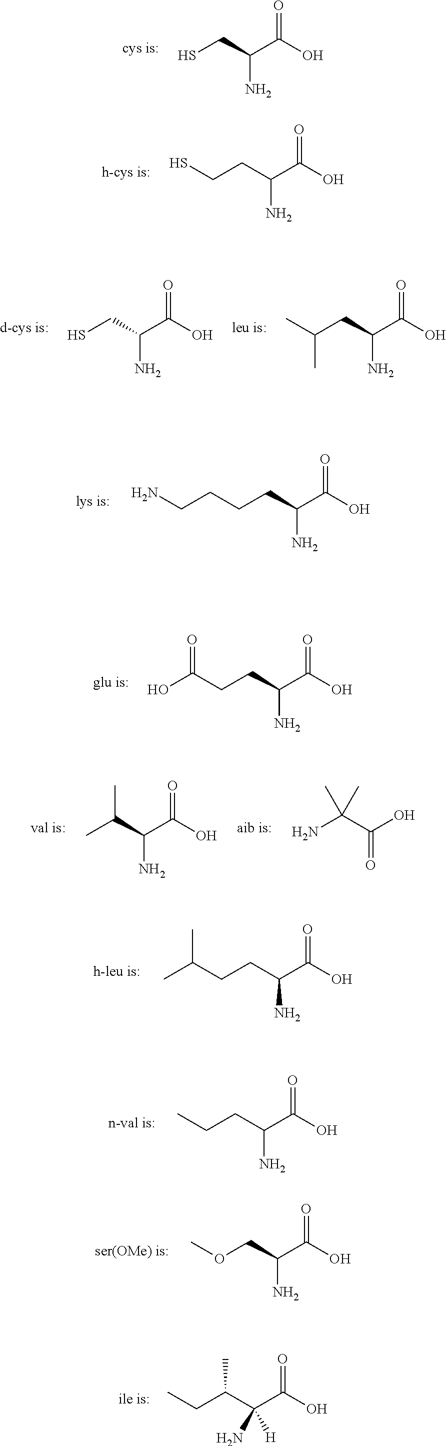

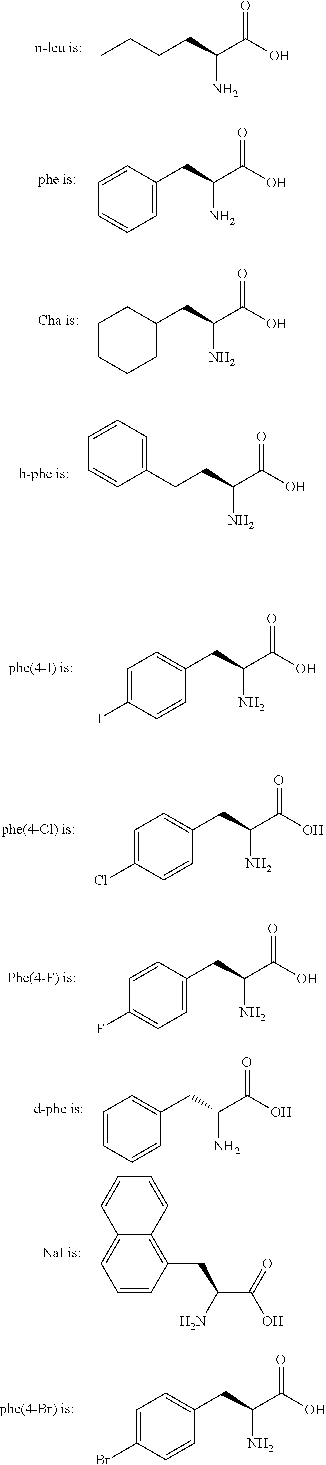

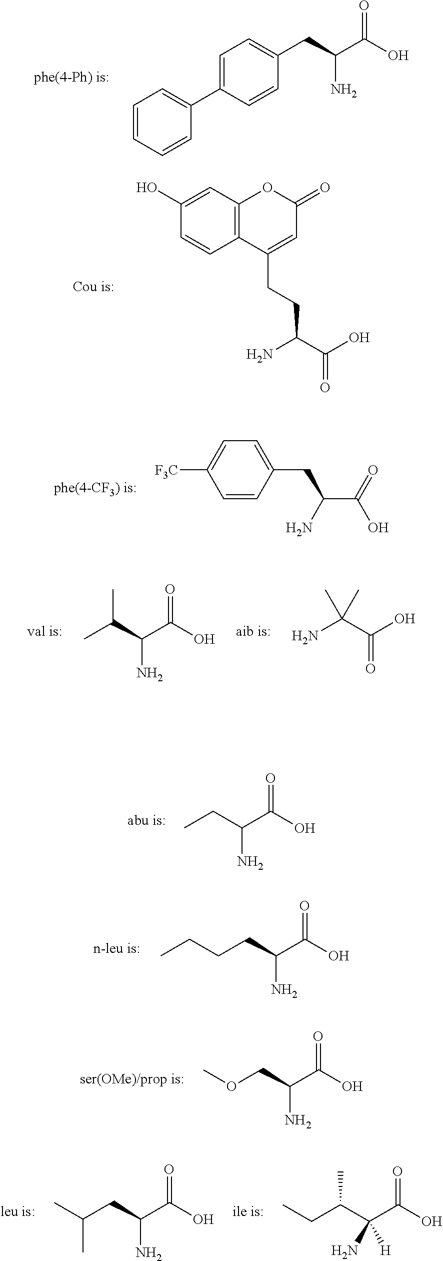

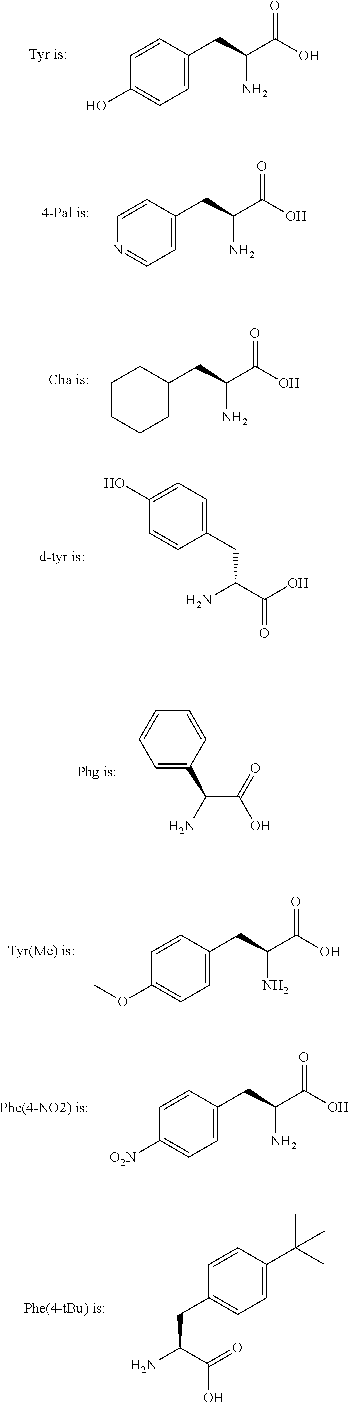

In some embodiments, the cyclic peptide comprises at least one of:
X₂ selected from aib, h-leu, n-val, ser(OMe), n-leu, optionally selected from h-leu, n-val, ser(OMe), and n-leu, optionally n-leu; or
X₃ selected from Cha, h-phe, phe(4-I), phe(4-Cl), d-phe, NaI, phe(4-Br), phe(4-Ph), phe(4-CF₃), Cou and phe(4-tBu), optionally selected from phe(4-I), phe(4-Cl), d-phe, NaI, phe(4-Br), phe(4-Ph), phe(4-CF₃), phe(4-tBu) and Cou, optionally is phe(4-CF₃); or
X₄ is selected from aib, abu, n-leu or ser(OMe), optionally selected from n-leu or ser(Ome); or
X₅ is selected from 4-Pal, Cha, phe(4-Ph), d-tyr, phg, phe(4-CF₃), tyr(Me), phe(4-I), h-phe, phe(4-NO₂), Nal, phe(4-F), phe(4-Cl), phe(4-tBu) or phe(4-Br), optionally selected from Cha, phe(4-Ph), d-tyr, phg, phe(4-CF₃), tyr(Me), phe(4-I), h-phe, phe(4-NO)), Nal, phe(4-F), phe(4-Cl), phe(4-tBu) and phe(4-Br), optionally selected from phe(4-I), h-phe, phe(4-NO₂), Nal, phe(4-F), phe(4-Cl), phe(4-tBu), phe(4-Br), optionally is phe(4-Br).

In some further embodiments, where X₄ is ile, then:
X₂ is selected from aib, h-leu, n-val, ser(OMe), n-leu, optionally selected from h-leu, n-val, ser(OMe), and n-leu, optionally n-leu; or
X₃ is selected from Cha, h-phe, phe(4-I), phe(4-Cl), d-phe, NaI, phe(4-Br), phe(4-Ph), phe(4-CF₃) and Cou, optionally selected from phe(4-I), phe(4-Cl), d-phe, NaI, phe(4-Br), phe(4-Ph), phe(4-CF₃) and Cou, optionally is phe(4-CF₃); or
X₅ is selected from 4-Pal, Cha, phe(4-Ph), d-tyr, phg, phe(4-CF₃), tyr(Me), phe(4-I), h-phe, phe(4-NO₂), Nal, phe(4-F), phe(4-Cl) or phe(4-Br), optionally selected from Cha, phe(4-Ph), d-tyr, phg, phe(4-CF₃), tyr(Me), phe(4-I), h-phe, phe(4-NO₂), Nal, phe(4-F), phe(4-Cl) and phe(4-Br), optionally selected from phe(4-I), h-phe, phe(4-NO₂), Nal, phe(4-F), phe(4-Cl), phe(4-Br), optionally is phe(4-Br).

In some other embodiments, where X₄ is Ile, then
X₂ selected from Aib, h-leu, n-val, Prop, or n-leu, optionally selected from h-leu, n-val, Prop or n-leu, optionally n-leu; and
X₃ selected from ha, h-phe, phe(4-I), phe(4-Cl), d-phe, NaI, phe(4-Br), phe(4-Ph), phe(CF₃) and Cou; optionally phe(I), phe(Cl), d-phe, NaI, phe(4-Br), phe(4-Ph), phe(CF₃) and Cou; optionally phe(CF₃); and
X₄ is selected from Abu, Aib, n-leu, or Prop; optionally n-leu or Prop; and
X₅ is selected from Pal, Cha, phe(4-Ph), d-tyr, Phg, phe(CF₃), Tyr(me), phe(4-I), h-phe, phe(NO2), NaI, phe(4-F), phe(4-Cl), or phe(4-Br); optionally Cha, phe(4-Ph), d-tyr, Phg, phe(CF₃), Tyr(me), phe(4-I), h-phe, phe(NO₂), NaI, phe(4-F), phe(4-Cl), or phe(4-Br); optionally phe(4-I), h-phe, phe(NO₂), NaI, phe(4-F), phe(4-Cl), or phe(4-Br); optionally phe(4-Br).

In some or all embodiments, the cyclic peptide of the invention does not comprise the sequence:
Cys leu leu phe val tyr
[SEQ ID NO: 44]

It will be clear to the skilled person that the invention provides cyclic peptides with particular sequences.

For example, in one embodiment the cyclic peptide of the invention comprises, or consists of, one of the following sequences:

[SEQ ID NO: 2]
Cys leu leu Cha val tyr
[SEQ ID NO: 3]
Cys leu leu h-phe val tyr
[SEQ ID NO: 4]
Cys leu leu phe(4-I) val tyr
[SEQ ID NO: 5]
Cys leu leu phe(4-Cl) val tyr
[SEQ ID NO: 6]
Cys leu leu d-phe val tyr
[SEQ ID NO: 7]
Cys leu leu Nal val tyr
[SEQ ID NO: 8]
Cys leu leu phe(4-Br) val tyr
[SEQ ID NO: 9]
Cys leu leu phe(4-Ph) val tyr
[SEQ ID NO: 10]
Cys leu leu phe(CF₃) val tyr
[SEQ ID NO: 39]
Cys leu leu cou val tyr
[SEQ ID NO: 56]
Cys leu leu phe(4-tBu) val tyr

In preferred embodiments the cyclic peptide of the invention comprises or consists of one of the following sequences:

[SEQ ID NO: 4]
Cys leu leu phe(4-I) val tyr
[SEQ ID NO: 5]
Cys leu leu phe(4-Cl) val tyr
[SEQ ID NO: 6]
Cys leu leu d-phe val tyr
[SEQ ID NO: 7]
Cys leu leu Nal val tyr
[SEQ ID NO: 8]
Cys leu leu phe(4-Br) val tyr
[SEQ ID NO: 9]
Cys leu leu phe(4-Ph) val tyr
[SEQ ID NO: 10]
Cys leu leu phe(CF₃) val tyr
[SEQ ID NO: 39]
Cys leu leu cou val tyr
[SEQ ID NO: 56]
Cys leu leu phe(4-tBu) val tyr

In more preferred embodiments the cyclic peptide of the invention comprises or consists of the following sequence:

[SEQ ID NO: 10]
Cys leu leu phe(CF₃) val tyr
[SEQ ID NO: 56]
Cys leu leu phe(4-tBu) val tyr

In more preferred embodiments the cyclic peptide of the invention comprises or consists of the following sequence:

[SEQ ID NO: 10]
Cys leu leu phe(CF₃) val tyr

The invention also provides a cyclic peptide that comprises, or consists of, one of the following sequences:

[SEQ ID NO: 11]
Cys leu leu phe(CF₃) val PaI
[SEQ ID NO: 12]
Cys leu leu phe(CF₃) val Cha
[SEQ ID NO: 13]
Cys leu leu phe(CF₃) val phe(4-Ph)
[SEQ ID NO: 14]
Cys leu leu phe(CF₃) val d-tyr
[SEQ ID NO: 15]
Cys leu leu phe(CF₃) val Phg
[SEQ ID NO: 16]
Cys leu leu phe(CF3) val phe(4-CF3)
[SEQ ID NO: 17]
Cys leu leu phe(CF₃) val phe
[SEQ ID NO: 18]
Cys leu leu phe(CF₃) val Tyr(me)
[SEQ ID NO: 19]
Cys leu leu phe(CF₃) val phe(4-I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(4-F)
[SEQ ID NO: 24]
Cys leu leu phe(CF₃) val phe(4-Cl)
[SEQ ID NO: 25]
Cys leu leu phe(CF₃) val phe(4-Br)
[SEQ ID NO: 57]
Cys leu leu phe(CF₃) val phe(4-tBu)

In preferred embodiments the cyclic peptide of the invention comprises or consists of one of the following sequences:

[SEQ ID NO: 12]
Cys leu leu phe(CF₃) val Cha
[SEQ ID NO: 13]
Cys leu leu phe(CF₃) val phe(4-Ph)
[SEQ ID NO: 14]
Cys leu leu phe(CF₃) val d-tyr
[SEQ ID NO: 15]
Cys leu leu phe(CF₃) val Phg
[SEQ ID NO: 16]
Cys leu leu phe(CF3) val phe(4-CF3)
[SEQ ID NO: 17]
Cys leu leu phe(CF₃) val phe
[SEQ ID NO: 18]
Cys leu leu phe(CF₃) val Tyr(me)
[SEQ ID NO: 19]
Cys leu leu phe(CF₃) val phe(4-I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(4-F)
[SEQ ID NO: 24]
Cys leu leu phe(CF₃) val phe(4-Cl)
[SEQ ID NO: 25]
Cys leu leu phe(CF₃) val phe(4-Br)
[SEQ ID NO: 57]
Cys leu leu phe(CF₃) val phe(4-tBu)

In more preferred embodiments the cyclic peptide of the invention comprises or consists of one of the following sequences:

[SEQ ID NO: 19]
Cys leu leu phe(CF₃) val phe(4-I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(4-F)
[SEQ ID NO: 24]
Cys leu leu phe(CF₃) val phe(4-Cl)
[SEQ ID NO: 25]
Cys leu leu phe(CF₃) val phe(4-Br)
[SEQ ID NO: 57]
Cys leu leu phe(CF₃) val phe(4-tBu)

In more preferred embodiments the cyclic peptide of the invention comprises or consists of the following sequence:

[SEQ ID NO: 25[
Cys leu leu phe(CF₃) val phe(4-Br).

The invention also provides cyclic peptides that comprise, or consist of, one of the following sequences:

[SEQ ID NO: 26]
Cys leu leu phe(CF₃) Abu phe(4-Br)
[SEQ ID NO: 27]
Cys leu leu phe(CF₃) Aib phe(4-Br)
[SEQ ID NO: 28]
Cys leu leu phe(CF₃) n-leu phe(4-Br)
[SEQ ID NO: 29]
Cys leu leu phe(CF₃) Prop phe(4-Br)
[SEQ ID NO: 30]
Cys leu leu phe(CF₃) leu phe(4-Br)
[SEQ ID NO: 31]
Cys leu leu phe(CF₃) ile phe(4-Br)
[SEQ ID NO: 58]
Cys leu leu phe(CF₃) n-val phe(4-Br)
[SEQ ID NO: 59]
Cys leu leu phe(CF₃) h-leu phe(4-Br)

In preferred embodiments the cyclic peptide of the invention comprises or consists of one of the following sequences:

[SEQ ID NO: 28]
Cys leu leu phe(CF₃) n-leu phe(4-Br)
[SEQ ID NO: 29]
Cys leu leu phe(CF₃) Prop phe(4-Br)
[SEQ ID NO: 30]
Cys leu leu phe(CF₃) leu phe(4-Br)
[SEQ ID NO: 31]
Cys leu leu phe(CF₃) ile phe(4-Br)
[SEQ ID NO: 59]
Cys leu leu phe(CF₃) h-leu phe(4-Br)

In more preferred embodiments the cyclic peptide of the invention comprises or consists of one of the following sequences:

[SEQ ID NO: 30]
Cys leu leu phe(CF₃) leu phe(4-Br)
[SEQ ID NO: 31]
Cys leu leu phe(CF₃) ile phe(4-Br)
[SEQ ID NO: 59]
Cys leu leu phe(CF₃) h-leu phe(4-Br)

In some embodiments the cyclic peptide comprises or consists of the following sequence:

[SEQ ID NO: 31]
Cys leu leu phe(CF₃) ile phe(4-Br).

In some embodiments the cyclic peptide of the invention comprises or consists of one of the following sequences:

[SEQ ID NO: 32]
Cys leu val phe(CF₃) ile phe(4-Br)
[SEQ ID NO: 33]
Cys leu Aib phe(CF₃) ile phe(4-Br)
[SEQ ID NO: 34]
Cys leu h-leu phe(CF₃) ile phe(4-Br)
[SEQ ID NO: 35]
Cys leu n-val phe(CF₃) ile phe(4-Br)
[SEQ ID NO: 36]
Cys leu Prop phe(CF₃) ile phe(4-Br)
[SEQ ID NO: 37]
Cys leu ile phe(CF₃) ile phe(4-Br)
[SEQ ID NO: 38]
Cys leu n-leu phe(CF₃) ile phe(4-Br),

In preferred embodiments the cyclic peptide of the invention comprises or consists of one of the following sequences:

[SEQ ID NO: 34]
Cys leu h-leu phe(CF₃) ile phe(4-Br)
[SEQ ID NO: 35]
Cys leu n-val phe(CF₃) ile phe(4-Br)
[SEQ ID NO: 36]
Cys leu Prop phe(CF₃) ile phe(4-Br)
[SEQ ID NO: 37]
Cys leu ile phe(CF₃) ile phe(4-Br)
[SEQ ID NO: 38]
Cys leu n-leu phe(CF₃) ile phe(4-Br).

In some embodiments the cyclic peptide of the invention comprises or consists of the following sequence:

[SEQ ID NO: 38]
Cys leu n-leu phe(CF₃) ile phe(4-Br).

In some embodiments the cyclic peptide of the invention comprises or consists of one of the following sequences:

[SEQ ID NO: 40]
Cys lys n-leu phe(CF₃) ile phe(4-Br)
[SEQ ID NO: 41]
Cys glu n-leu phe(CF₃) ile phe(4-Br)

In some embodiments the cyclic peptide of the invention comprises or consists of one of the following sequences:

[SEQ ID NO: 42]
h-cys leu leu phe val tyr
[SEQ ID NO: 43]
d-cys leu leu phe val tyr

It will be clear to the skilled person that any of the residues in the cyclic peptide can be a D or an L amino acid. Accordingly, in one embodiment any one or more of cys, X₁, leu, X₂, X₃, or X₄ is a D amino acid or derivative thereof. In another embodiment, any one or more of cys, X₁, leu, X₂, X₃, X₄ is an L amino acid or derivative thereof. In another embodiment, the cyclic peptide comprises both L and D amino acids or derivatives thereof.

As described above, the cyclic peptides of the present invention are considered to be useful in inhibiting the interaction between HIF-α and HIF1β.

Accordingly, in one embodiment the cyclic peptide is an inhibitor of the interaction between HIF-2α and HIF-1β. In one embodiment the cyclic peptide is capable of binding to HIF-1α, for example is capable of binding to recombinantly expressed PAS-B domain of HIF-1α.

In another or the same embodiment, the cyclic peptide is an inhibitor of the interaction between HIF-1α and HIF-1β.

The skilled person will understand what is meant by “capable of binding to HIF-1α”.

The cyclic peptide may bind to any region of HIF-1α.

The skilled person will recognise that there are multiple means to determine the binding affinity of the cyclic peptide to HIF-1α or to recombinantly expressed PAS-B domain of HIF-1α. For example, in one embodiment the affinity of determined using microscale thermophoresis, for example is determining against the recombinantly expressed PAS-B domain of HIF-1α using microscale thermophoresis.

The skilled person will understand when the affinity to which the peptide binds to HIF-1α is sufficient so as to render the peptide particular useful. For example, in one embodiment the peptide binds to HIF-1α with a Kd of:
less than 40 μM, 37 μM, 36 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2.5 μM, 2 μM, 1.5 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 40 μM, 0.2 μM and 37 μM, 0.3 μM and 36 μM, 0.4 μM and 35 μM, 0.5 μM and 30 μM, 0.6 UM and 25 UN, 0.7 μM and 20 μM, 0.8 μM and 18 μM, 0.9 μM and 16 μM, 1 μM and 15 μM, 2 μM and 14 μM, 3 μM and 13 μM, 4 μM and 12 μM, 5 μM and 11 μM, 6 μM and 10 μM, 7 μM and 9 μM
   for example binds to binds to HIF-1α with a Kd of any of the above wherein the affinity is determined against HIF-1α using microscale thermophoresis.

As described above, the ability of a cyclic peptide to bind to HIF-1α can be determined by determining the ability of the cyclic peptide to bind to recombinantly expressed PAS-B domain of HIF-1α. Accordingly, in one embodiment the peptide binds to recombinantly expressed PAS-B domain of HIF-1α with a Kd of:
less than 40 μM, 37 μM, 36 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2.5 μM, 2 μM, 1.5 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 40 μM, 0.2 μM and 37 μM, 0.3 μM and 36 μM, 0.4 μM and 35 μM, 0.5 μM and 30 μM, 0.6 μM and 25 μM, 0.7 μM and 20 μM, 0.8 μM and 18 μM, 0.9 μM and 16 μM, 1 μM and 15 μM, 2 μM and 14 μM, 3 μM and 13 μM, 4 μM and 12 μM, 5 μM and 11 μM, 6 μM and 10 μM, 7 μM and 9 μM for example binds to binds to HIF-1α with a Kd of any of the above wherein the affinity is determined against the recombinantly expressed PAS-B domain of HIF-1α using microscale thermophoresis.

The cyclic peptides are considered to be particularly useful if they are able to disrupt the interaction between the α and β subunits of the HIF heterodimeric protein, i.e. prevent association or binding of the α and β subunits of the HIF heterodimeric protein. In one embodiment the cyclic peptides of the invention are able to disrupt the interaction between HIF-1α and HIP-1β. In the same or different embodiment, the cyclic peptides of the invention are able to disrupt the interaction between HIF-2α and HIF-1β. In a preferred embodiment, the cyclic peptides are able to disrupt the interaction between HIP-1α and HIF-1β, and between HIF-2α and HIF-1β.

The skilled person will understand how to determine whether a particular cyclic peptide is able to disrupt the interaction between two particular subunits. For example, various methods are used to determine protein-protein interactions, including yeast two hybrid assays and protein cross linking methods. The proximity ligation assay may also be used, whereby the interacting partner domains are targeted by separate primary and secondary antibodies. The secondary antibodies comprise PLA probes that contain short DNA strands. When in close proximity, i.e. where the interacting partner domains are contacting one another, the DNA strands can be amplified via rolling circle DNA synthesis. Identification of an amplification product indicates an interaction between the two partner domains. The proximity ligation assay can be performed in situ, for example in cells, for example in MCF-7 cells.

Accordingly, in one embodiment the cyclic peptide disrupts the interaction between HIF-1α and HIF-1β; between HIF-2α and HIF-1β; or between HIF-1α and HIP-1β, and between HIF-2α and HIF-1β, wherein the interaction is assessed by proximity ligation assay, optionally in MCF-7 cells.

As described above, the cyclic peptides of the invention are able to disrupt the interaction between HIF-1α and HIF-1β and so disrupt hypoxia induced expression of the hypoxia response by stopping the induction of expression from promoters that comprise one or more hypoxia-responsive elements. Accordingly, in one embodiment the cyclic peptides of the invention are able to disrupt the interaction between HIF-1α and HIF-1β with an IC50 of:
less than 50 μM, optionally less than 45 μM, 40 μM, 35 μM, 30 μM, 25 μM, 20 μM, 15 μM, 10 μM or less than 5 μM.

In some embodiments the cyclic peptides of the invention are able to disrupt the interaction between recombinantly expressed PAS-B domains from HIF-1α-HIF-1β with an IC50 of:
less than 50 μM, optionally less than 45 μM, 40 μM, 35 μM, 30 μM, 25 μM, 20 μM, 15 μM, 10 μM or less than 5 μM.

It will be clear from the discussion herein that in some embodiments the cyclic peptide of the invention is able to disrupt the interaction between recombinantly expressed HIF-1 and a HRE element in a section of DNA with an IC50 of:
less than 75 μM, optionally less than 70 μM, 65 μM, 60 μM, 55 μM, 50 μM, 45 μM, 40 μM, 35 μM, 30 μM, 25 μM, 20 μM, 15 μM, 10 μM or less than 5 μM.

In some embodiments the cyclic peptide of the invention is able to disrupt the interaction between HIP-1 and a HRE element in a section of DNA with an IC50 of:
less than 75 μM, optionally less than 70 μM, 65 μM, 60 μM, 55 μM, 50 μM, 45 μM, 40 μM, 35 μM, 30 μM, 25 μM, 20 μM, 15 μM, 10 μM or less than 5 μM.

Since the cyclic peptides of the invention are able to interfere with binding between HIF-1α-HIF-1β and binding of HIF-1 with a HRE element, it will be apparent to the skilled person that the cyclic peptides of the invention are useful in the preventing or reducing the response to hypoxia. Accordingly, in one embodiment the cyclic peptide of the invention prevents or reduces the hypoxia induced expression from a promoter that comprises one or more hypoxia-responsive elements under hypoxic conditions. The skilled person will understand what is meant by “hypoxia induced expression”. Hypoxia is a state of reduced oxygen concentration that can arise under normal conditions such as embryonic development, and in, for example, the tumour microenvironment. Examples of hypoxic conditions include. The skilled person will also understand what is meant by a hypoxia-responsive element (HRE).

For example, as described in the examples, a peptide of the invention prevents or reduces hypoxia induced expression from a promoter that comprises one or more hypoxia-responsive elements when the peptide prevents or reduces expression of a reporter protein, for example a yellow fluorescent reporter protein (YFP) which is under the control of a promoter with three copies of the HRE sequence in a cell, for example in a HEK cell line, for example in the T-REx-293 cell line, where the cell is exposed to hypoxic conditions. In the absence of a cyclic peptide of the invention, hypoxic conditions would result in expression of the reporter (e.g. YFP) as it is under the control of a promoter that comprises HRE elements. In the presence of a cyclic peptide of the invention, this increase in expression is prevented. See for example FIG. 4.

It will be understood by the skilled person that the term “expression” includes the meaning of transcription of a non-coding gene into a non-coding RNA, for example into an miRNA or a siRNA; as well as including the meaning of transcription of a coding gene into a coding RNA/mRNA and subsequent translation into a peptide or polypeptide.

In one embodiment, the cyclic peptide reduces the hypoxia induced expression from a promoter that comprises one or more hypoxia-responsive elements under hypoxic conditions to less than 90%, 80%, 70%, 60%, 50%, 40%, 30%, 20%, 10%, 5%, 4%, 3%, 2%, 1% of the expression obtained in the absence of the cyclic peptide under hypoxic conditions. For example in one embodiment, the cyclic peptide reduces the hypoxia induced expression of a reporter protein, for example YFP, from a promoter that comprises one or more hypoxia-responsive elements, for example from a promoter that comprises three HRE sequences, under hypoxic conditions.

It is considered, in some embodiments, the solubility of the cyclic peptide is an important factor. In some embodiments the cyclic peptide of the invention has a solubility profile of:
less than 10 C Log P, optionally less than 9.5, 9.0, 8.5, 8.0, 7.5, 7.0, 6.5, 6.0, 5.5, 5.0 C Log P.

It will be apparent to the skilled person that as the invention provides cyclic peptides, the invention also provides corresponding polynucleotides that comprise or consists of a sequence that encodes the cyclic peptides of the invention. The invention provides a DNA polynucleotide that comprises or consists of a sequence that encodes the cyclic peptide of the invention. The invention also provides an RNA polynucleotide that comprises or consists of a sequence that encodes the cyclic peptide of the invention. The skilled person will appreciate that a polynucleotide, for example a DNA or RNA polynucleotide, may comprise one or more modifications, for example a phosphorothioate modification. The polynucleotide may also comprise one or more other features, for example a promoter, terminator, or a tag for instance, for example the features typical of an expression cassette.

The polynucleotide of the invention may also comprise one or more features that facilitate the cyclisation of the peptide. For example, the polynucleotide may comprise one or more sequences that allows Split-intein circular ligation of peptides and proteins (SICLOPPS) to be performed. For example, the polynucleotide may comprise portions of a split intein which facilitates circularisation of the peptide of the invention.

Accordingly the invention provides a polynucleotide comprising or consisting of a sequence that encodes an N-terminal intein fragment, followed by a sequence encoding a cyclic polypeptide according to any one of the preceding claims, followed by a sequence encoding a C-terminal intein fragment.

It will be clear to the skilled person that encoding the cyclic peptide of the invention in a nucleic acid is generally only suitable where the cyclic peptide comprises naturally occurring amino acids. However it will be appreciated that it is possible to artificially modify the genetic code in which one or more specific codons have been re-allocated to encode an amino acid that is not among the 22 common naturally-encoded proteinogenic amino acids.

The invention also provides a nucleic acid vector comprising the nucleic acid of the invention. The skilled person will understand that by nucleic acid vector we include the meaning of a plasmid, artificial chromosome or other nucleic acid structure used to deliver or express the cyclic peptide. The artificial chromosome may be any artificial chromosome and may be selected from, for example, a bacterial artificial chromosome (BAC), a yeast artificial chromosome (YAC), and a Human artificial chromosome (HAG). The invention also provides a cell that comprises the cyclic peptide of the invention, the polynucleotide of the invention and/or the vector of the invention. The cell of the invention has two main uses, amongst others—i) manufacture of the cyclic peptides or viral vectors comprising the cyclic peptides of the invention, for example; and ii) medical uses for example screening for suitable cyclic peptides for particular situations, or as a therapeutic cell.

In one embodiment then the cell is a cell that is used in the commercial, large scale manufacture of the cyclic peptides of the invention, for example is a bacterial cell such as E. coli, or is a yeast cell such as P. pastoris.

In another embodiment the cell is a cell that either is a direct “diseased” cell, for example taken from a biopsy from a patient. In another embodiment the cell is a cell that is intended to mimic or model a particular disease state. Such cells can be used to screen for appropriate cyclic peptides that are suitable for use in particular therapeutic situations.

Since the cyclic peptides of the invention are able to disrupt the typical response to hypoxia, it will be apparent to the skilled person that the cyclic peptide of the invention, the polynucleotide of the invention and/or the vector of the invention have use in the treatment and/or prevention of diseases, disorders or conditions. For example, in one embodiment the cyclic peptides of the invention are useful in the treatment or prevention of a disease, disorder or condition that experiences a hypoxic environment and requires the typical hypoxia response for maintenance. The cyclic peptides of the invention are also suitable for treatment or prevention of any other disease treatable or preventable by inhibition of dimerization of HIF-1a with HIF1-b and HIF2a with HIF1b and/or inhibits the activity of HIF-1 and HIF-2 and/or HIF-1 or HIF-2 signalling. The cyclic peptides of the invention are also suitable for use in the treatment or prevention of a disease, disorder or condition in which it is desirable to repress hypoxia induced gene expression.

Such diseases, disorders and conditions include Von Hippel-Lindau disease, tumours and cancer.

A tumour is not necessarily the same as cancer. By tumour we include the meaning of any kind of aberrant growth, whether it is benign or malignant. By cancer we include solid cancers and blood cancers. Solid cancers typically refer to an aberrant growth that is or has the potential to be malignant. Blood cancers are not solid cancers and include, for example, lymphomas.

Solid tumours and solid cancers in particular are known to experience a hypoxic tumour microenvironment, and it is known that a hypoxic tumour microenvironment correlates with poor prognosis. Blocking the response to hypoxia using the cyclic peptides of the invention is considered to be useful in the treatment and/or prevention of these diseases, disorders and conditions.

Accordingly, in one embodiment the cell is a human cell. In yet another embodiment the cell is a diseased cell, for example is a cancer cell. In one embodiment the cell is an in vitro cell, such as an in vitro mammalian cell or in vitro human cell. For example, in vitro human cells comprising the peptide, polynucleotide or vector of the invention may be used as part of a screening procedure to determine appropriate treatment strategies. In some embodiments the cell is not an in vivo human cell. In some embodiment the cell is not an in vivo human or animal cell.

In some instances, the isolated polynucleotide of the invention, or vector of the invention may be loaded into a viral vector, for example for therapeutic delivery. The invention therefore also provides a viral vector comprising the polynucleotide or vector of the invention. Viral vectors are well known in the art and examples include but are not limited to: adeno-associated viral vectors (AAV vectors); lentiviral vectors (e.g. those derived from Human Immunodeficiency Virus (HIV)); retroviral vectors (e.g. MMLV). In some embodiments the viral vector is selected from a group comprising a retroviral vector, a lentiviral vector, an adenoviral vector, an adeno-associated viral vector, a bacteriophage vector, and a hybrid viral vector.

In one embodiment the viral vector is not a viral vector that integrates into the genome of the host cell, for example such vectors include AAVs and adenoviral vectors. AAV vectors infect target cells and the delivered genetic material does not integrate into the genome of the host cell. Instead, the delivered genetic material remains episomal. In one embodiment the viral vector is a viral vector that integrates into the genome of the host cell, for example such vectors include the retroviral vectors, for example lentiviral vectors.

As described above, the cyclic peptides of the invention are useful in the treatment or prevention of disease, a disorder or condition. For example, in one embodiment the cyclic peptides of the invention are useful in the treatment or prevention of a disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIP-2 signalling; and/or
in which it is desirable to repress hypoxia induced gene expression.

Accordingly, the invention provides a pharmaceutical composition comprising one or more of the cyclic peptide of the invention, the polynucleotide of the invention, the vector of the invention or the viral vector of the invention.

As used herein, “pharmaceutical composition” means a therapeutically effective formulation.

For example in some embodiments the “pharmaceutical composition” is a therapeutically effective formulation for use in the treatment or prevention of diseases, disorders and conditions:
that experience a hypoxic environment and requires the typical hypoxia response for maintenance;
that are treatable or preventable by inhibition of dimerization of HIF-1a with HIF1-b; and/or
in which it is desirable to repress hypoxia induced gene expression.

Examples of such diseases, disorders and conditions includes cancer, such as solid cancer, or Von Hippel-Lindau disease.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the cyclic peptides, polynucleotides, vectors or viral vectors of the invention. Such pharmaceutically acceptable buffers, carriers, diluents or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcelluose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol or xylosides, and/or monoacyiglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

It will be appreciated that the pharmaceutical compositions may comprise one or more of the cyclic peptides, polynucleotides, vectors or viral vectors of the invention, for example one, two, three or four different the cyclic peptides, polynucleotides, vectors or viral vectors of the invention. By using a combination of different the cyclic peptides, polynucleotides, vectors or viral vectors of the invention the effect may be increased.

The pharmaceutical compositions of the invention may also be in the form of a liposome, in which the one or more cyclic peptides, polynucleotides, vectors or viral vectors of the invention is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, which is incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microshperes. Preparations of such microspheres can be found in U.S. Pat. No. 5,851,451 and in EP 213 303, which are incorporated herein by reference.

The pharmaceutical compositions of the invention may also be formulated with micellar systems formed by surfactants and block copolymers, preferably those containing poly(ethylene oxide) moieties for prolonging bloodstream circulation time.

The pharmaceutical compositions of the invention may also be in the form of polymer gels, where polymers such as starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propylcellulose, alginates, chitosan, carageenans, hyaluronic acid and derivatives thereof, polyacrylic acid, polyvinyl imidazole, polysulphonate, polyethylenglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

Alternatively, the cyclic peptides, polynucleotides, vectors or viral vectors of the invention may be dissolved in saline, water, polyethylene glycol, propylene glycol, ethanol or oils (such as safflower oil, corn oil, peanut oil, cottonseed oil or sesame oil), tragacanth gum, and/or various buffers.

The pharmaceutical composition may also include ions and a defined pH for potentiation of action of anti-microbial polypeptides.

The above compositions of the invention may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions of the invention may be administered locally or systemically. Routes of administration include topical (e.g. ophthalmic), ocular, nasal, pulmonary, buccal, parenteral (intravenous, subcutaneous, and intramuscular), oral, vaginal and rectal. Also administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used as described above. The pharmaceutical composition may also be provided in bandages, plasters or in sutures or the like.

In a particular embodiment, the pharmaceutical composition is suitable for oral administration, parenteral administration or topical administration. For example, the pharmaceutical composition may be suitable for topical administration (e.g. ophthalmic administration, in the form of a spray, lotion, paste or drops etc.).

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administrations of subdivided doses at specific intervals.

The pharmaceutical compositions of the invention may be administered alone or in combination with other therapeutic agents, such as anti-cancer agents, anti-Von Hippel-Lindau disease agents, antibiotics, anti-inflammatory, immunosuppressive, vasoactive and/or antiseptic agents (such as anti-bacterial agents, anti-fungicides, anti-viral agents, and anti-parasitic agents). Likewise, the pharmaceutical compositions may also contain anti-inflammatory drugs, such as steroids and macrolactam derivatives.

Such additional therapeutic agents may be incorporated as part of the same pharmaceutical composition or may be administered separately.

In addition to the agents such as cyclic peptides, polynucleotides, vectors, viral vectors and pharmaceutical compositions provided by the invention, the invention also provides corresponding uses and methods of use of these agents. In all therapeutic uses and methods of the invention it will be appreciated that the methods and uses may involve the administration of one, or more than one, for example 2, 3, 4, 5, 6, 7, 8, 9 or 10 different cyclic peptides according to the invention, polynucleotides according to the invention, vectors according to the invention, viral vectors according to the invention, cells according to the invention or pharmaceutical compositions of the invention, for example as particular combinations of these agents may have particularly useful therapeutic effects. The uses and methods may also involve the use of different combinations of types of agent, for example may involve the administration of a cyclic peptide of the invention, and a viral vector of the invention, for instance.

For example, the invention provides one or more of the cyclic peptides according to the invention, the polynucleotides according to the invention, the vectors according to the invention, the viral vectors according to the invention, the cells according to the invention or the pharmaceutical compositions of the invention or combination thereof, for use in medicine, for example for use in the treatment of prevention of disease a disorder or a condition.

The invention also provides one or more of the cyclic peptides according to the invention, the polynucleotides according to the invention, the vectors according to the invention, the viral vectors according to the inventions, the cells according to the invention or the pharmaceutical compositions of the invention or combination thereof, for use in the treatment or prevention of cancer. The cancer may be a solid cancer or may be a non-solid cancer, for example a blood cancer.

Preferably the cancer is a cancer:
that experiences a hypoxic environment and requires the typical hypoxia response for maintenance;
that are treatable or preventable by inhibition of dimerization of HIF-1a with HIF1-b; and/or
in which it is desirable to repress hypoxia induced gene expression.

Preferably the cancer is a solid cancer.

In some embodiments, the cancer is selected from the group comprising or consisting of:
acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholanglocarcinoma), Bladder cancer, Bone tumor, osteosarcoma/malignant fibrous histiocytoma, Brainstem glioma, Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Breast cancer, Bronchial adenomas/carcinoids, Burkitt's lymphoma, Carcinoid tumor, childhood, Carcinoid tumor, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Ependymoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemias, Leukaemia, acute lymphoblastic (also called acute lymphocytic leukaemia), Leukaemia, acute myeloid (also called acute myelogenous leukemia), Leukaemia, chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelodysplastic syndromes Myelodysplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma, Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma—see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).

The invention also provides one or more of the cyclic peptides according to the invention, the polynucleotides according to the invention, the vectors according to the invention, the viral vectors according to the invention, the cells according to the invention or the pharmaceutical compositions of the invention or combination thereof, for use in the treatment or prevention of a disease, disorder or condition:
that experiences a hypoxic environment and requires the typical hypoxia response for maintenance;
that are treatable or preventable by inhibition of dimerization of HIF-1a with HIF1-b; and/or
in which it is desirable to repress hypoxia induced gene expression.

It will be appreciated that any of the therapeutic agents described herein, for example the cyclic peptide according to the invention, the polynucleotide according to the invention, the vector according to the invention, the viral vector according to the invention, the cell according to the invention or the pharmaceutical composition of the invention, can be formulated as a composition. For example any of the cyclic peptide according to the invention, the polynucleotide according to the invention, the vector according to the invention, the viral vector according to the invention, or the cell according to the invention can be formulated as a pharmaceutical composition. It will also be clear that any of the cyclic peptide according to the invention, the polynucleotide according to the invention, the vector according to the invention, the viral vector according to the invention, the cell according to the invention or the pharmaceutical composition of the invention can be formulated with one or more further therapeutic agents, for example one or more further anti-cancer therapeutic agents or one or more further agents for the treatment of Von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceuticals composition of the invention or combination thereof, can be administered as part of a combination therapy. For example, the cyclic peptides according to the invention, the polynucleotides according to the invention, the vectors according to the invention, the viral vectors according to the invention, the cells according to the invention or the pharmaceutical compositions of the invention or combination thereof, can be administered prior to a further therapeutic agent, for example prior to the administration of one or more further anti-cancer therapeutic agents or one or more further agents for the treatment of von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceutical compositions of the invention or combination thereof, can be administered following the administration of a further therapeutic agent, for example following the administration of one or more further anti-cancer therapeutic agents or one or more further agents for the treatment of von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceutical compositions of the invention or combination thereof, can be administered simultaneously to the administration of a further therapeutic agent, for example simultaneous to the administration of one or more further anti-cancer therapeutic agents or one or more further agents for the treatment of von Hippel-Lindau disease. The simultaneous administration may involve the administration of a single composition comprising both the cyclic peptide according to the invention, the polynucleotide according to the invention, the vector according to the invention, the viral vector according to the invention, the cell according to the invention or the pharmaceutical composition of the invention or combination thereof, and the one or more further therapeutic agents, for example one or more further anti-cancer therapeutic agents or one or more further agents for the treatment of von Hippel-Lindau disease. The simultaneous administration may instead involve the administration of separate compositions, a first composition comprising the cyclic peptide according to the invention, the polynucleotide according to the invention, the vector according to the invention, the viral vector according to the invention, the cell according to the invention or the pharmaceutical composition of the invention and a second composition comprising the one or more further therapeutic agents, for example one or more further anti-cancer therapeutic agents or one or more further agents for the treatment of von Hippel-Lindau disease.

The invention also provides a method for the treatment or prevention of a disease, disorder or condition, wherein the method comprises administration of one or more of the cyclic peptides according to the invention, the polynucleotides according to the invention, the vectors according to the invention, the viral vectors according to the invention, the cells according to the invention or the pharmaceutical compositions of the invention or combination thereof.

Preferences for the disease, peptide and other features are described elsewhere herein.

For example, by disease, disorder or condition we include the meaning of any disease, disorder or condition:
that experiences a hypoxic environment and requires the typical hypoxia response for maintenance;
that are treatable or preventable by inhibition of dimerization of HIF-1a with HIF1-b; and/or
in which it is desirable to repress hypoxia induced gene expression, Examples of such diseases, disorders and conditions includes tumours, cancers including solid cancers and blood cancers, and von Hippel-Lindau disease.

Accordingly, the invention provides a method for the treatment or prevention of a disease, disorder or condition:
that experiences a hypoxic environment and requires the typical hypoxia response for maintenance;
that are treatable or preventable by inhibition of dimerization of HIF-1a with HIF1-b; and/or
in which it is desirable to repress hypoxia induced gene expression.
   wherein the method comprises administration of one or more of the cyclic peptides according to the invention, the polynucleotides according to the invention, the vectors according to the invention, the viral vectors according to the invention, the cells according to the invention or the pharmaceutical compositions of the invention.

The invention also provides a method for the treatment or prevention of cancer or a tumour, for example a solid cancer or solid tumour, wherein the method comprises administration of one or more of the cyclic peptides according to the invention, the polynucleotides according to the invention, the vectors according to the invention, the viral vectors according to the invention, the cells according to the invention or the pharmaceutical compositions of the invention or combination thereof.

The invention also provides:
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in medicine;
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in the treatment or prevention of a disease, disorder or condition:
   that experiences a hypoxic environment and requires the typical hypoxia response for maintenance;
that are treatable or preventable by inhibition of dimerization of HIF-1a with HIF1-b; and/or
in which it is desirable to repress hypoxia induced gene expression; and
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in for use in the treatment or prevention of cancer, for example a solid cancer or tumour.

The invention also provides a fluorescent probe wherein the fluorescent probe is a cyclic peptide according to the invention wherein X3 is Cou. In some preferred embodiments the fluorescent probe has a sequence of:

[SEQ ID NO: 39]
Cys leu leu cou val tyr.

The skilled person will also understand that various components and agents of the present invention lend themselves to being provided as part of a kit, or kit of parts. For example in one embodiment the invention provides a kit comprising one or more of:
a cyclic peptide according to the invention;
a polynucleotide according to the invention;
a nucleic acid vector according to the invention;
a cell according to the invention;
a viral vector according to the invention;
a pharmaceutical composition according to the invention; or
a fluorescent probe according to the invention.

The kit may comprise any number of these agents, for example the kit may comprise 1, 2, 3, 4, 5, 6, 7, 8, 9, 10 or more than 10 different cyclic peptides of the invention; and/or may comprise 1, 2, 3, 4, 5, 6, 7, 8, 9, 10 or more than 10 polynucleotides of the invention; for example may comprise 1, 2, 3, 4, 5, 6, 7, 8, 9, 10 or more than 10 different nucleic acid vectors of the invention; for example may comprise 1, 2, 3, 4, 5, 6, 7, 8, 9, 10 or more than 10 different cells of the invention; for example may comprise 1, 2, 3, 4, 5, 6, 7, 8, 9, 10 or more than 10 different viral vectors of the invention; for example may comprise 1, 2, 3, 4, 5, 6, 7, 8, 9, 10 or more than 10 different pharmaceutical compositions of the invention.

For example, in one embodiment the kit is suitable for determining the most appropriate cyclic peptide therapy to treat a particular tumour.

The kit may also comprise appropriate control cells and reagents.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. To exemplify how the preferences and options described throughout can be combined, the invention provides:
a cyclic peptide of sequence C X₁ X₂ X₃ X₄ X₅
   [Formula 1]
   [SEQ ID NO: 1]
wherein the cyclic peptide is capable of binding to HIF-1, optionally capable of binding to recombinantly expressed PAS-B domain of HIF-1α, and
wherein the cyclic peptide prevents or reduces the hypoxia induced expression from a promoter that comprises one or more hypoxia-responsive elements under hypoxic conditions;
   a method of treating cancer wherein the method comprises administering a pharmaceutical composition, wherein the pharmaceutical composition comprises a cyclic peptide of sequence C X₁ X₂ X₃ X₄ X₅ [Formula 1]
[SEQ ID NO: 1]
and wherein the cyclic peptide disrupts the interaction between HIF-1a and HIF-1b, optionally wherein the interaction is assessed by proximity ligation assay, optionally in MCF-7 cells.

Sequences

| SEQ ID NO: | Sequence | Notes |
|---|---|---|
| SEQ ID NO: 1 | C X₁ X₂ X₃ X₄ X₅ | Consensus sequence |
| SEQ ID NO: 2 | Cys leu leu Cha val tyr | |
| SEQ ID NO: 3 | Cys leu leu h-phe val tyr | |
| SEQ ID NO: 4 | Cys leu leu phe(4-I) val tyr | |
| SEQ ID NO: 5 | Cys leu leu phe(4-Cl) val tyr | |
| SEQ ID NO: 6 | Cys leu leu d-phe val tyr | |
| SEQ ID NO: 7 | Cys leu leu Nal val tyr | |
| SEQ ID NO: 8 | Cys leu leu phe(4-Br) val tyr | |
| SEQ ID NO: 9 | Cys leu leu phe(4-Ph) val tyr | |
| SEQ ID NO: 10 | Cys leu leu phe(CF₃) val tyr | |
| SEQ ID NO: 11 | Cys leu leu phe(CF₃) val PaI | |
| SEQ ID NO: 12 | Cys leu leu phe(CF₃) val Cha | |
| SEQ ID NO: 13 | Cys leu leu phe(CF₃) val phe(4-Ph) | |
| SEQ ID NO: 14 | Cys leu leu phe(CF₃) val d-tyr | |
| SEQ ID NO: 15 | Cys leu leu phe(CF₃) val Phg | |
| SEQ ID NO: 16 | Cys leu leu phe(CF₃) val phe(4-CF3) | |
| SEQ ID NO: 17 | Cys leu leu phe(CF₃) val phe | |
| SEQ ID NO: 18 | Cys leu leu phe(CF₃) val Tyr(me) | |
| SEQ ID NO: 19 | Cys leu leu phe(CF₃) val phe(4-I) | |
| SEQ ID NO: 20 | Cys leu leu phe(CF₃) val h-phe | |
| SEQ ID NO: 21 | Cys leu leu phe(CF₃) val phe(NO₂) | |
| SEQ ID NO: 22 | Cys leu leu phe(CF₃) val Nal | |
| SEQ ID NO: 23 | Cys leu leu phe(CF₃) val phe(4-F) | |
| SEQ ID NO: 24 | Cys leu leu phe(CF₃) val phe(4-CI) | |
| SEQ ID NO: 25 | Cys leu leu phe(CF₃) val phe(4-Br) | |
| SEQ ID NO: 26 | Cys leu leu phe(CF₃) Abu phe(4-Br) | |
| SEQ ID NO: 27 | Cys leu leu phe(CF₃) Aib phe(4-Br) | |
| SEQ ID NO: 28 | Cys leu leu phe(CF₃) n-leu phe(4-Br) | |
| SEQ ID NO: 29 | Cys leu leu phe(CF₃) Prop phe(4-Br) | |
| SEQ ID NO: 30 | Cys leu leu phe(CF₃) leu phe(4-Br) | |
| SEQ ID NO: 31 | Cys leu leu phe(CF₃) ile phe(4-Br) | |
| SEQ ID NO: 32 | Cys leu val phe(CF₃) ile phe(4-Br) | |
| SEQ ID NO: 33 | Cys leu Aib phe(CF₃) ile phe(4-Br) | |
| SEQ ID NO: 34 | Cys leu h-leu phe(CF₃) ile phe(4-Br) | |
| SEQ ID NO: 35 | Cys leu n-val phe(CF₃) ile phe(4-Br) | |
| SEQ ID NO: 36 | Cys leu Prop phe(CF₃) ile phe(4-Br) | |
| SEQ ID NO: 37 | Cys leu ile phe(CF₃) ile phe(4-Br) | |
| SEQ ID NO: 38 | Cys leu n-leu phe(CF₃) ile phe(4-Br) | |
| SEQ ID NO: 39 | Cys leu leu cou val tyr | |
| SEQ ID NO: 40 | Cys lys n-leu phe(CF₃) ile phe(4-Br) | |
| SEQ ID NO: 41 | Cys glu n-leu phe(CF₃) ile phe(4-Br) | |
| SEQ ID NO: 42 | h-cys leu leu phe val tyr | |
| SEQ ID NO: 43 | d-cys leu leu phe val tyr | |
| SEQ ID NO: 44 | Cys leu leu phe val tyr | |
| SEQ ID NO: 45 | Ser leu leu phe val tyr | Comparative purposes |
| | | only |
| SEQ ID NO: 46 | Thr leu leu phe val tyr | Comparative purposes |
| | | only |
| SEQ ID NO: 47 | Met leu leu phe val tyr | Comparative purposes |
| | | only |
| SEQ ID NO: 48 | Tp leu leu phe val tyr | Comparative purposes |
| | | only |
| SEQ ID NO: 49 | Ala leu leu phe val tyr | Comparative purposes |
| | | only |
| SEQ ID NO: 50 | Cys leu leu Tyr(me) val tyr | Comparative purposes |
| | | only |
| SEQ ID NO: 51 | Cys leu leu tyr val tyr | Comparative purposes |
| | | only |
| SEQ ID NO: 52 | Cys leu leu PhG val tyr | Comparative purposes |
| | | only |
| SEQ ID NO: 53 | Cys leu leu 4-Pal val tyr | Comparative purposes |
| | | only |
| SEQ ID NO: 54 | Cys leu leu phe(NO₂) val tyr | Comparative purposes |
| | | only |
| SEQ ID NO: 55 | Cys leu leu phe(4-F) val tyr | Comparative purposes |
| | | only |
| SEQ ID NO: 56 | Cys leu leu phe(4-tBu) val tyr | |
| SEQ ID NO: 57 | Cys leu leu phe(CF₃) val phe(4-tBu) | |
| SEQ ID NO: 58 | Cys leu leu phe(CF₃) n-val phe(4-Br) | |
| SEQ ID NO: 59 | Cys leu leu phe(CF₃) h-leu phe(4-Br) | |

For the avoidance of doubt, cyclic peptides of SEQ ID NO: 45-55 are included for comparative purposes only.

### FIGURE LEGENDS

FIG. 1—Assessing the activity of cyclo-CLLFVY (SEQ ID NO: 44 Cys leu leu phe val tyr) via MST shows that it binds with a Kd 36.04±3.6 μM.

FIG. 2—A) Bar Chart showing the Kds obtained when replacing cysteine with various amino acid analogues. The data shows that all analogues resulted in poorer binding than the parent compound and only thiol containing amino acids retain any binding activity. B) Bar Chart showing the affinity of Cyclo-CLLFVY for the three cysteine mutant proteins (C255A, C334A and C337A) C) Cysteine derivative MST curves. Tp=Thioproline, M=methionine, A=alanine, S=serine, T=threonine.

FIG. 3—Cysteine mutant HIF-1a Pas-B domains C255A, C334A and C337A.

FIG. 4—A) Bar Chart showing the Kds obtained when replacing phenylalanine with various amino acid analogues. B) MST traces of phenylalanine derivatives with a KD<10 μM. Error bars represent the standard deviation of n=3.

FIG. 5—A) Bar Chart showing the Kds obtained when replacing tyrosine with various amino acid analogues. B) MST traces of tyrosine derivatives with a KD<2.36 μM. Error bars represent the standard deviation of n=3.

FIG. 6—Bar Chart showing the Kds obtained upon substitution of the valine residue.

FIG. 7‥A) Bar Chart showing the Kds obtained when replacing the leucine at position 2 various amino acid analogues. B) MST traces of leucine derivatives.

FIG. 8‥MST analysis of CK-n-leu-F(CF3)IF(Br) showed that the peptide bound with a Kd of 1.66±0.3 μM and therefore that the incorporation of the lysine residue is well tolerated.

FIG. 9‥Effects of (CONC) CK-n-leu-F(CF3)IF(Br) [SEQ ID NO: 40 Cys lys NL phe(CF3) ile phe(Br)] on the heterodimerization of His-HIF-1α PAS-B domain with FLAG-HIF-1β PAS-B domain analysed by ELISA. CK-n-leu-F(CF3)IF(Br) disrupts this interaction with an IC50 of 27.7±14 μM.

FIG. 10—The parent peptide CLLFVY was found to inhibit DNA binding with an IC50 of 76.5=8.0 μM and the peptide CLLF(CF3)IF(Br) [SEQ ID NO: 31 Cys leu leu phe(CF3) ile phe(Br)] was found to inhibit FAM-HRE binding with an IC50 of 11.76±1.1 μM demonstrating that the improvements seen during the SAR study translates to increased potency when inhibiting DNA binding.

FIG. 11—A) Structure of cyclic peptide that incorporates a coumaryl motif at the phenylalanine position CLL-Cou-VY [SEQ ID NO: 39 Cys leu leu cou val tyr]. B) MST analysis of CLL-Cou-VY. C) The peptide was seen to be internalized in HeLa cells upon visualization with PMT detection at 405 nm. D) HIF-1α immunofluorescence was then used to show that the compound is preventing HIF-1α nuclear localization. E) HIF-1α immunofluorescence under control conditions showing that without treatment HIF-1a localizes to the nucleus.

FIG. 12—Fluorescent Polarization Assays

HIF-1 three-domain protein was thawed on ice and diluted to 150 nM in HIF FP buffer. Compounds for analysis were serially diluted in DMSO to give 10× stocks. The compounds (10 μL, 100% DMSO) were then added to the protein (80 μL). The samples were then incubated at room temperature for 30 minutes. FAM-HRE (10 μL, 20 nM) was then added and the samples loaded into a 384 well solid black non-binding microplate (Corning, USA). The fluorescence polarization of each well was then measured using gain and focus settings optimized by the used software set to a signal of 400 mP on a control well.

FIG. 13—A Panc HRE readout cell line was then used to show that CK-n-leu-F(CF3)IF(Br) [SEQ ID NO: 40 Cys lys n-leu phe(CF3) ile phe(Br)] inhibited the expression of EYFP and therefore inhabits HIF-1a driven transcription in this cell line. B) A Cytotox-Glo assay was used to assess the toxicity of CK-n-leu-F(CF3)IF(Br) and showed that the peptide was not toxic at doeses 50 uM. C) A U2OS HRE readout assay was used to show that the peptide CL-n-leu-F(CF3)IF(Br) [SEQ ID NO: 35 Cys leu n-leu phe(CF3) He phe(Br)] inhibited the expression of EYFP and therefore inhabits HIF-1a driven transcription in this cell line.

### EXAMPLES

### Example 1

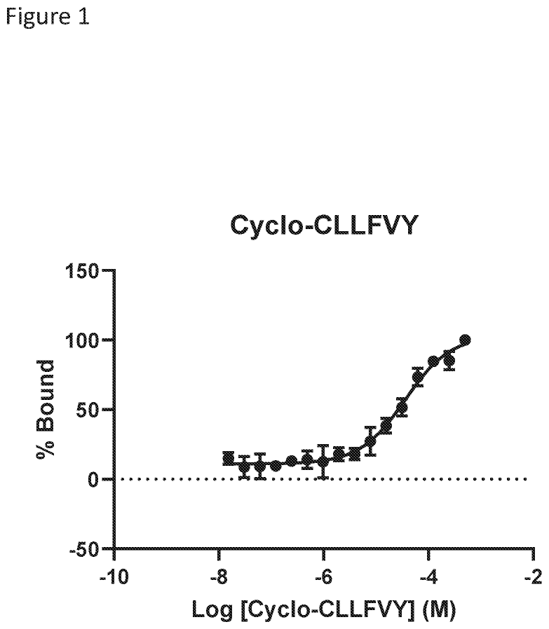

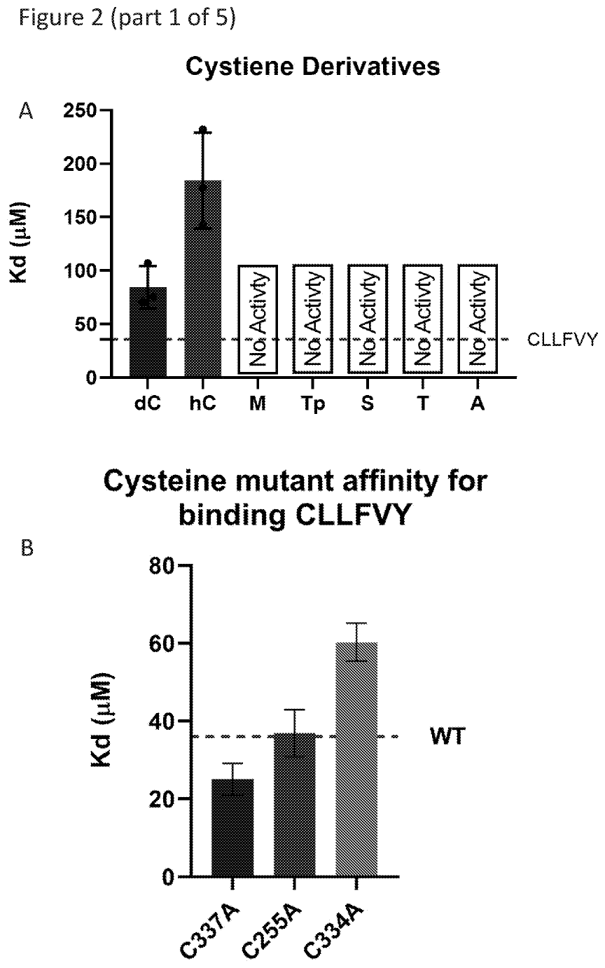

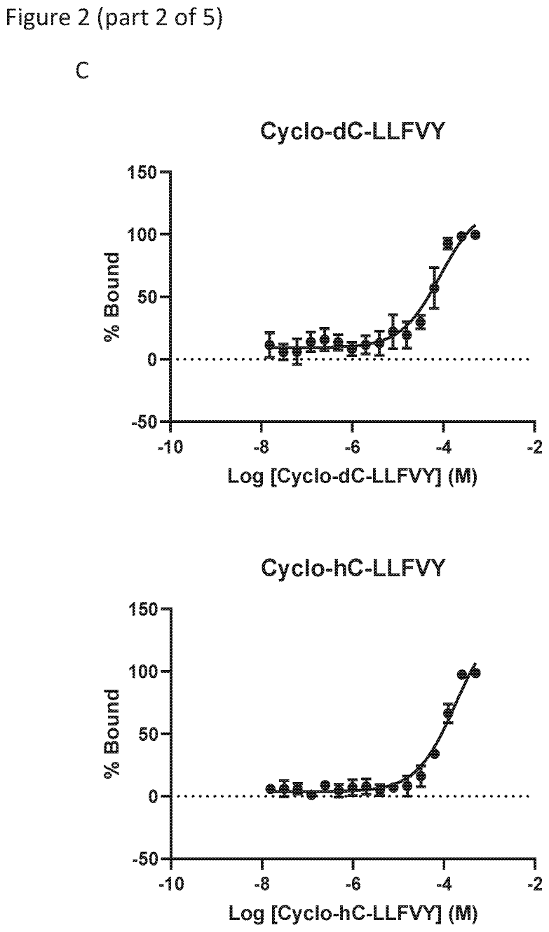

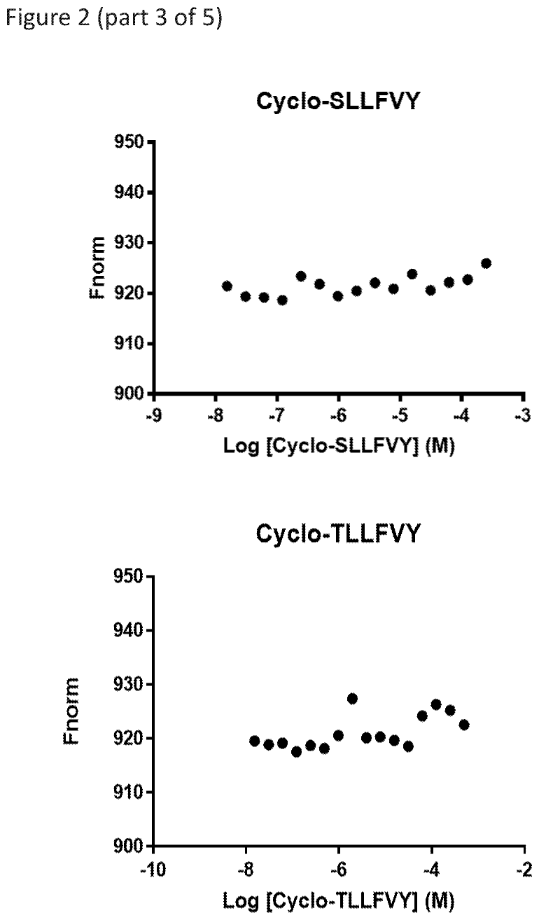

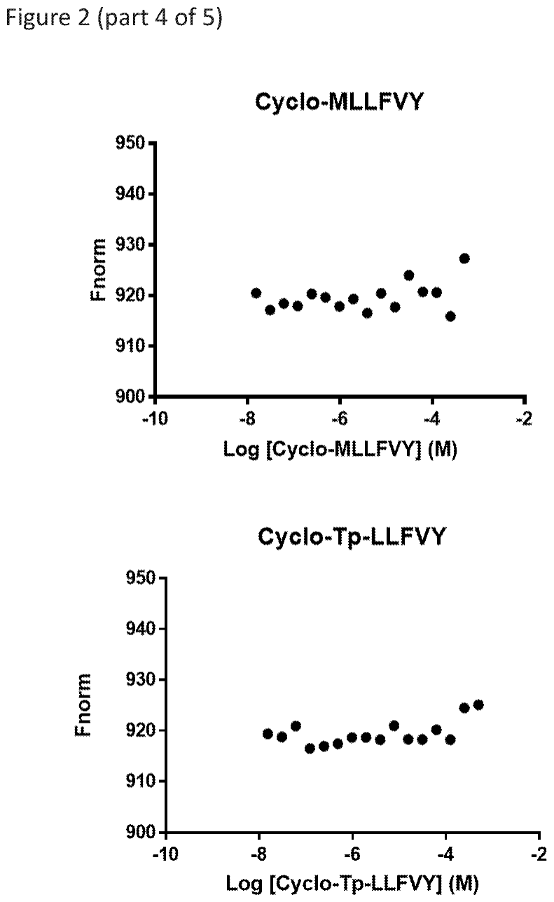

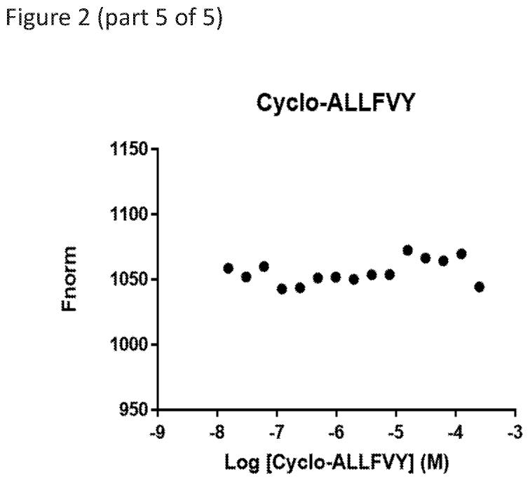

Cyclo-CLLFVY was obtained by Fmoc solid phase peptide synthesis, and its binding affinity for the HIF-1α PAS B domain determined via microscale thermophoresis (MST). The peptide was found to bind with a Kd of 36.04±3.6 μM (FIG. 1).

A series of modifications were then carried out whereby the amino acids of the peptide were sequentially replaced with a variety of natural and unnatural amino acid analogues. The impact of these substitutions on the binding affinity was then assessed by MST.

### Modifications at the Cysteine Position

The first position investigated was the cysteine, with seven analogues substituted into this position. The affinity of the new peptides was determined by MST and the resulting KD values are given in Error!Reference source not found.

The majority of substitutions had no detectable binding activity, and none of the substitutions led to a peptide with a higher affinity than cyclo-CLLFVY. D-cysteine and homocysteine gave comparable affinities to the unsubstituted peptide.

Notably both the analogues that did show binding contained a free thiol group. This is suggestive of a key interaction between this functionality and the protein. One rationalization would be that the peptide binding is disulphide-mediated. Whilst this was thought to be unlikely as all assays were performed in the presence of the reducing agent TCEP, it was decided that the possibility would be further investigated via site-directed mutagenesis (SDM). The three cysteine residues within the HIF-1α PAS-B domain were sequentially mutated to alanine. The affinity of cyclo-CLLFVY for the resultant mutant proteins C255A, C344A and C337A was then assessed by MST.

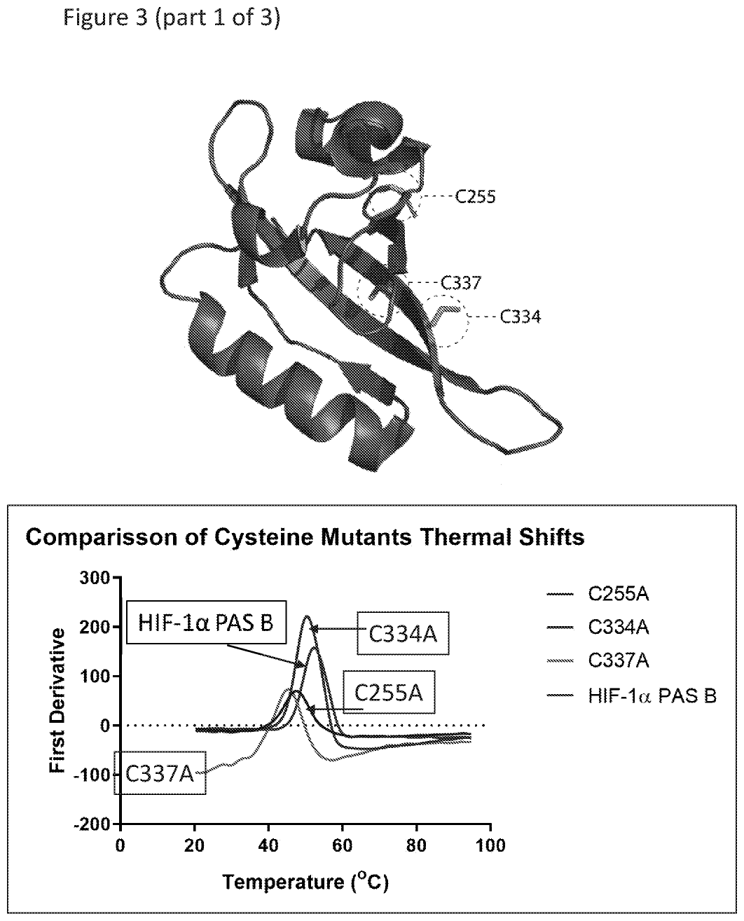

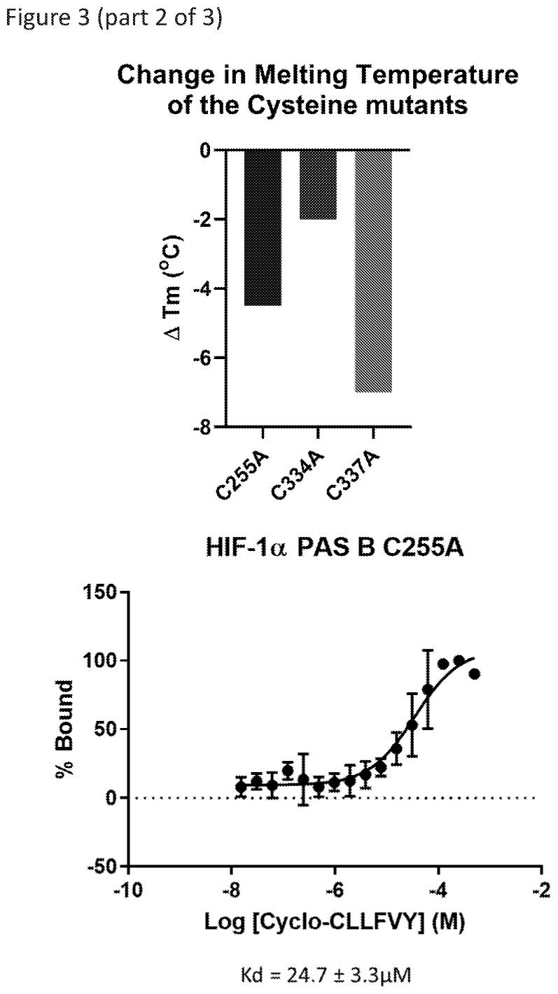

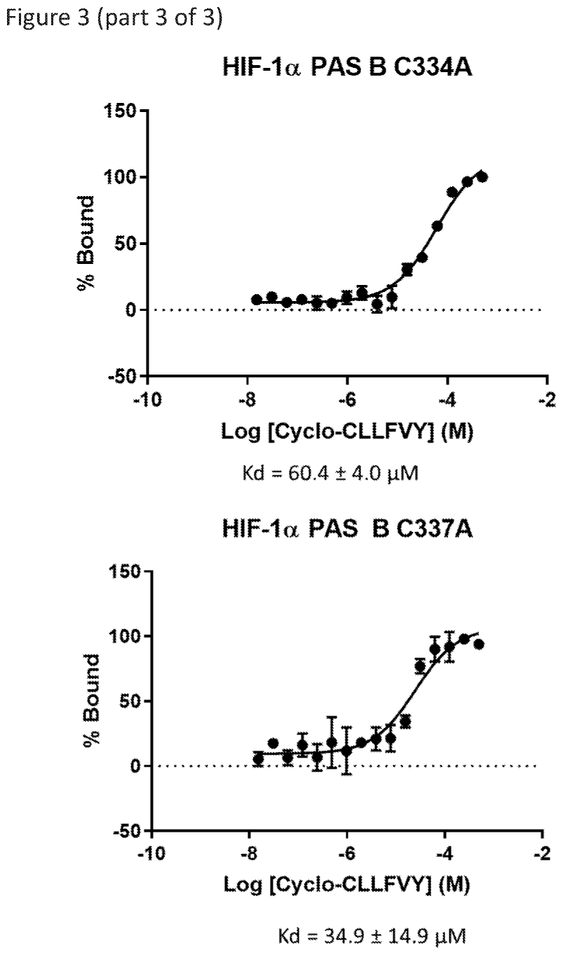

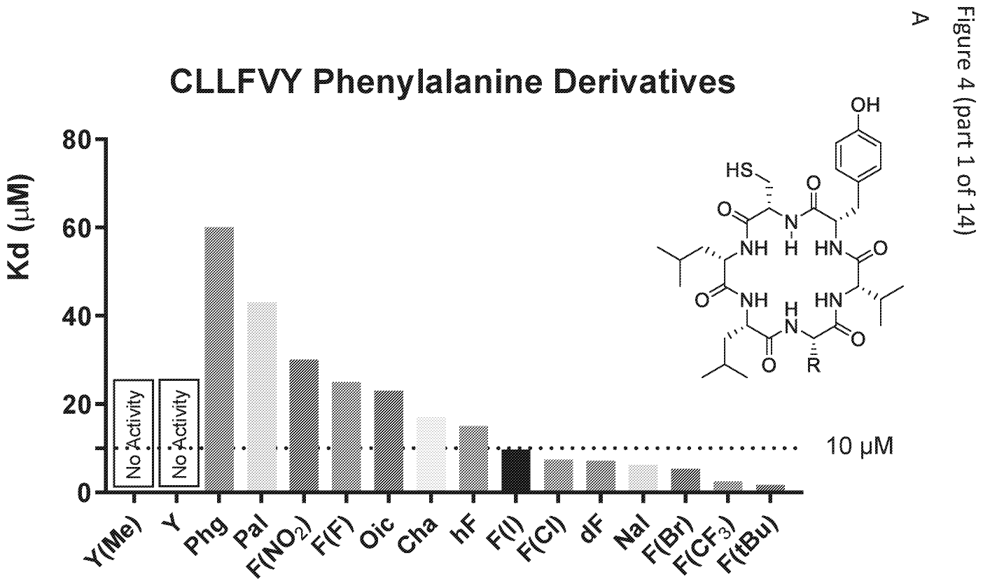

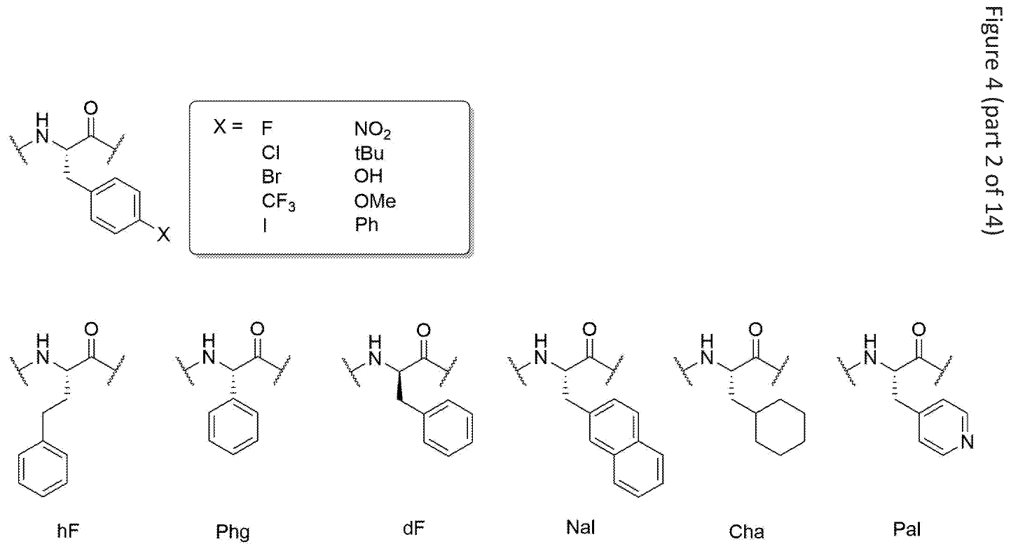

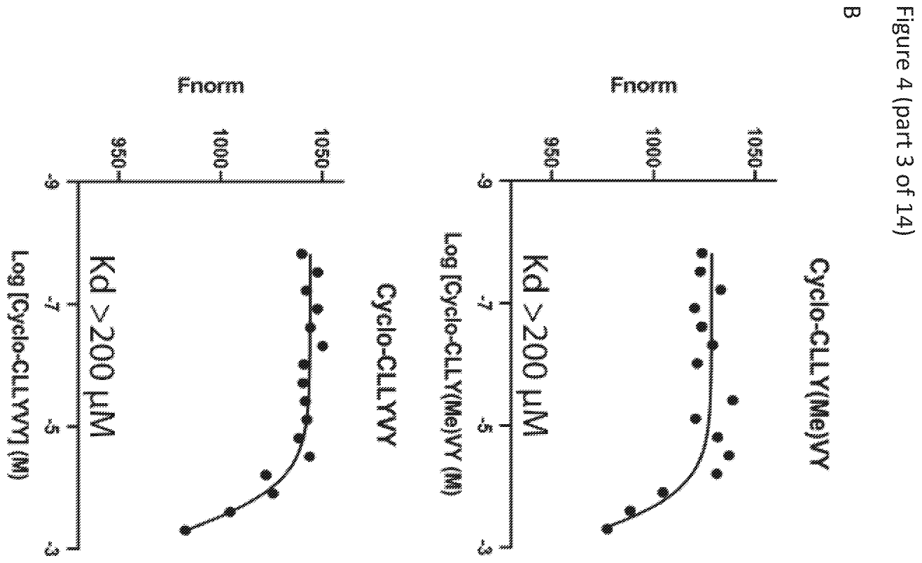

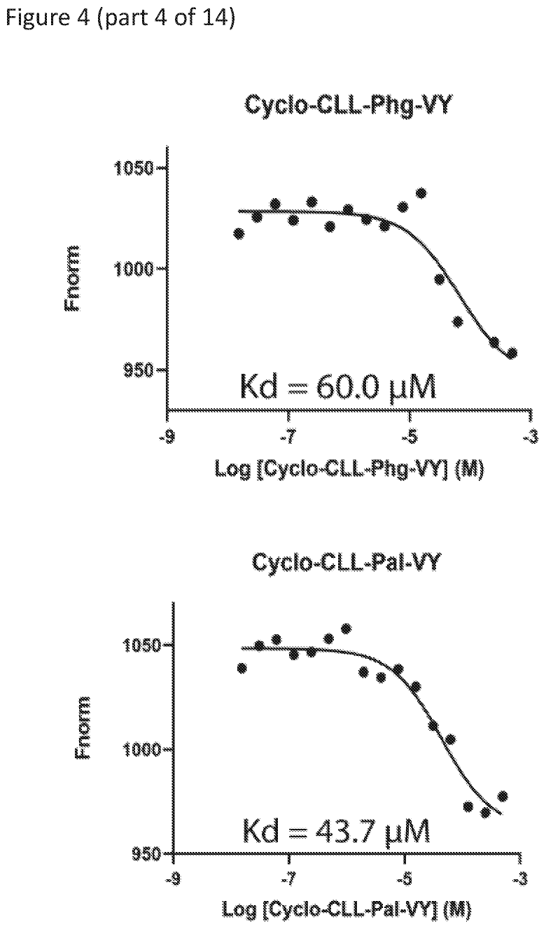

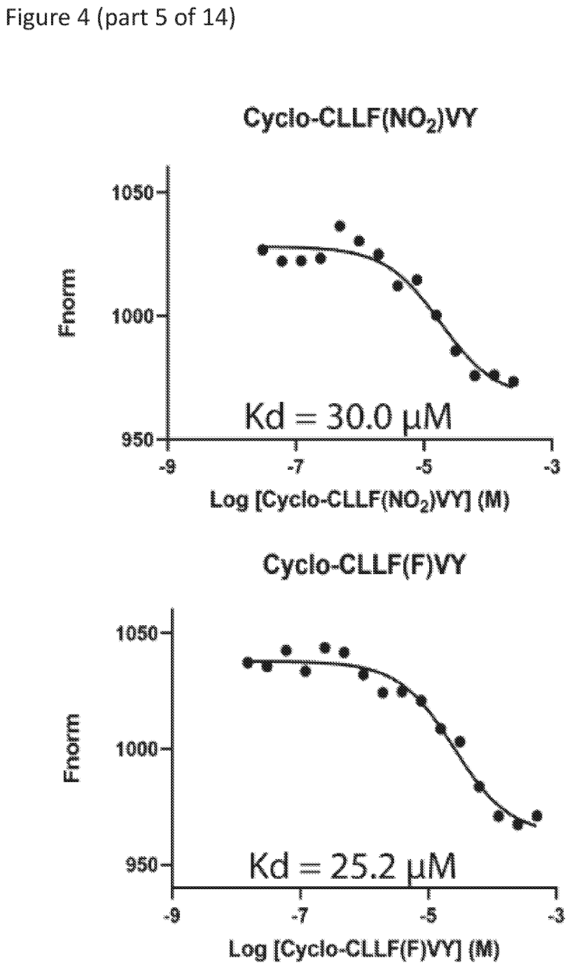

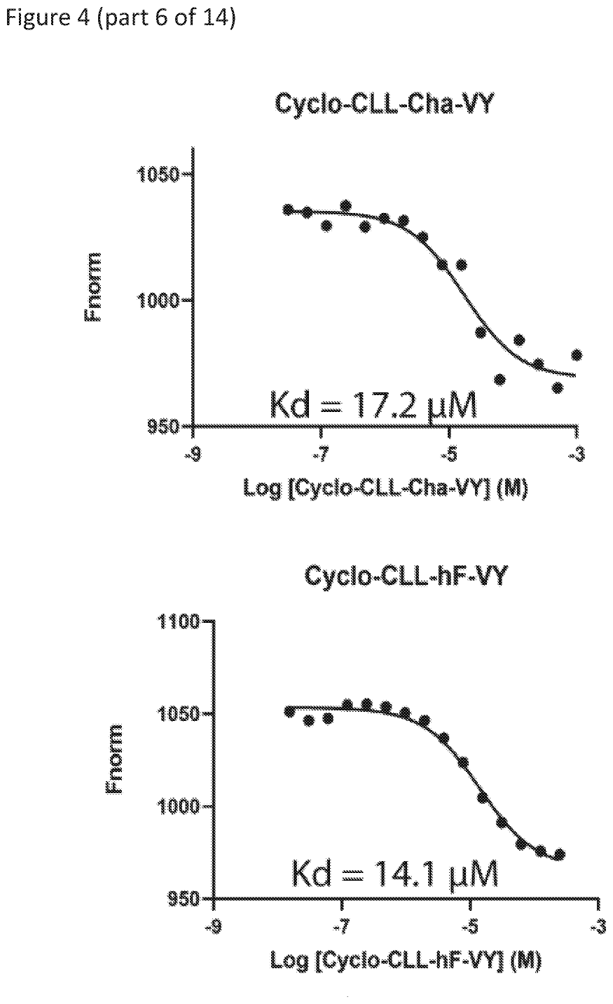

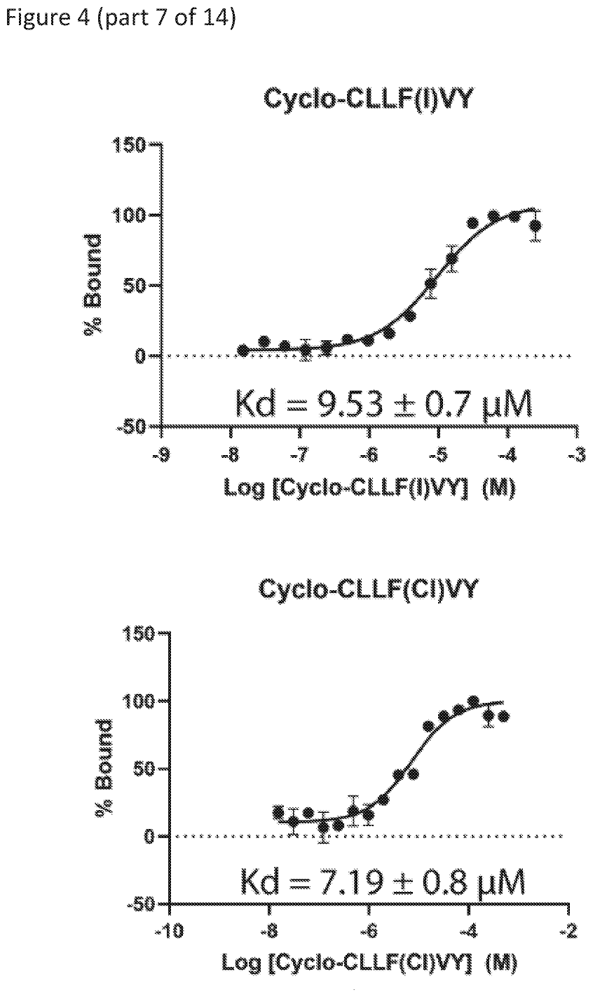

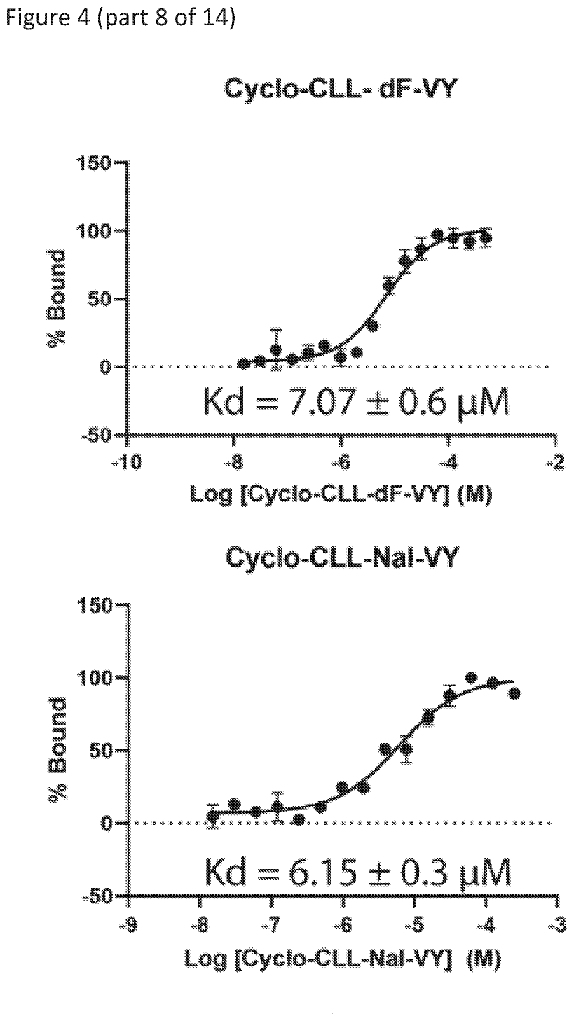

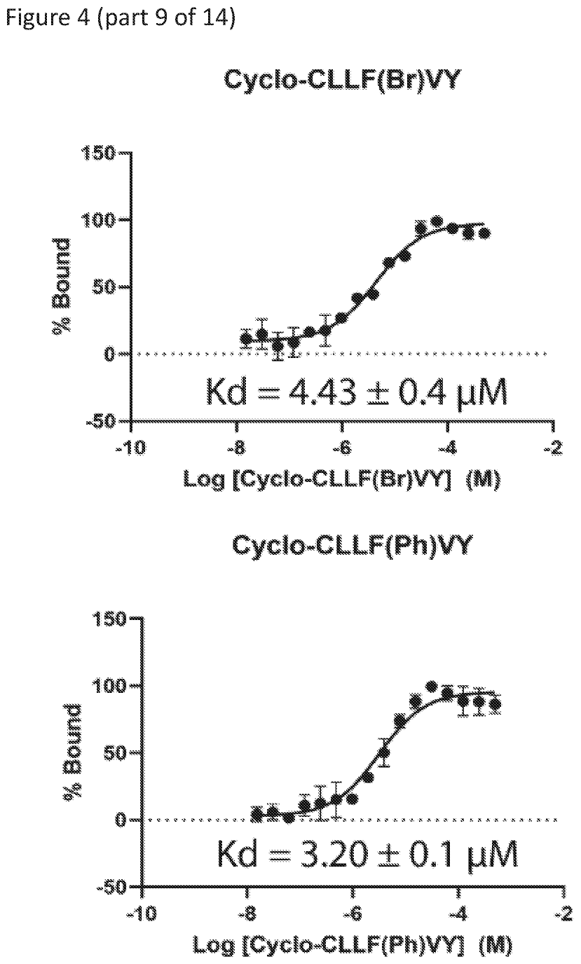

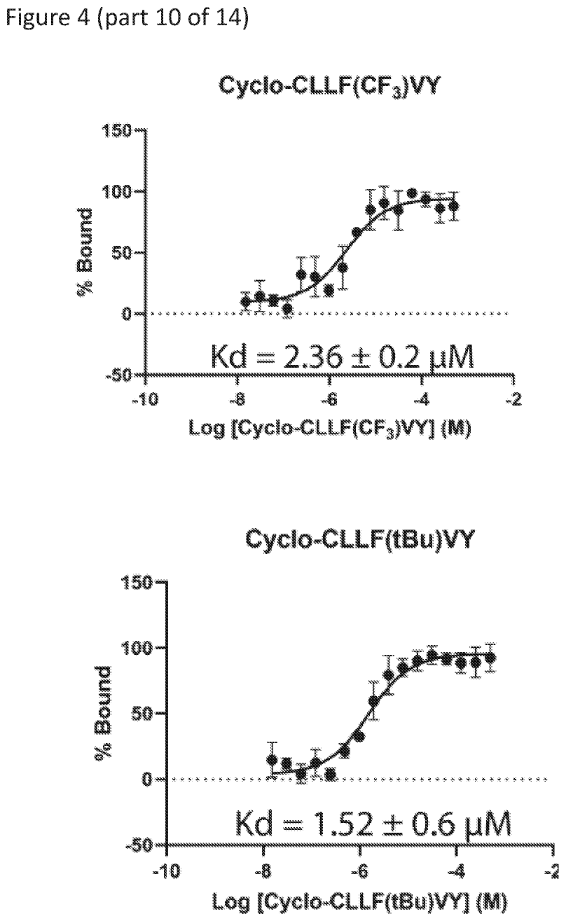

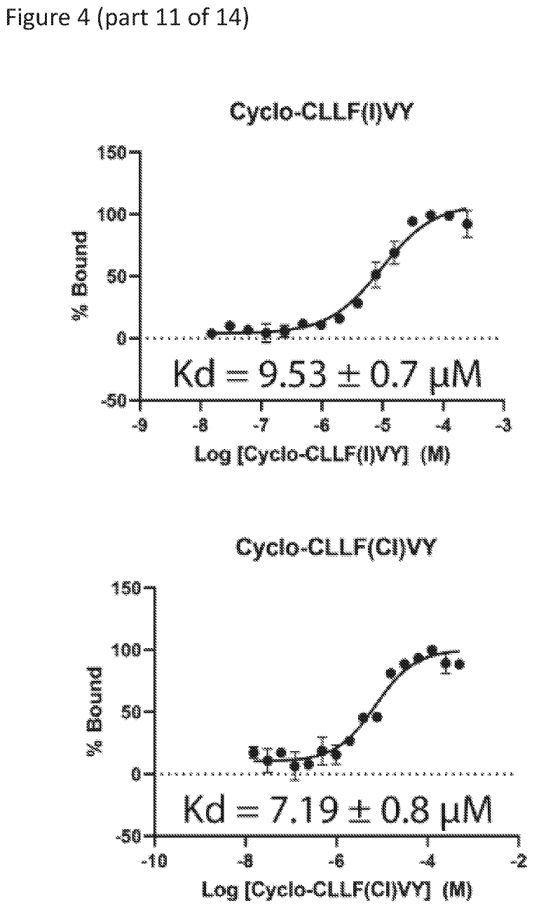

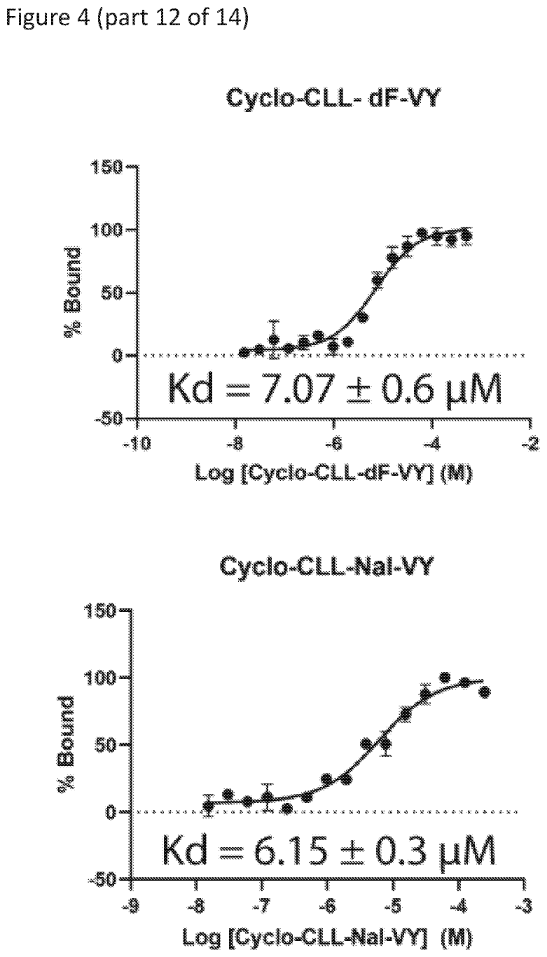

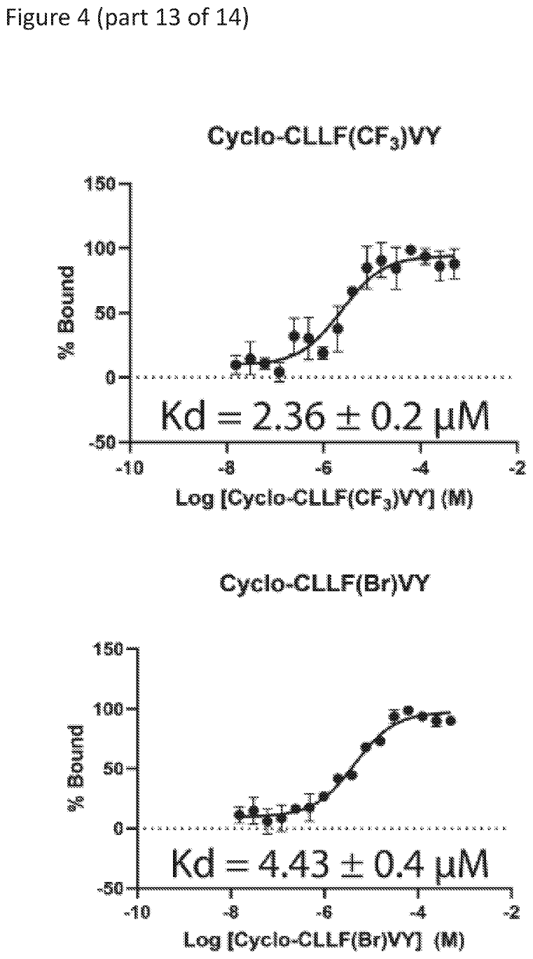

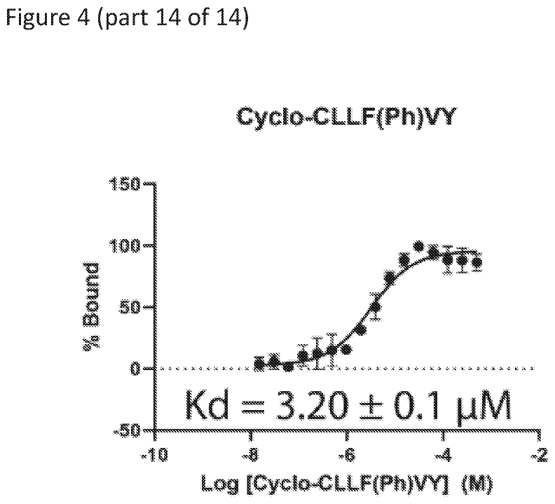

The SDM experiments show that binding activity is retained for all three mutant HIF-1α proteins. Whilst some variation is seen this is likely attributable to changes in protein stability brought about by the mutations which is corroborated by the thermal shift profiles of the three mutants (FIG. 3). If the peptide binding was disulfide mediated, then it would be expected that one of the mutant proteins would have no detectable binding activity as was seen for the derivatives lacking a thiol moiety. It was decided that as the cysteine had the best affinity and was not forming a covalent link that it would be retained, and the next position of the peptide investigated.

### Modifications at the Phenylalanine Position

A series of 16 phenylalanine analogues were synthesized and their affinity for the HIF-1α PAS-B domain determined by MST. Initially a single 16-point run was carried out for each compound after which any peptide with a Kd<10 μM was run in triplicate. The resulting Kd values are given in Error!Reference source not found.4. Within this data set we see a preference for large hydrophobic substituents (Nal, phe(4-Ph), phe(CF₃)), with phe(CF₃) yielding the best affinity with a kd of 2.36±0.2 μM, representing a 15-fold improvement in activity compared to the parent peptide.

### Modifications at the Tyrosine Position

CLLF(CF3)VY [SEQ ID NO: 10 Cys leu leu phe(CF3) val tyr] was taken into the next round of analysis.

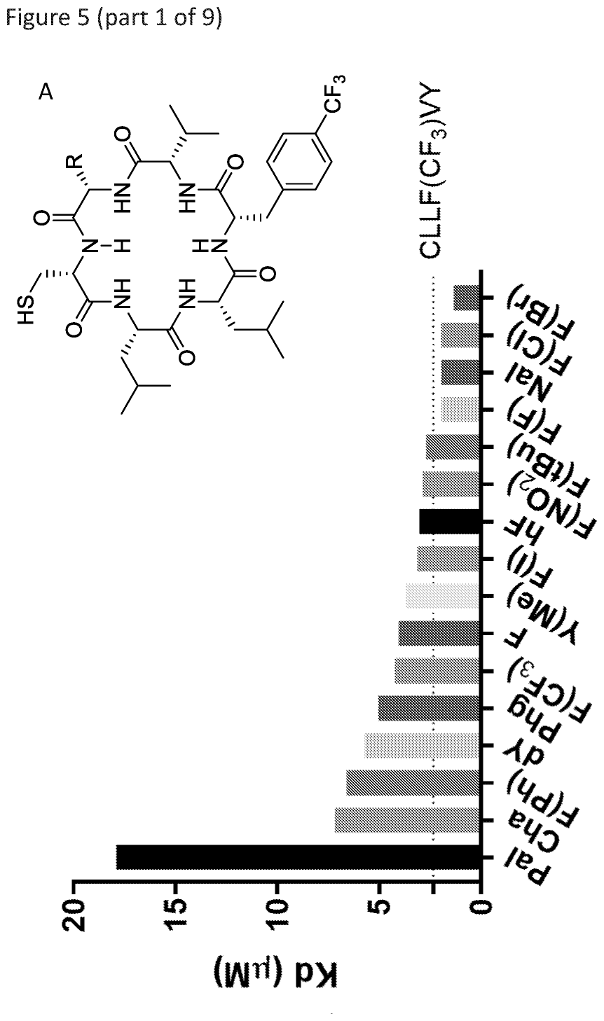

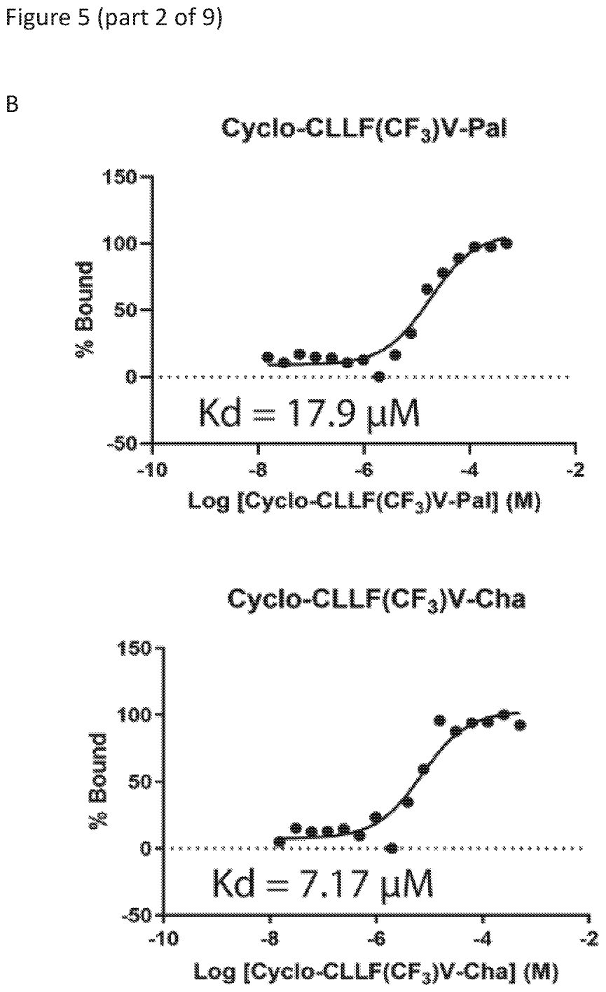

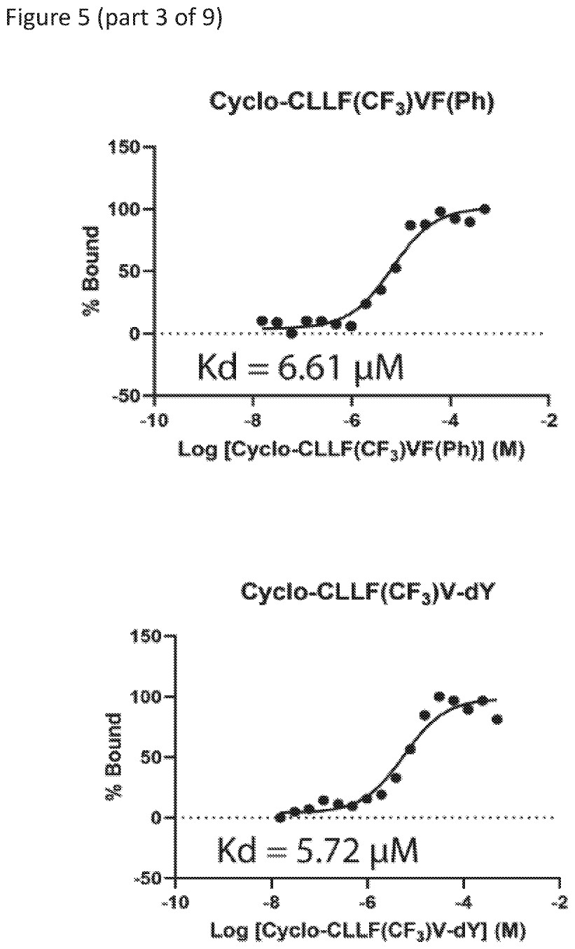

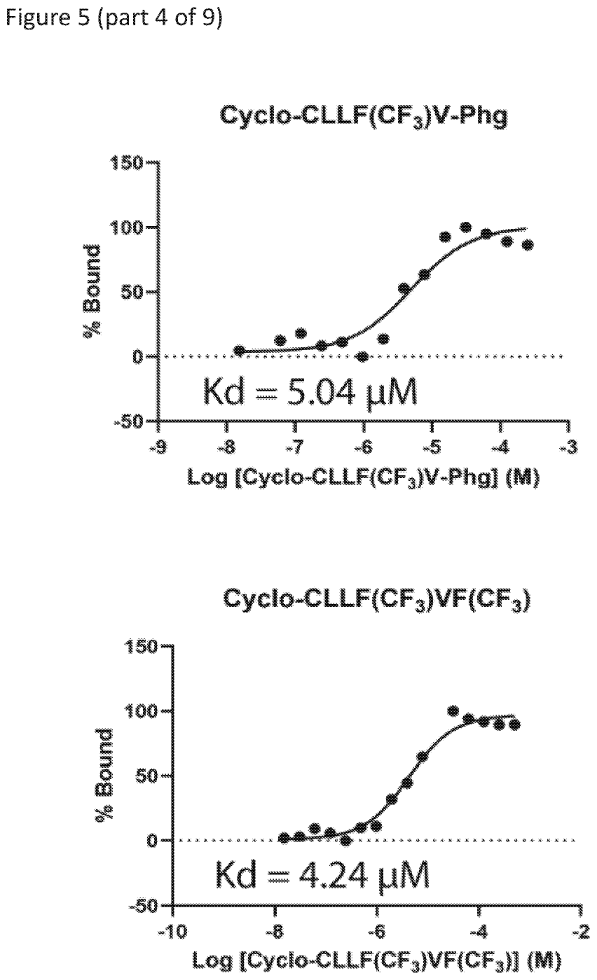

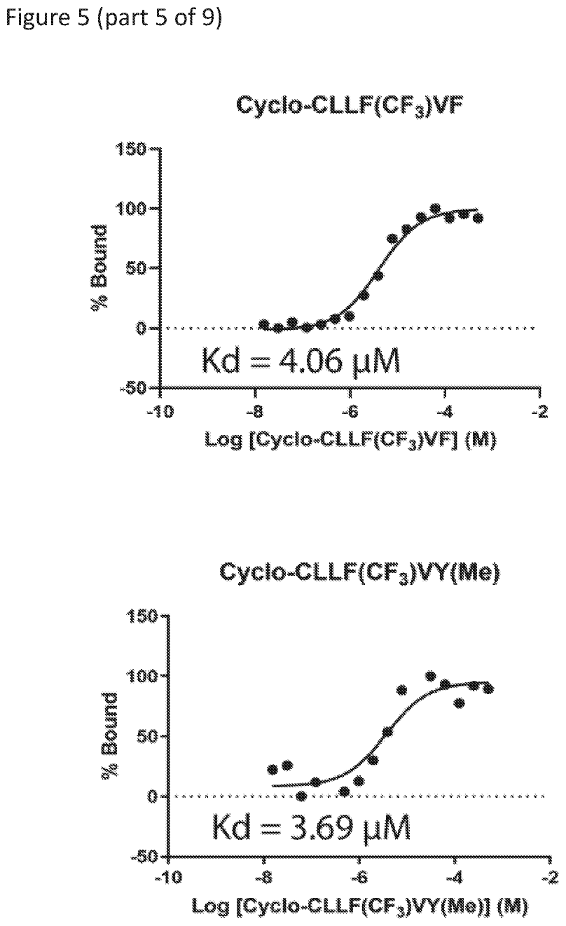

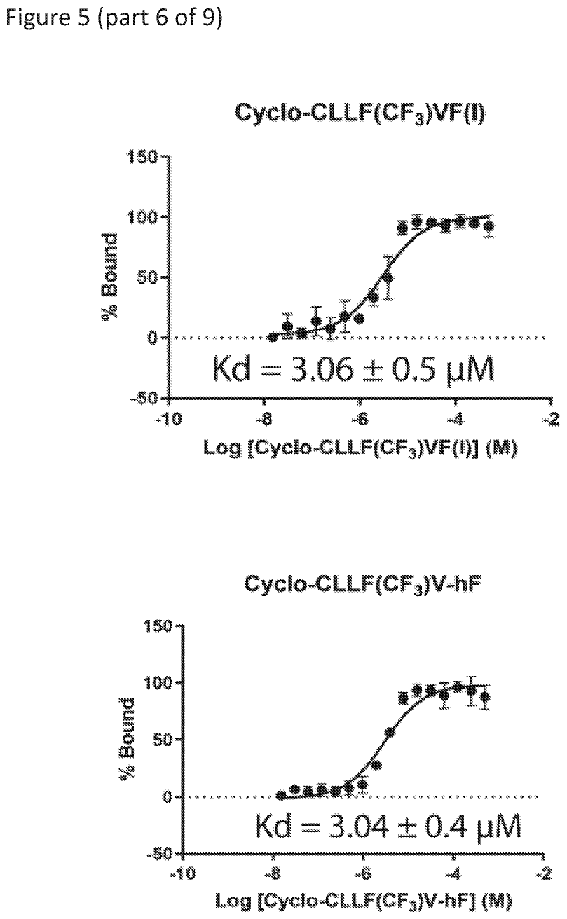

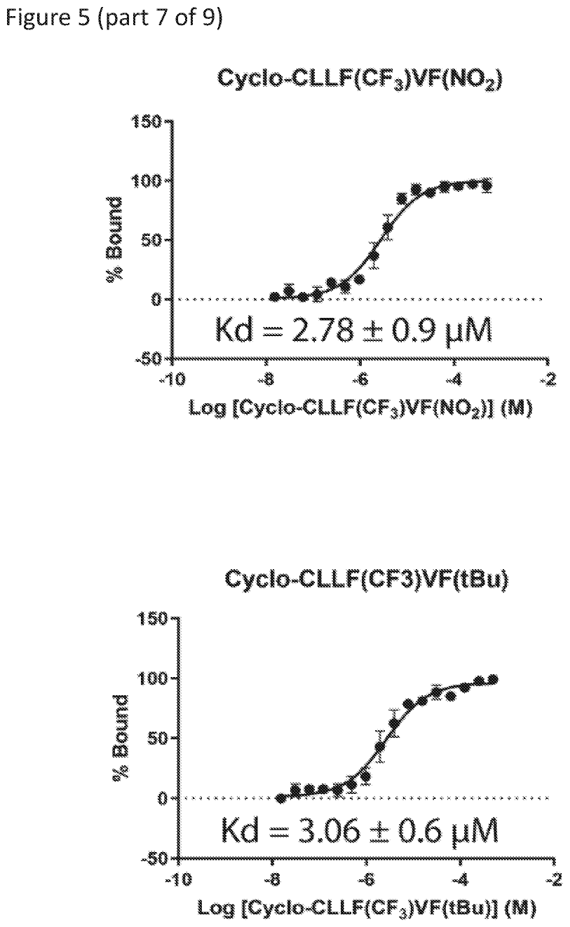

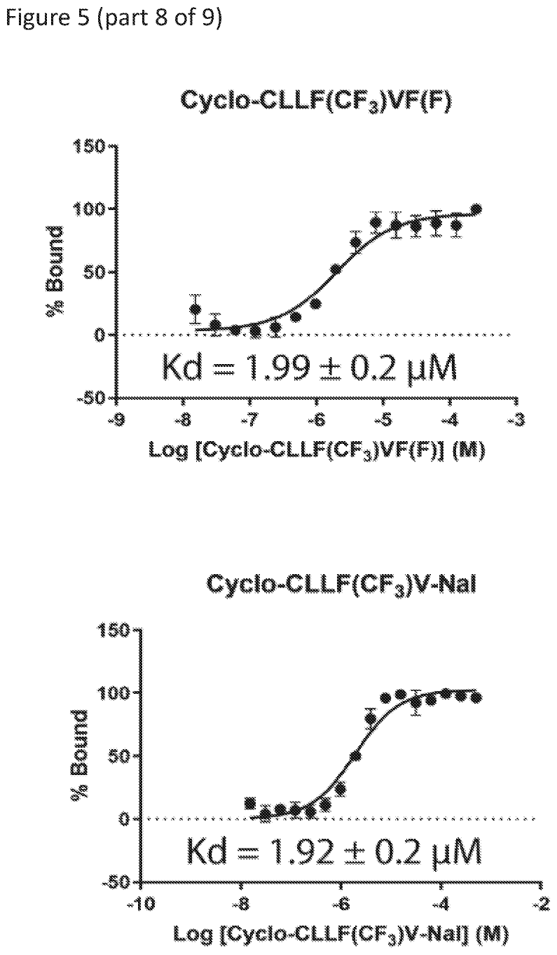

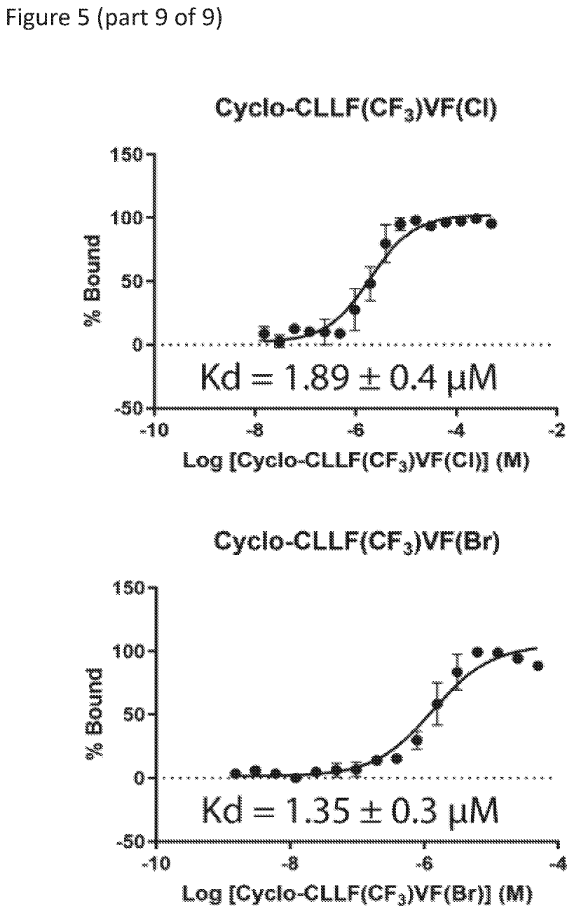

We next used the same amino acid series at the remaining aromatic position, with the exception of d-phe being replaced with d-tyr. This yielded the compound series shown in FIG. 5.

Again the peptides were initially assessed by a single MST run after which any peptide binding with an affinity greater than CLLF(CF₃)VY [SEQ ID NO: 10] was run in triplicate. This series yielded more modest changes in affinity and suggests that the tyrosine position of cyclo-CLLF(CF₃)VY is not forming a critical interaction with the HIF-1α PAS-B protein. As such there is not as a clear a trend observed in the SAR of these derivatives though it is noteworthy how more polar derivatives (F(NO₂)) are tolerated at this position as predicted through the SICLOPPS screening selecting tyrosine over phenylalanine at this position. As was seen for the phenylalanine position extended aromatic systems were well tolerated (Nal) as well as large substituents (phe(4-Br), phe(4-tBu). The amino acid resulting in the greatest affinity for binding was phe(4-Br) with a Kd of 1.35±0.3 μM and so the CLLF(CF₃)VF(Br) [SEQ ID NO: 25 Cys leu leu phe(CF₃) val phe(4-Br)] peptide was carried forward for further optimization.

### Modifications at the Valine Position

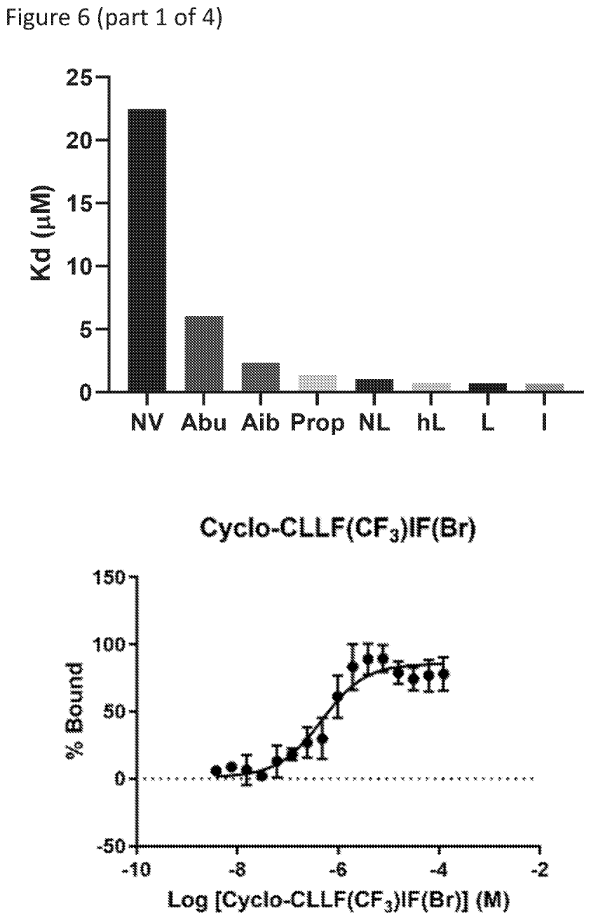

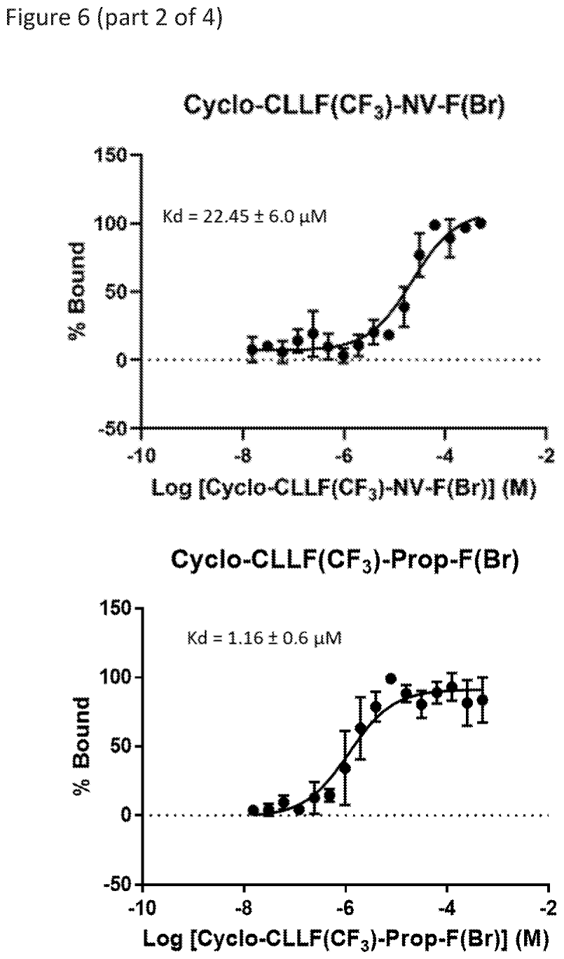

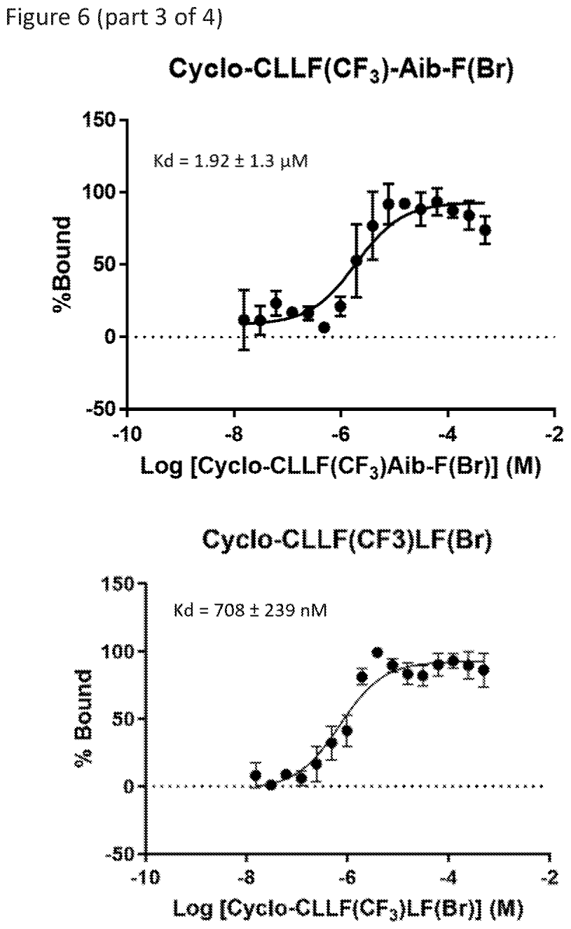

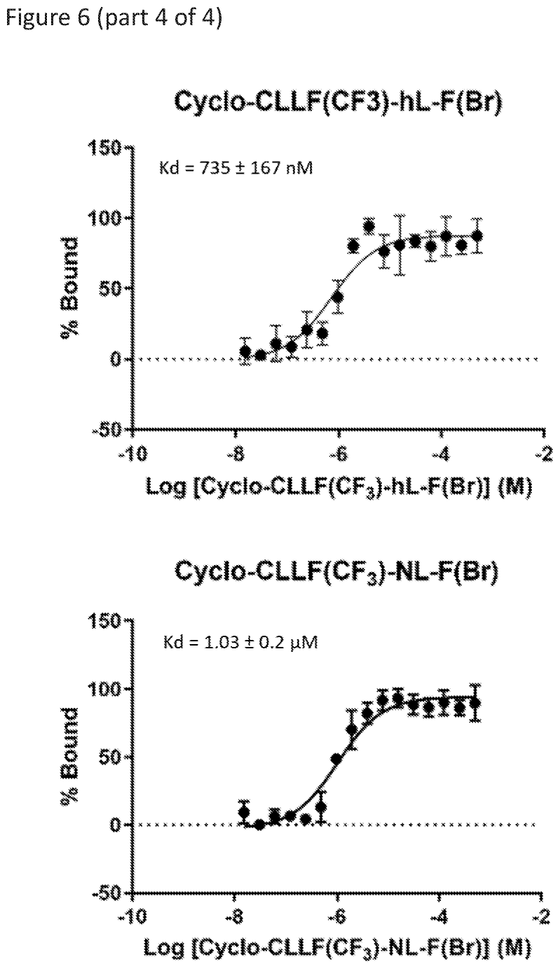

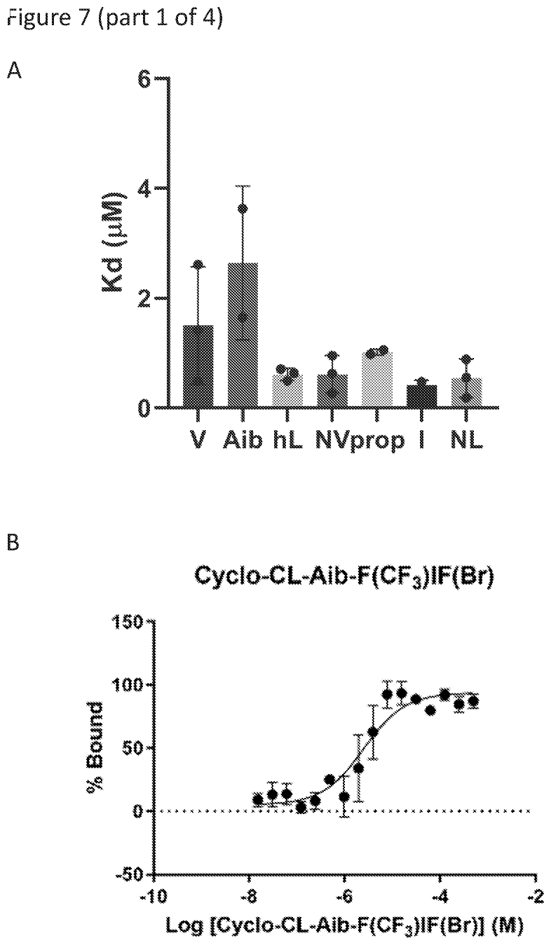

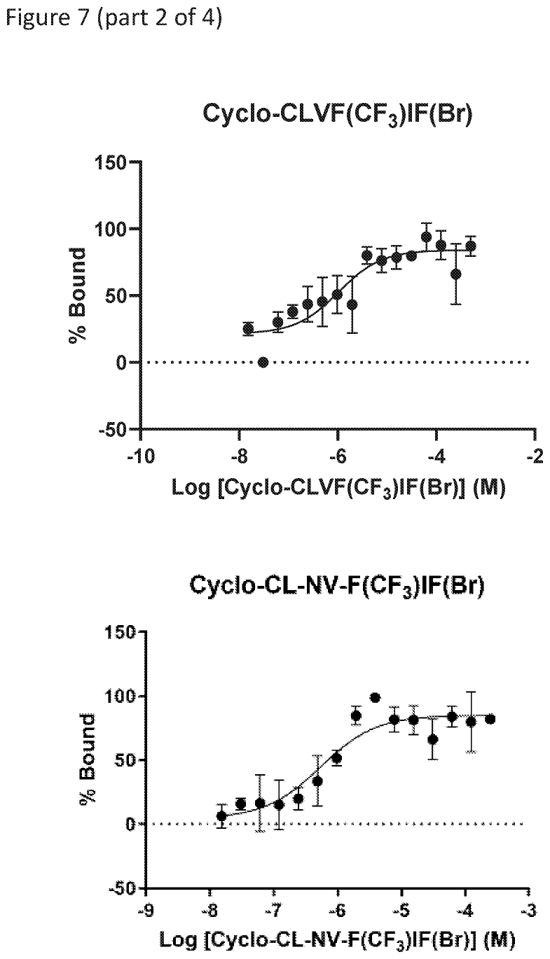

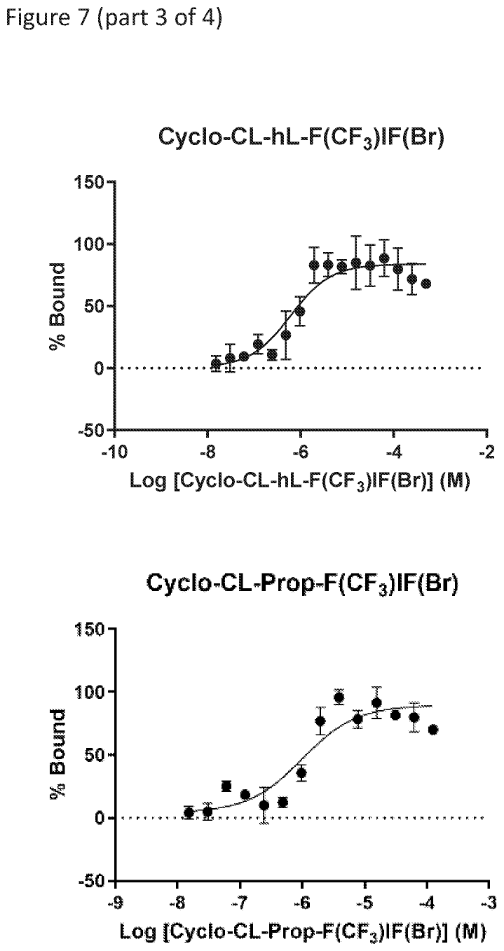

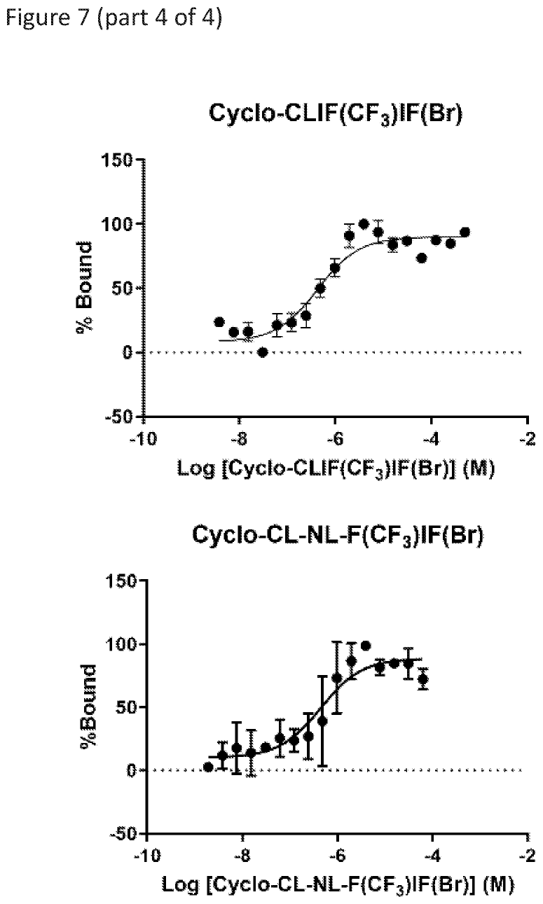

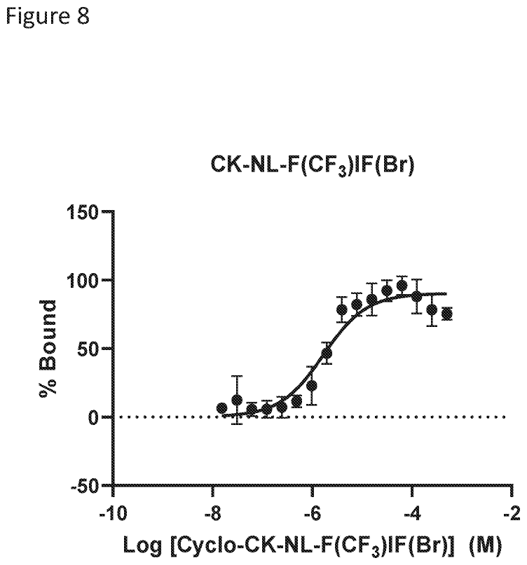

The next position investigated was the valine, a series of aliphatic amino acids were selected and substituted into this position. The resulting peptides were assessed by MST and the resulting Kds of this compound series are shown in FIG. 6. The majority of substitutions yielded minor variations in binding affinity with most of the peptides having a Kd of approximately 1 μM. A modest increase in affinity was seen upon substitution with amino acids with branched side chains with hL, L and I yielded the best affinities of the series. Isoleucine gave the best affinity and so the CLLF(CF₃)IF(Br) [SEQ ID NO: 31 Cys leu leu phe(CF₃) ile phe(4-Br)] peptide was carried forward.

### Modifications at the Leucine 2 Position

The same aliphatic derivatives (replacing leu for val) were then used to investigate the second leucine of the peptide (CLLF(CF₃)IF(Br)). Upon running the MSTs, it was noted that the error of the binding curves in this derivative series had increased. This was hypothesized to be the result of a deteriorating solubility profile, though no clear precipitation was observed upon sample preparation, there is a clear correlation between increasing C log P of the peptides and their affinity. This highlights a challenge in the development of inhibitors targeting PPIs as the relatively flat and featureless binding surfaces lead to an increasing reliance on weaker Van der Waals forces, that pushes hit compounds to become increasingly hydrophobic.

| | | |
|---|---|---|
| Peptide | Kd (μM) | CLogP |

| | |
|---|---|
| CLLFVY | 6.47 |
| CLLF(CF₃)VY | 7.36 |
| CLLF(CF₃)VF(Br) | 8.89 |
| CLLF(CF₃)IF(Br) | 9.42 |

CL-NL-F(CF₃)IF(Br) [SEQ ID NO: 38 Cys leu n-leu phe(CF₃) ile phe(4-Br)] was taken forward for further analysis.

### Incorporation of Lysine

It was noted that during SICLOPPS screening against HIF a number of the resultant hit compounds contained an amino acid bearing a positive charge in the second position immediately after the cysteine. In an attempt to improve the solubility of the CL-n-leu-F(CF₃)IF(Br) peptide its tolerance for incorporation of a positive charge was investigated. This was achieved by synthesizing the peptide CK-n-leu-F(CF₃)IF(Br) [SEQ ID NO: 40 Cys lys n-leu phe(CF₃) ile phe(4-Br)]. MST analysis of CK-n-leu-F(CF₃)IF(Br) showed that the peptide bound with a Kd of 1.66±0.3 μM and therefore that the incorporation of the lysine residue is well tolerated.

Solubility of CK-n-leu-F(CF₃)IF(Br) compound C log P=7.79

### ELISAs

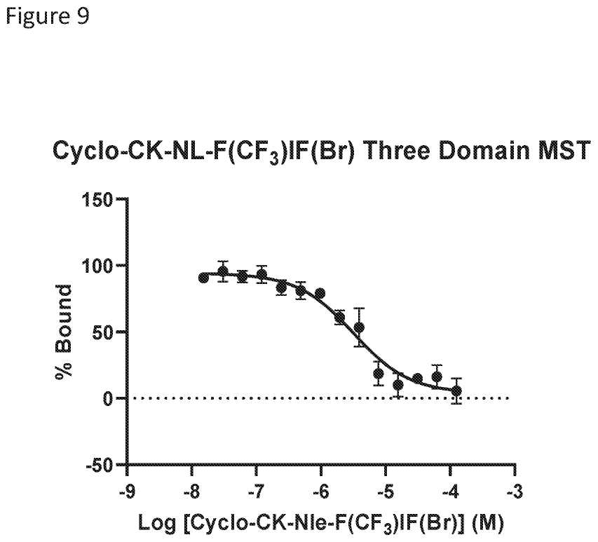

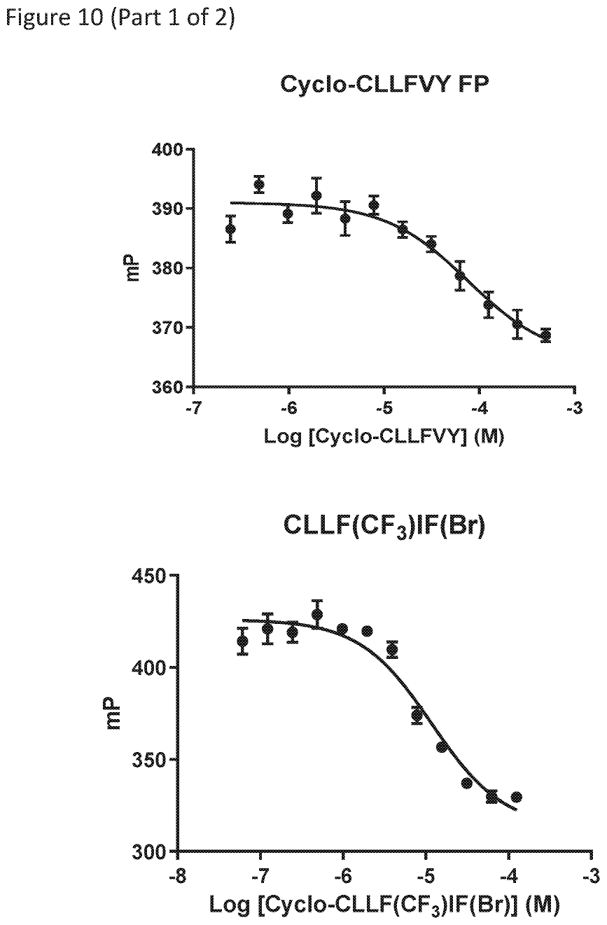

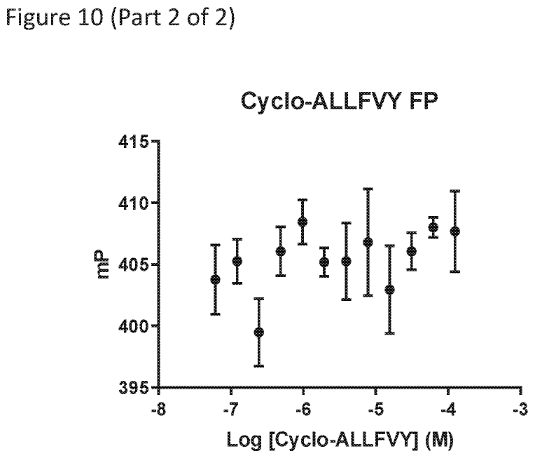

The ability of CK-n-leu-F(CF₃)IF(Br) to disrupt the HIF-1α-HIF-1β PAS-B-PAS-B PPI was then assessed via an ELISA assay. CK-n-leu-F(CF₃)IF(Br) was seen to inhibit dimer formation with an IC50 of 27.7±14 μM—FIG. 9.

### FP

We next sought to show that inhibition of the interaction between the HIF-1α and HIF-1β PAS-B domains by CK-n-leu-F(CF₃)IF(Br) prevents HIF from binding to a hypoxia response element. An FP assay utilizing a FAM labelled HRE was used and CK-NL-F(CF₃)IF(Br) assessed. However, the peptide was found to be incompatible with this assay as it was found to bind to the FAM-HRE directly, most likely as a result of the positively charged peptide interacting favorably with the negatively charged DNA backbone.

To avoid this the peptide CLLF(CF₃)IF(Br) was also assessed and found to inhibit HIF-DNA binding with an IC50

The parent peptide CLLFVY was found to inhibit DNA binding with an IC50 of 76.5±8.0 μM and the peptide CLLF(CF₃)IF(Br) was found to inhibit FAM-HRE binding with an IC50 of 11.76±1.1 μM demonstrating that the improvements seen during the SAR study translates to increased potency when inhibiting DNA binding.

### Coumarin

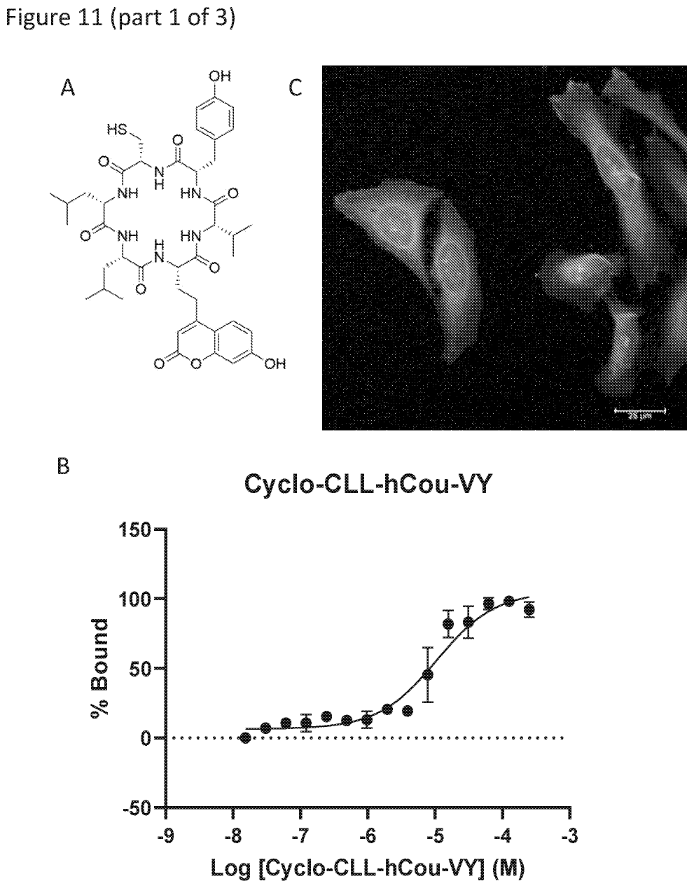

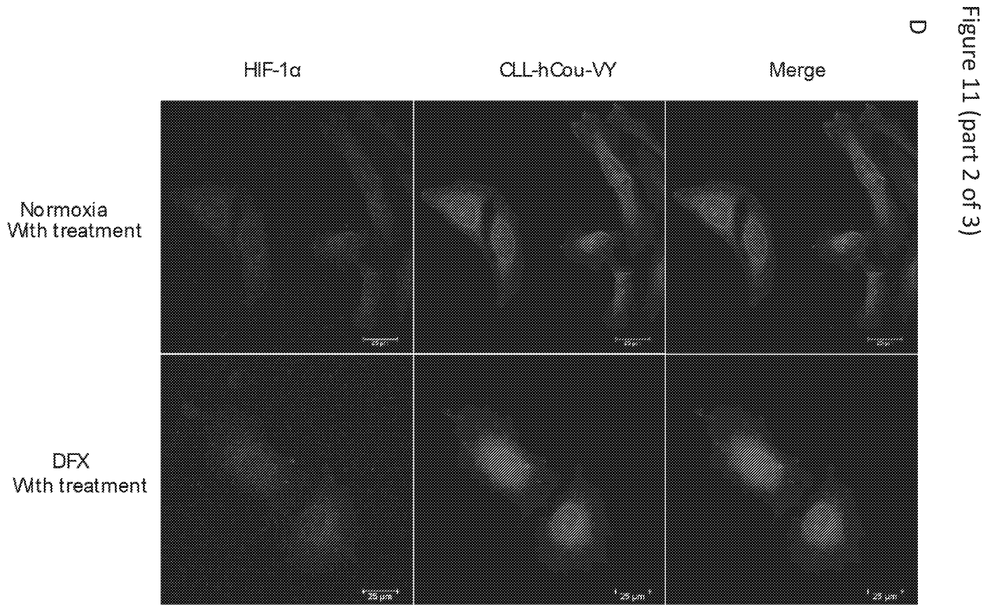

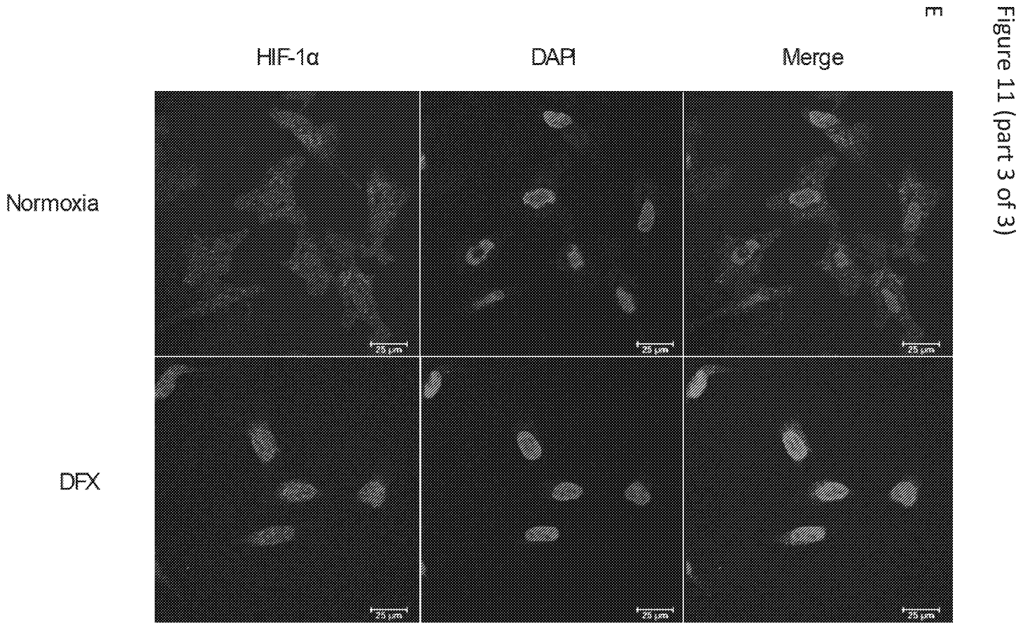

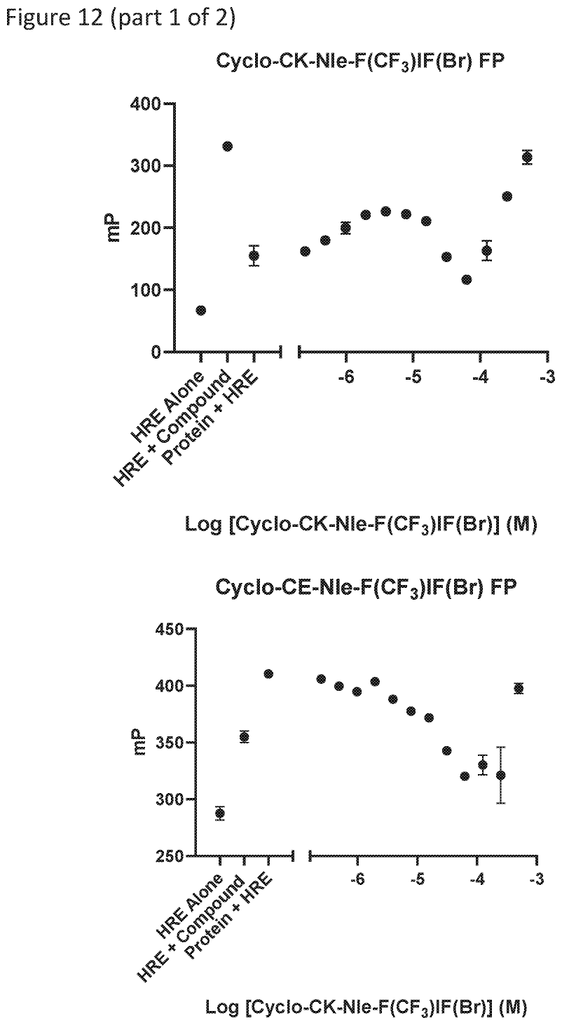

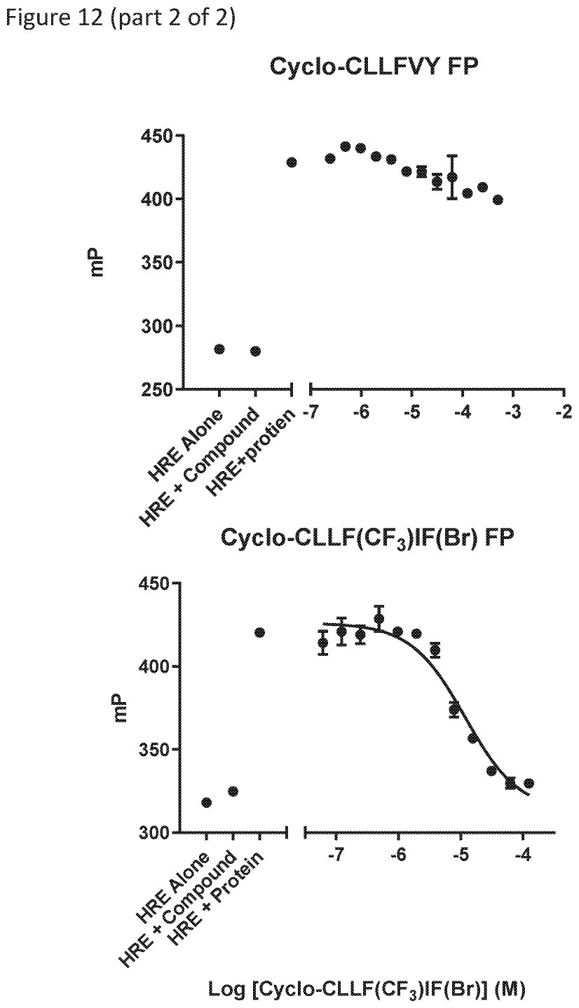

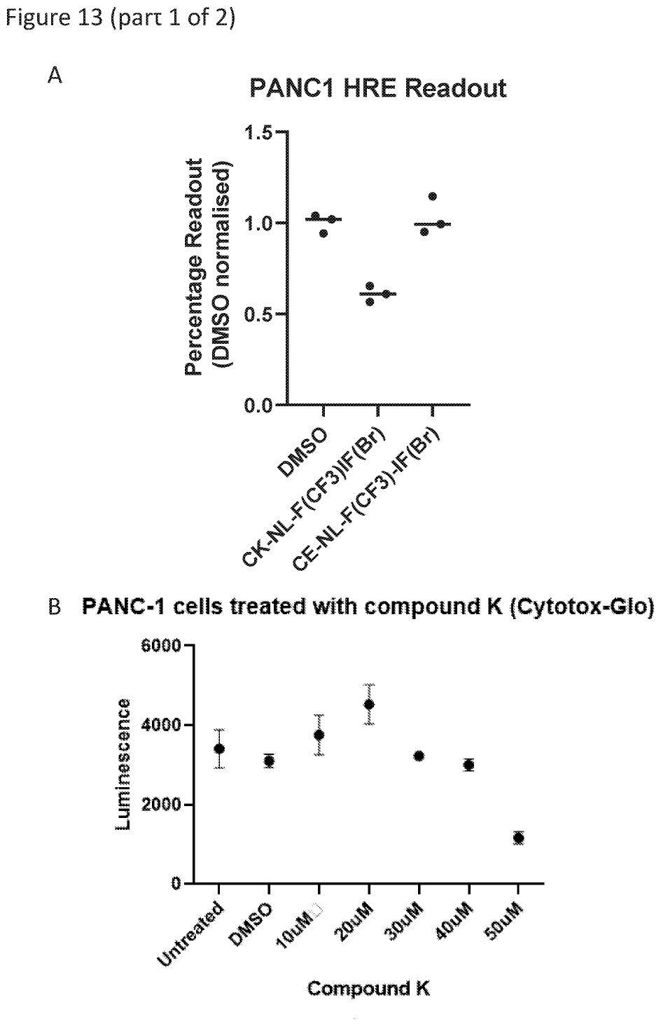

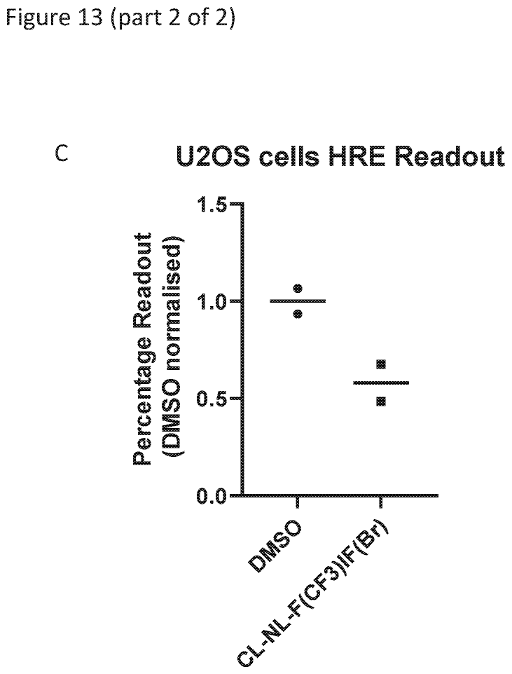

During the investigation of the SAR at the phenylalanine position it was noted that the extended aromatic derivative, Nal, was well tolerated, leading to a 6-fold increase in affinity compared to CLLFVY. The fluorescent molecule coumarin is structurally similar to Nal and has been demonstrated to be well suited for biological applications. We therefore made a peptide with a coumaryl motif incorporated at the phenylalanine position the compound, CLL-Cou-VY, shown in FIG. 11 was seen to be internalized in HeLa cells upon visualization with PMT detection at 405 nm.

HIF-1α immunofluorescence was then used to show that the compound is preventing HIF-1α nuclear localization.

### Methods

### HIF PAS-B MSTs

An aliquot of labelled HIF-1α PAS-B was thawed on ice and diluted to 50 nM with HIF MST assay buffer, before being centrifuged at 12,000 rpm for 15 minutes. The compound was then serially diluted in DMSO to give 10× stocks. These were then diluted 1:5 in HIF MST assay buffer to give 2× stocks, before finally being mixed 1:1 (v/v) with the labelled protein to give a final concertation of 25 nM protein and a final DMSO content of 10%. Samples were loaded into NanoTemper® Monolith NT.115 premium treated capillaries and measurements were carried out using a Monolith NT.115 system at 25° C. using 50% MST power and 50% LED power. Data was analysed by NanoTemper® analysis software. Binding curves were fitted using GraphPad Prism 8 software.

### Enzyme-Linked Immunosorbent Assay

To each well of a clear Pierce nickel coated 96-well plate (Thermo Scientific), purified Hiss-HIF-1α PAS-B protein in HIP buffer was added (25 μL, 0.1 μM), the plate was then incubated for 1 hr at RT with rocking. The wells were then washed 2× with HIF buffer (150 μL) and 1× with PBS Tween (0.05% Tween 20, 150 μL). 2% milk in PBS Tween (150 μL) was then added to each well and incubated for 1 hr at RT with rocking as a blocking step. The wells were then washed 2×PBS Tween (150 μL) and 1× with HIF buffer (150 μL). For HIF-B dimerisation a serial dilution of HIF-1β PAS-B ranging from 250 μM to 11 nM in HIF buffer (100 μL) was then added. For CLLFVY inhibition a serial dilution of CLLFVY ranging from 500 μM to 15 nM in 100% DMSO (10 μL) was added at the same time as HIF-1β PAS-B (10 μM, 90 μL) for a 10% final concentration of DMSO. The plate was then incubated at RT for 1 hr with rocking. The wells were then washed 2× with HIF buffer (150 μL) and 1× with PBS Tween (150 μL), A 1:1000 dilution of mouse anti-flag antibody in 2% milk PBS Tween (50 μL) was then added to each well and the plate incubated for a further hour at RT with rocking. The wells were then washed with 3×PBS Tween (150 μL) with 5 minutes incubation per wash. A 1:6000 dilution of sheep anti mouse linked to horse radish peroxidase in 2% milk PBS Tween (50 μL) was then added and a further incubation of 1 hr at RT with rocking carried out. The plate was then washed as before and 1-Step TMB Ultra Solution (Thermo Scientific) (100 μL) was added to each well and incubated for 15 minutes. H₂SO_{4(aq)} (2 M, 100 μL) was then added to each well and the absorbance at 450 nm determined on a BMG Clariostar plate reader.

### Fluorescent Polarization Assays

HIF-1 three-domain protein was thawed on ice and diluted to 150 nM in HIF FP buffer. Compounds for analysis were serially diluted in DMSO to give 10× stocks. The compounds (10 μl, 100% DMSO) were then added to the protein (80 μL). The samples were then incubated at room temperature for 30 minutes. FAM-HRE (10 μL, 20 nM) was then added and the samples loaded into a 384 well solid black non-binding microplate (Corning, USA). The fluorescence polarization of each well was then measured using gain and focus settings optimized by the used software set to a signal of 400 mP on a control well.

### Example 2—Compound Synthesis and Characterization

### CLLFVY Phenylalanine Derivatives

The linear resin bound sequence resin-LLCYV-NH₂ was synthesised using Wang resin SPPS (0). The resin was then split equally into 10 syringes and swelled in a small amount of DMF. A solution of unnatural amino acid (1 eq.), Oxyma pure (3 eq.) and DIC (3 eq.) was added and the syringes placed on a rocker for 1 hour. The reaction mixtures were then removed via vacuum filtration and the resins washed with DMF×2, DCM×2 and Et₂O×1. Fmoc protected amino acids were then deprotected via the addition of piperidine 20% in DMF and rocking for 30 minutes. The resins were then washed as before. The linear sequences were then cleaved from the resin as described in section 0, and cyclised using HATU, HOAt and DiPEA as described in section 0. The StBu group was then removed (section 0) and the peptide purified via reverse-phase HPLC (section 0), like fractions were combined and concentrated under reduced pressure, then lyophilised to yield the desired cyclic peptides.

Cyclo-CLLY(Me)VY-
SEQ ID NO: 50
Cys leu leu Tyr(me) val tyr

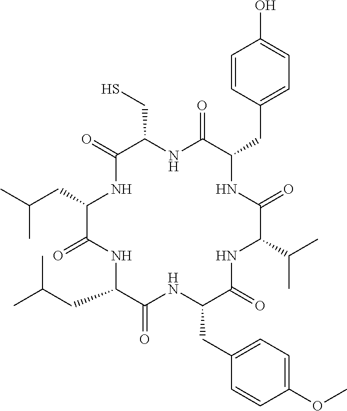 [SMILES: COc1ccc(C[C@@H]2NC(=O)[C@H](CC(C)C)NC(=O)[C@H](CC(C)C)NC(=O)[C@H](CS)NC(=O)[C@H](Cc3ccc(O)cc3)NC(=O)[C@H](C(C)C)NC2=O)cc1]

Cyclo-CLLY(Me)VY was obtained as a white solid yield (3.7 mg, 3.3% overall yield). Analytical HPLC Rₜ=13.419 min (90% purity). m/z (ESI⁺): 769.8 [M+H]⁺ (100%), 791.8 [M+Na]⁺ (25%). HRMS (ESI⁺) found: 769.3944, [M+H]⁺ calculated: 769.3953.

Cyclo-CLLYVY
SEQ ID NO: 51
Cys leu leu tyr val tyr

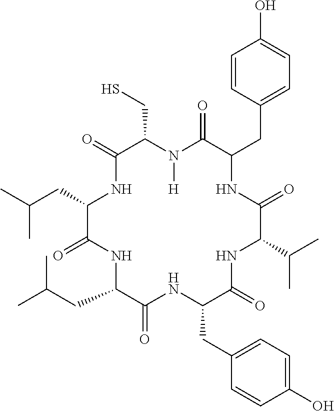 [SMILES: CC(C)C[C@@H]1NC(=O)[C@H](CS)NC(=O)C(Cc2ccc(O)cc2)NC(=O)[C@H](C(C)C)NC(=O)[C@H](Cc2ccc(O)cc2)NC(=O)[C@H](CC(C)C)NC1=O]

Cyclo-CLLYVY was obtained as white solid (25.1 mg, 22.2 Va overall yield). Analytical HPLC Rₜ=11.830 min (99% purity). m/z (ESI⁺): 755.7 [M+H]⁺ (100%). HRMS (ESI⁺) found: 755.3791, [M+H]⁺ calculated: 755.3797.

Cyclo-CLL-PAL-VY
SEQ ID NO: 53
Cys leu leu 4-Pal val tyr

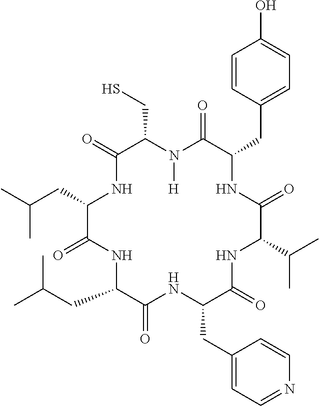 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccncc2)NC(=O)[C@H](CC(C)C)NC1=O]

Cyclo-CLL-PAL-VY was obtained as white solid (16.2 mg, 15.1% overall yield). Analytical HPLC Rₜ=9.721 min (89% purity). m/z (ESI⁺): 740.8 [M+H]⁺ (100%). HRMS (ESI⁺) found: 740.3817, [M+H]⁺ calculated: 740.3800.

Cyclo-CLLF(4-NO₂)VY
SEQ ID NO: 54
Cys leu leu phe(NO2) val tyr

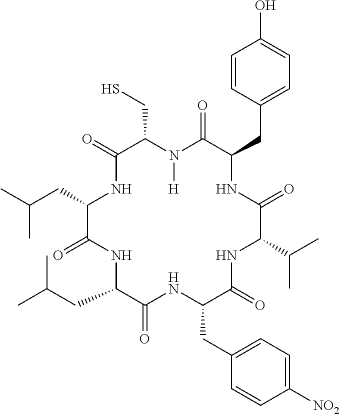 [SMILES: CC(C)C[C@@H]1NC(=O)[C@H](CC(C)C)NC(=O)[C@H](CS)NC(=O)[C@@H](Cc2ccc(O)cc2)NC(=O)[C@H](C(C)C)NC(=O)[C@H](Cc2ccc([N+](=O)[O-])cc2)NC1=O]

Cyclo-CLLF(4-NO₂)VY was obtained as a white solid (11.5 mg, 10.1% overall yield). Analytical HPLC Rₜ=13.580 min (93% purity). m/z (ESI⁺): 784.8 [M+H]⁺ (100%). HRMS (ESI⁺) found: 784.3695, [M+H]⁺ calculated: 784.3698.

Cyclo-CLL-Cha-VY
SEQ ID NO: 2
Cys leu leu Cha val tyr

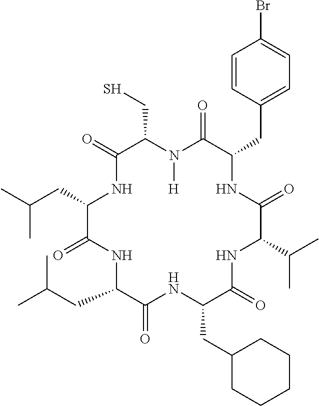 [SMILES: CC(C)C[C@@H]1NC(=O)[C@H](CS)NC(=O)[C@H](Cc2ccc(Br)cc2)NC(=O)[C@H](C(C)C)NC(=O)[C@H](CC2CCCCC2)NC(=O)[C@H](CC(C)C)NC1=O]

Cyclo-CLL-Cha-VY was obtained as a white solid (3.0 mg, 2.9% overall yield). Analytical HPLC Rₜ=15.188 min (99% purity). m/z (ESI⁺): 745.8 [M+H]⁺ (100%), 1491.6 [2 M+H]⁺ (90%). HRMS (ESI⁺) found: 745.4309, [M+H]⁺ calculated: 745.4317.

Cyclo-CLL-Nal-VY
SEQ ID NO: 7
Cys leu leu Nal val tyr

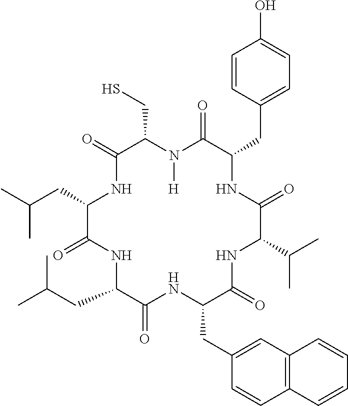 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc3ccccc3c2)NC(=O)[C@H](CC(C)C)NC1=O]

Cyclo-CLL-Nal-VY yield was obtained as a white solid (14.0 mg, 8.9% overall yield). Analytical HPLC Rₜ=15.317 min (92% purity). m/z (ESI⁺): 789.7 [M+H]⁺ (100%), 811.6 [M+Na]⁺ (35%). HRMS (ESI⁺) found: 789.3997, [M+H]⁺ calculated: 789.4004.

Cyclo-CLL-Phg-VY
SEQ ID NO: 52
Cys leu leu PhG val tyr

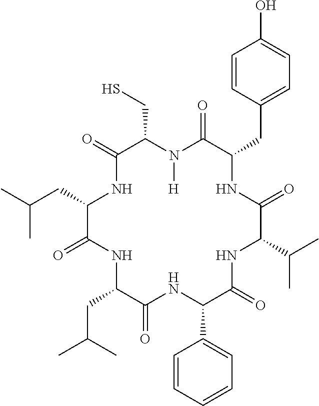 [SMILES: CC(C)C[C@@H]1NC(=O)[C@H](CS)NC(=O)[C@H](Cc2ccc(O)cc2)NC(=O)[C@H](C(C)C)NC(=O)[C@H](c2ccccc2)NC(=O)[C@H](CC(C)C)NC1=O]

Cyclo-CLL-Phg-VY was obtained as a white solid (22.3 mg, 15.4% overall yield). Analytical HPLC Rₜ=12.903 min (87% purity). m/z (ESI⁺): 725.7 [M+H]⁺ (100%), 1450.1 [2 M+H]⁺ (45%). HRMS (ESI⁺) found: 725.3687, [M+H]⁺ calculated: 725.3691.

Cyclo-CLL-hF-VY
SEQ ID NO: 3
Cys leu leu hphe val tyr

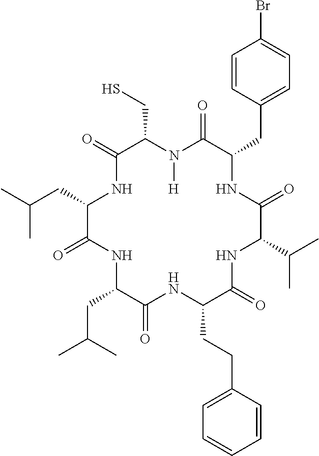 [SMILES: CC(C)C[C@@H]1NC(=O)[C@H](CS)NC(=O)[C@H](Cc2ccc(Br)cc2)NC(=O)[C@H](C(C)C)NC(=O)[C@H](CCc2ccccc2)NC(=O)[C@H](CC(C)C)NC1=O]

Cyclo-CLL-hF-VY was obtained as a white solid (29.5 mg, 19.3% overall yield). Analytical HPLC Rₜ=14.175 min (87% purity). m/z (ESI⁺): 753.7 [M+H]⁺ (100%), 1149.6 [2 M+H]⁺ (45%). HRMS (ESI⁺) found: 753.3991, [M+H]⁺ calculated: 753.4004.

Cyclo-CLL-dF-VY
SEQ ID NO: 6
Cys leu leu dphe val tyr

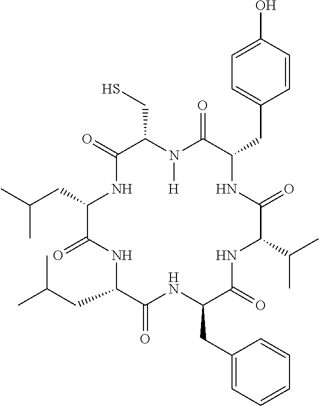 [SMILES: CC(C)C[C@@H]1NC(=O)[C@H](CS)NC(=O)[C@H](Cc2ccc(O)cc2)NC(=O)[C@H](C(C)C)NC(=O)[C@@H](Cc2ccccc2)NC(=O)[C@H](CC(C)C)NC1=O]

Cyclo-CLL-dF-VY was obtained as a white solid (17.9 mg, 14.8% overall yield). Analytical HPLC Rₜ=13.746 min (94% purity). m/z (ESI⁺): 739.8 [M+H]⁺ (100%). HRMS (ESI⁺) found: 739.3846, [M+H]⁺ calculated: 739.3847.

Cyclo-CLLF(F)VY
SEQ ID NO: 55
Cys leu leu phe(4-F) val tyr

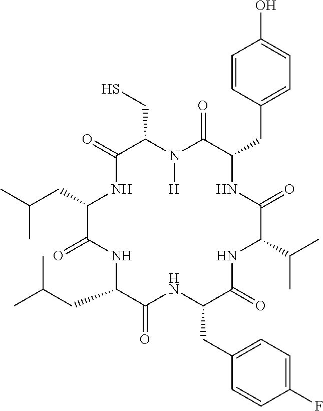 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc(F)cc2)NC(=O)[C@H](CC(C)C)NC1=O]

Cyclo-CLLF(F)VY was obtained as a white solid (11.9 mg, 10.9% overall yield). Analytical HPLC Rₜ=13.905 min (92° % purity). m/z (ESI⁺): 757.8 [M+H]⁺ (100%). HRMS (ESI⁺) found: 757.3751, [M+H]⁺ calculated: 757.3753.

Cyclo-CLLF(Cl)VY
SEQ ID NO: 5
Cys leu leu phe(Cl) val tyr

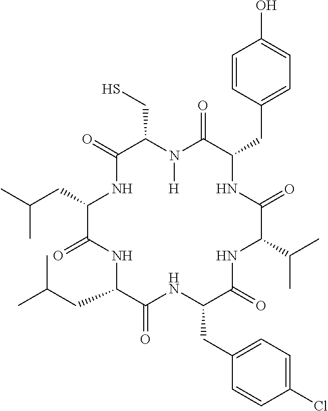 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc(Cl)cc2)NC(=O)[C@H](CC(C)C)NC1=O]

Cyclo-CLLF(Cl)VY was obtained as a white solid (8.5 mg, 7.1 V % overall yield). Analytical HPLC Rₜ=14.519 min (94% purity). m/z (ESI⁺): 773.7 [M+H]⁺ (100%), 795.7 [M+Na]'⁰ (30% a). HRMS (ESI⁺) found: 773.3452, [M+H]⁺ calculated: 773.3458.

Cyclo-CLLF(Br)VY
SEQ ID NO: 8
Cys leu leu phe(Br) val tyr

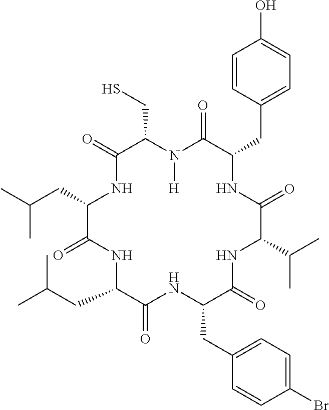 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc(Br)cc2)NC(=O)[C@H](CC(C)C)NC1=O]

Cyclo-CLLF(Br) VY was obtained as a white solid (12.1 mg, 7.4% overall yield). Analytical HPLC Rₜ=14.657 min (90% purity). m/z (ESI⁺): 819.5 [M+H]⁺ (100%). HRMS (ESI⁺) found: 817.2926, [M+H]⁺ calculated: 817.2953.

Cyclo-CLLF(I)VY
SEQ ID NO: 4
Cys leu leu phe(I) val tyr

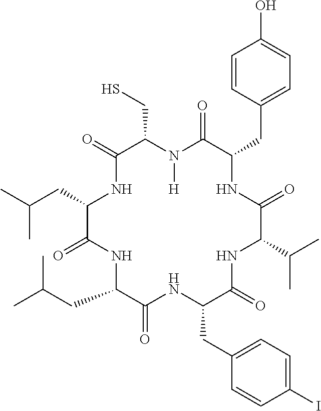 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc(I)cc2)NC(=O)[C@H](CC(C)C)NC1=O]

Cyclo-CLLF(I)VY was obtained as a white solid (15.8 mg, 9.1% overall yield). Analytical HPLC Rₜ=15.144 min (90% purity). m/z (ESI⁺): 865.7 [M+H]⁺ (100%). HRMS (ESI⁺) found: 865.2803, [M+H]⁺ calculated: 865.2814.

Cyclo-CLLF(CF₃)VY
SEQ ID NO: 10
Cys leu leu phe(CF3) val tyr

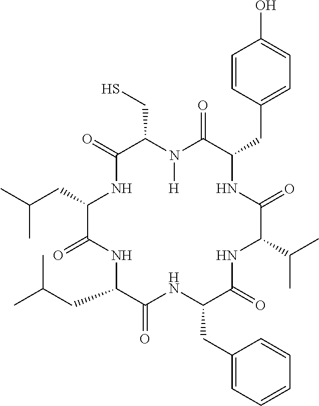 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccccc2)NC(=O)[C@H](CC(C)C)NC1=O]

Cyclo-CLLF(CF₃)VY was obtained as a white solid (29.4 mg, 18.2% overall yield). Analytical HPLC Rₜ=14.840 min (90% purity). m/z (ESI⁺): 807.7 [M+H]⁺ (100%), 829.7 [M+Na]⁺ (60%). HRMS (ESI⁺) found: 807.3720, [M+H]⁺ calculated: 807.3721.

Cyclo-CLLF(tBu)VY
[SEQ ID NO: 56]

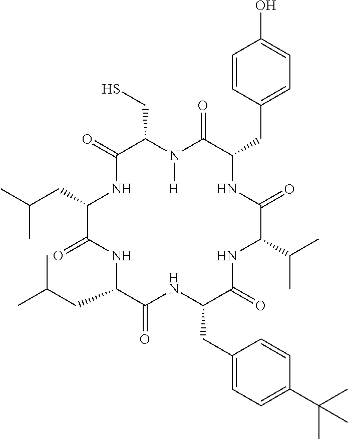 [SMILES: CC(C)C[C@@H]1NC(=O)[C@H](CS)NC(=O)[C@H](Cc2ccc(O)cc2)NC(=O)[C@H](C(C)C)NC(=O)[C@H](Cc2ccc(C(C)(C)C)cc2)NC(=O)[C@H](CC(C)C)NC1=O]

Cyclo-CLLF(4-tBu)VY was obtained as a white solid (16.6 mg, 10.4% overall yield). Analytical HPLC Rₜ=16.384 min (93% purity). m/z (ESI⁺): 795.87 [M+H]⁺ (100%). HRMS (ESI⁺) found: 795.4449, [M+H]⁺ calculated: 795.4473.

### CLLF(CF₃)VY Tyrosine Derivatives

The linear resin bound sequence resin-LC-NH₂ was synthesised using Wang resin SPPS (section 0). The resin was then split equally into 10 syringes and swelled in a small amount of DMF. Standard SPPS protocols (section 0) were then followed to yield the resin bound linear sequence resin-LCXVF(CF₃)L where X is the unnatural amino acid. The linear sequences were then cleaved from the resin as described in section 0, and cyclised using HATU, HOAt and DIPEA as described in section 0. The StBu group was then removed (section 0) and the peptide purified via reverse-phase HPLC (section 0), like fractions were combined and concentrated under reduced pressure, then lyophilised to yield the desired cyclic peptides.

CLLF(CF₃)VY(Me)
SEQ ID NO: 18
Cys leu leu phe(CF3) val Tyr(me)

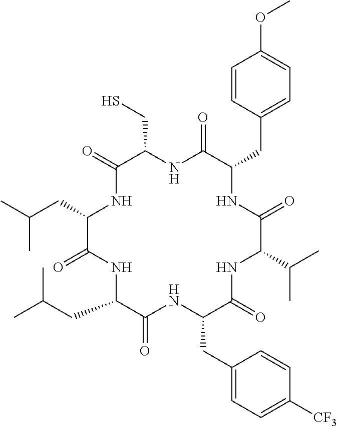 [SMILES: COc1ccc(C[C@@H]2NC(=O)[C@H](C(C)C)NC(=O)[C@H](Cc3ccc(C(F)(F)F)cc3)NC(=O)[C@H](CC(C)C)NC(=O)[C@H](CC(C)C)NC(=O)[C@H](CS)NC2=O)cc1]

Cyclo-CLLF(CF₃)VY(Me) was obtained as a white solid (9.2 mg, 5.6% overall yield). Analytical HPLC Rₜ=16.557 min (99% purity). m/z (ESI⁺): 821.7 [M+H]⁺ (100%). HRMS (ESI⁺) found: 821.3866, [M+H]⁺ calculated: 821.3878.

CLLF(CF₃)VF
SEQ ID NO: 17
Cys leu leu phe(CF3) val phe

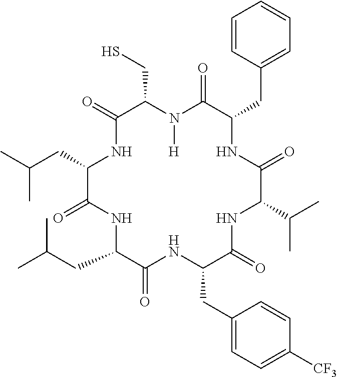 [SMILES: CC(C)C[C@@H]1NC(=O)[C@H](CS)NC(=O)[C@H](Cc2ccccc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF was obtained as a white solid (62.7 mg, 39.7% overall yield). Analytical HPLC Rₜ=16.769 min (95% purity). m/z (ESI⁺): 791.6 [M+H]⁺ (100%), 813.6 [M+Na]⁺ (20%). HRMS (ESI⁺) found: 791.3779, [M+H]⁺ calculated: 791.3772.

CLLF(CF₃)V-Pal
SEQ ID NO: 11
Cys leu leu phe(CF3) val Pal

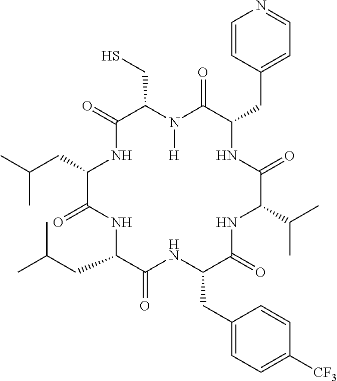 [SMILES: CC(C)C[C@@H]1NC(=O)[C@H](CS)NC(=O)[C@H](Cc2ccncc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-4-Pal was obtained as a white solid (24.7 mg, 15.6% overall yield). Analytical HPLC Rₜ=12.770 min (97% purity), m/z (ESI⁺): 792.6 [M+H]⁺ (100%). HRMS (ESI⁺) found: 792.3716, [M+H]⁺ calculated: 792.3725.

CLLF(CF₃)VF(NO₂)
SEQ ID NO: 21
Cys leu leu phe(CF3) val phe(NO2)

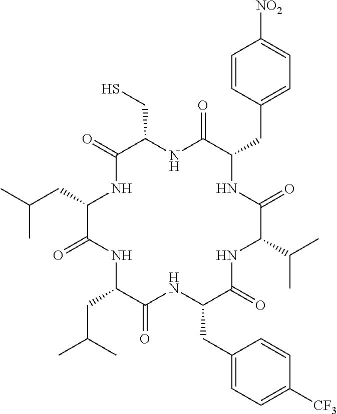 [SMILES: CC(C)C[C@@H]1NC(=O)[C@H](CS)NC(=O)[C@H](Cc2ccc([N+](=O)[O-])cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(NO₂) was obtained as a white solid (20.0 mg, 12.0%). Analytical HPLC Rₜ=12.877 min (99% purity). m/z (ESI⁺): 836.6 [M+H]⁺ (100%). HRMS (ESI⁺) found: 836.3616, [M+H]⁺ calculated: 836.3623.

CLLF(CF₃)V-Cha
SEQ ID NO: 12
Cys leu leu phe(CF3) val Cha

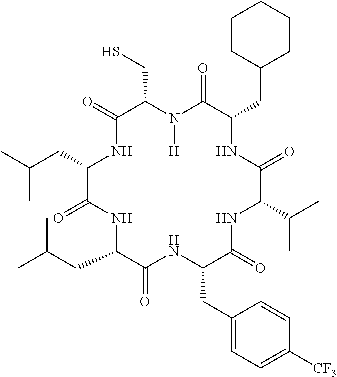 [SMILES: CC(C)C[C@@H]1NC(=O)[C@H](CS)NC(=O)[C@H](CC2CCCCC2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-Cha was obtained as a white solid (16.2 mg, 10.2% overall yield). Analytical HPLC Rₜ=18.111 min (97% purity). m/z (ESI⁺): 797.7 [M+H]⁺ (100%). HRMS (ESI⁺) found: 797.4242, [M+H]⁺ calculated: 797.4242.

CLLF(CF₃)V-Nal
SEQ ID NO: 22
Cys leu leu phe(CF3) val Nal

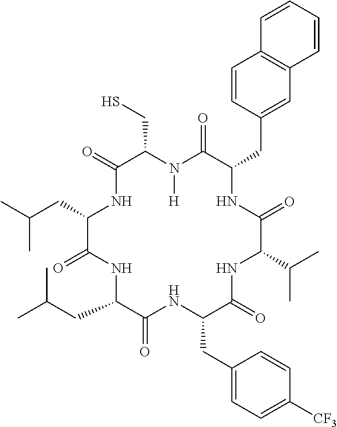 [SMILES: CC(C)C[C@@H]1NC(=O)[C@H](CS)NC(=O)[C@H](Cc2ccc3ccccc3c2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-Nal was obtained as white solid (31.9 mg, 19.0% overall yield). Analytical HPLC Rₜ=17.959 min (99% purity). m/z (ESI⁺): 841.6 [M+H]⁺ (100%). HRMS (ESI⁺) found: 841.3919, [M+H]⁺ calculated: 841.3929.

CLLF(CF₃)V-Phg
SEQ ID NO: 15
Cys leu leu phe(CF3) val Phg

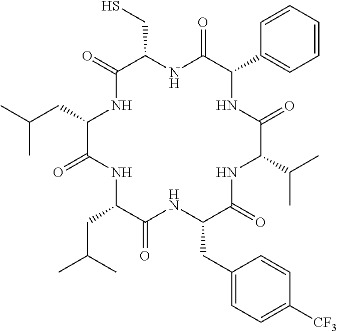 [SMILES: CC(C)C[C@@H]1NC(=O)[C@H](CC(C)C)NC(=O)[C@H](CS)NC(=O)[C@H](c2ccccc2)NC(=O)[C@H](C(C)C)NC(=O)[C@H](Cc2ccc(C(F)(F)F)cc2)NC1=O]

Cyclo-CLLF(CF₃)V-Phg was obtained as a white solid (16.1 mg, 10.4% overall yield). Analytical HPLC Rₜ=16.544 min (92% purity). m/z (ESI⁺): 777.7 [M+H]⁺ (100%). HRMS (ESI⁺) found: 777.3627, [M+H]⁺ calculated: 777.3616.

CLLF(CF₃)V-hF
SEQ ID NO: 20
Cys leu leu phe(CF3) val h-phe

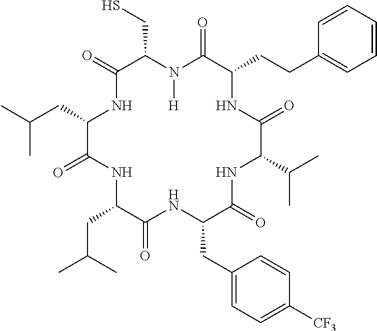 [SMILES: CC(C)C[C@@H]1NC(=O)[C@H](CS)NC(=O)[C@H](CCc2ccccc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-hF was obtained as a white solid (2.7 mg, 1.7% overall yield). Analytical HPLC Rₜ=17.200 min (90% purity). m/z (ESI⁺): 806.7 [M+H]⁺ (100%). HRMS (ESI⁺) found: 805.3933, [M+H]⁺ calculated: 805.3929.

CLLF(CF₃)V-dY
SEQ ID NO: 14
Cys leu leu phe(CF3) val d-tyr

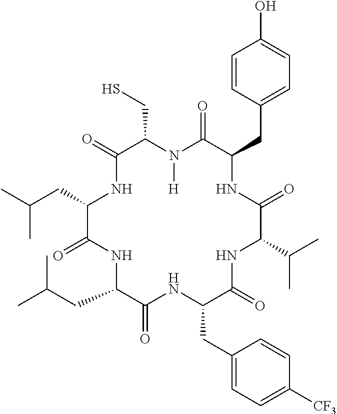 [SMILES: CC(C)C[C@@H]1NC(=O)[C@H](CC(C)C)NC(=O)[C@H](CS)NC(=O)[C@@H](Cc2ccc(O)cc2)NC(=O)[C@H](C(C)C)NC(=O)[C@H](Cc2ccc(C(F)(F)F)cc2)NC1=O]

Cyclo-CLLF(CF₃)V-dY was obtained as a white solid (12.1 mg, 7.5% overall yield). Analytical HPLC Rₜ=14.683 min (97% purity). m/z (ESI⁺): 807.7 [M+H]⁺ (100%). HRMS (ESI⁺) found: 807.3720, [M+H]⁺ calculated: 807.3721.

CLLF(CF₃)VF(F)
SEQ ID NO: 23
Cys leu leu phe(CF3) val phe(4-F)

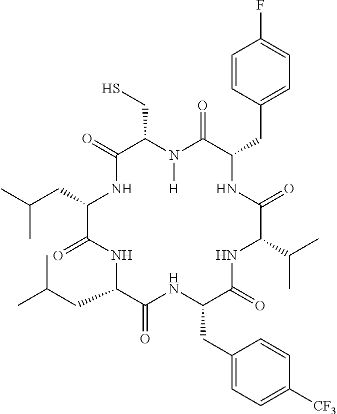 [SMILES: CC(C)C[C@@H]1NC(=O)[C@H](CS)NC(=O)[C@H](Cc2ccc(F)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(F) was obtained as a white solid (28.4 mg, 17.6% overall yield). Analytical HPLC Rₜ=16.665 min (95% purity). m/z (ESI⁺): 809.7 [M+H]⁺ (100%). HRMS (ESI⁺) found: 809.3672, [M+H]⁺ calculated: 809.3678.

CLLF(CF₃)VF(Cl)
SEQ ID NO: 24
Cys leu leu phe(CF3) val phe(4-Cl)

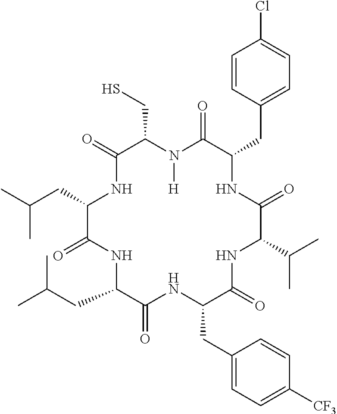 [SMILES: CC(C)C[C@@H]1NC(=O)[C@H](CS)NC(=O)[C@H](Cc2ccc(Cl)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(C) was obtained as a white solid (28.4 mg, 17.2% overall yield). Analytical HPLC Rₜ=17.376 min (99% purity), m/z (ESI⁺): 825.6 [M+H]⁺ (100%). HRMS (ESI⁺) found: 825.3382, [M+H]⁺ calculated: 825.3382.

CLLF(CF₃)VF(Br)
SEQ ID NO: 25
Cys leu leu phe(CF3) val phe(4-Br)

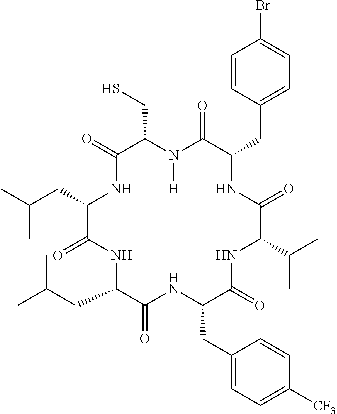 [SMILES: CC(C)C[C@@H]1NC(=O)[C@H](CS)NC(=O)[C@H](Cc2ccc(Br)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(Br) was obtained as a white solid (8.3 mg, 4.8% overall yield). Analytical HPLC Rₜ=17.580 min (99% purity). m/z (ESI⁺): 871.6 [M+H]⁺ (100%). HRMS (ESI⁺) found: 869.2877, [M+H]⁺ calculated: 869.2881.

CLLF(CF₃)VF(I)
SEQ ID NO: 19
Cys leu leu phe(CF3) val phe(4-I)

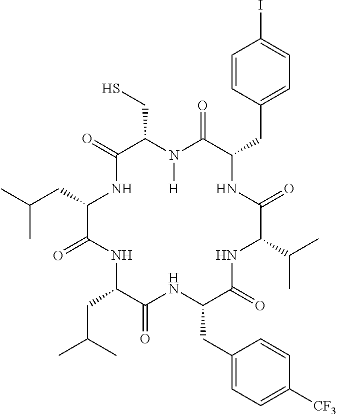 [SMILES: CC(C)C[C@@H]1NC(=O)[C@H](CS)NC(=O)[C@H](Cc2ccc(I)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(I) was obtained as a white solid (19.8 mg, 10.8% overall yield). Analytical HPLC Rₜ=17.893 min (91% purity). m/z (ESI⁺): 917.7 [M+H]⁺ (100%). HRMS (ESI⁺) found: 917.2728, [M+H]⁺ calculated: 917.2739.

CLLF(CF₃)VF(CF₃)
SEQ ID NO: 16
Cys leu leu phe(CF3) val phe(4-CF3)

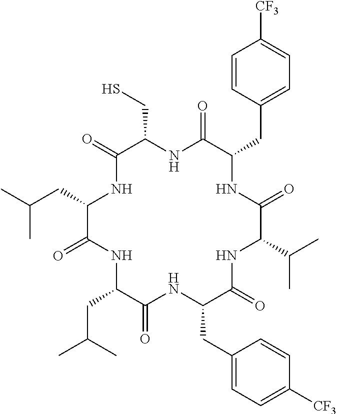 [SMILES: CC(C)C[C@@H]1NC(=O)[C@H](CS)NC(=O)[C@H](Cc2ccc(C(F)(F)F)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)VF(CF₃) was obtained as a white solid (31.6 mg, 18.4%). Analytical HPLC Rₜ=16.284 min (99% purity). m/z (ESI⁺): 859.7 [M+H]⁺. HRMS (ESI⁺) found: 859.3640, [M+H]⁺ calculated: 859.3646.

CLLF(CF₃)VF(tBu)
[SEQ ID NO: 57]

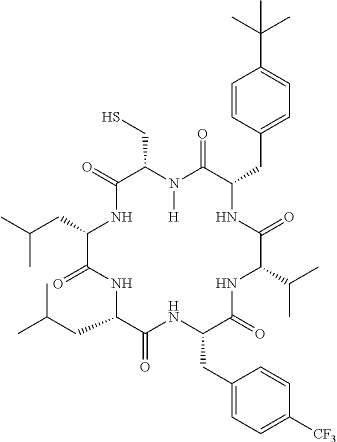 [SMILES: CC(C)C[C@@H]1NC(=O)[C@H](CS)NC(=O)[C@H](Cc2ccc(C(C)(C)C)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-dY was obtained as a white solid (24.1 mg, 14.2%). Analytical HPLC Rₜ=17.333 min 99% purity). m/z (ESI⁺): 847.7 [M+H]⁺ (100%), 869.7 [M+Na]⁺ (60%). HRMS (ESI⁺) found: 847.4391, [M+H]⁺ calculated: 847.4398.

CLLF(CF₃)VF(Ph)
SEQ ID NO: 13
Cys leu leu phe(CF3) val phe(4-Ph)

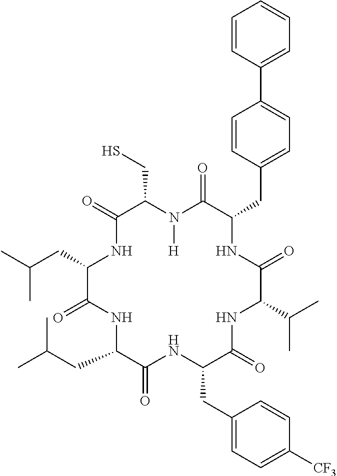 [SMILES: CC(C)C[C@@H]1NC(=O)[C@H](CS)NC(=O)[C@H](Cc2ccc(-c3ccccc3)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)V-dY was obtained as a white solid (29.1 mg, 16.8%). Analytical HPLC Rₜ=18.351 min (98% purity). m/z (ESI⁺): 867.7 [M+H]⁺ (100%), 889.7 [M+Na]⁺ (30%). HRMS (ESI⁺) found: 867.4078, [M+H]⁺ calculated: 867.4085.

### CLLF(CF₃)VF(Br) Valine Derivatives

Fmoc-Leu-Wang resin (0.1 mmol) was added to 8 syringes and the Fmoc group removed via exposure to 20% piperidine in DMF. A solution of amino acid (1 eq.), Oxyma pure (3 eq.) and DIC (3 eq.) were added and the syringes placed on a rocker for 1 hour. The reaction mixtures were then removed via vacuum filtration and the resins washed with DMF×2, DCM×2 and Et₂O×1. Fmoc protected amino acids were then deprotected via the addition of piperidine 20% in DMF and being placed on a rocker for 30 minutes. The resins were then washed as before. These steps were repeated until the desired linear sequence was synthesised. The linear sequences were then cleaved from the resin using a TFA cleavage cocktail (section 0) and cyclised using HATU and DIPEA (section 0) excess DMF was removed under reduced pressure and the crude cyclic peptide used without further purification. The StBu group was then removed (section 0) and the peptide purified via reverse-phase HPLC (section 0), like fractions were combined and concentrated under reduced pressure, then lyophilised to yield the desired cyclic peptides.

CLLF(CF₃)-Aib-F(Br)
SEQ ID NO: 27
Cys leu leu phe(CF3) Aib phe(4-Br)

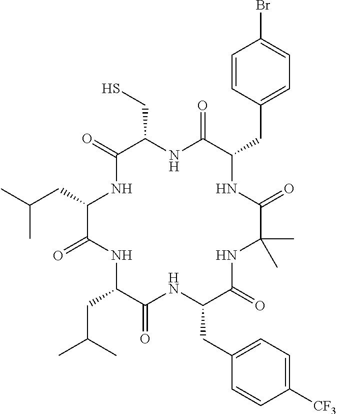 [SMILES: CC(C)C[C@@H]1NC(=O)[C@H](CS)NC(=O)[C@H](Cc2ccc(Br)cc2)NC(=O)C(C)(C)NC(=O)[C@H](Cc2ccc(C(F)(F)F)cc2)NC(=O)[C@H](CC(C)C)NC1=O]

Cyclo-CLLF(CF₃)-Aib-F(Br) was obtained as a white solid (3.3 mg, 3.86%). Analytical HPLC Rₜ=16.835 min (89% purity), m/z (ESI⁺): 857.4 [M+H]⁺ (100%). HRMS (ESI⁺) found: 877.2460, [M+Na]⁺ calculated: 877.2546.

CLLF(CF₃)-Abu-F(Br)
SEQ ID NO: 26
Cys leu leu phe(CF3) Abu phe(4-Br)

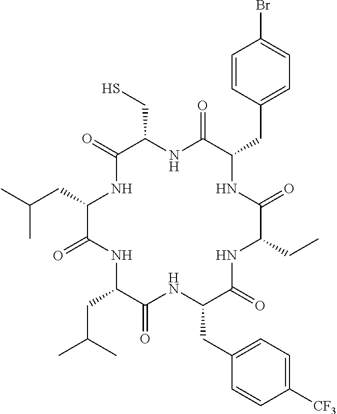 [SMILES: CC[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLLF(CF₃)-Abu-F(Br) was obtained as a white solid (32.9 mg, 38.5%). Analytical HPLC Rₜ=16.609 min (99% purity). m/z (ESI⁺): 857.4 [M+H]⁺ (100%), 879.5 [M+Na]⁺ (40%). HRMS (ESI⁺) found: 877.2537, [M+H]⁺ calculated: 877.2546.

CLLF(CF₃)LF(Br)
SEQ ID NO: 30
Cys leu leu phe(CF3) leu phe(4-Br)

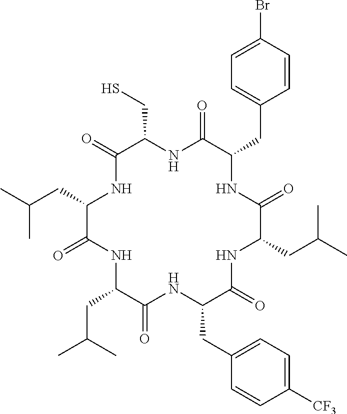 [SMILES: CC(C)C[C@@H]1NC(=O)[C@H](CS)NC(=O)[C@H](Cc2ccc(Br)cc2)NC(=O)[C@H](CC(C)C)NC(=O)[C@H](Cc2ccc(C(F)(F)F)cc2)NC(=O)[C@H](CC(C)C)NC1=O]

Cyclo-CLLF(CF₃)LF(Br) was obtained as a white solid (25.9 mg, 29.4%). Analytical HPLC Rₜ=17.873 min (99% purity). m/z (ESI⁺): 885.5 [M+H]⁺ (100%). HRMS (ESI⁺) found: 905.2863, [M+Na]⁺ calculated: 905.2859.

CLLF(CF₃)-NL-F(Br)
SEQ ID NO: 28
Cys leu leu phe(CF3) n-leu phe(Br)

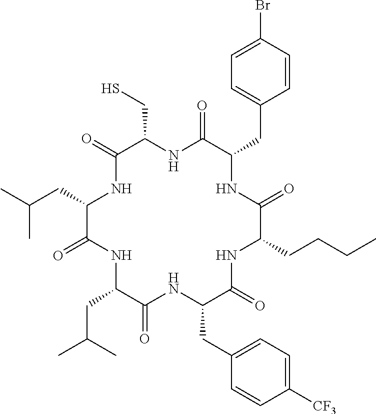 [SMILES: CCCC[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLLF(CF₃)—N-LEU-F(Br) was obtained as a white solid (15.2 mg, 17.2%). Analytical HPLC Rₜ=18.107 min (95% purity). m/z (ESI⁺): 885.5 [M+H]⁺ (100%), 905.4 [M+Na]⁺ (60%). HRMS (ESI⁺) found: 883.3034, [M+H]⁺ calculated: 883.3034.

CLLF(CF₃)-NV-F(Br)
[SEQ ID NO: 58]

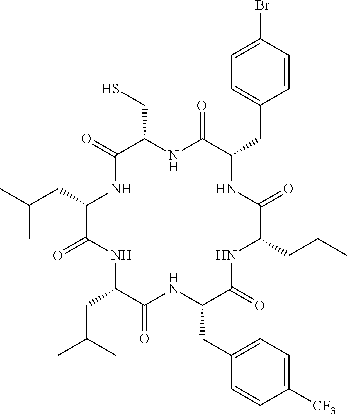 [SMILES: CCC[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLLF(CF₃)—NV—F(Br) was obtained as a white solid (23.8 mg, 27.4%). Analytical HPLC Rₜ=17.340 min (98% purity). m/z (ESI⁺): 893.5 [M+Na]⁺ (100%), 869.5 [M+H]⁺ (90%). HRMS (ESI⁺) found: 869.2873, [M+H]⁺ calculated: 869.2877.

CLLF(CF₃)-hL-F(Br)
[SEQ ID NO: 59]

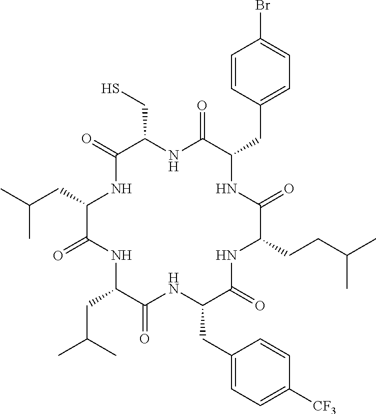 [SMILES: CC(C)CC[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLLF(CF₃)-hL-F(Br) was obtained as a white solid (33.3 mg, 18.6%). Analytical HPLC Rₜ=18.577 min (99% purity). m/z (ESI⁺): 899.7 [M+H]⁺ (100%). HRMS (ESI⁺) found: 897.3166, [M+H]⁺ calculated: 897.3190

CLLF(CF₃)IF(Br)
SEQ ID NO: 31
Cys leu leu phe(CF3) ile phe(4-Br)

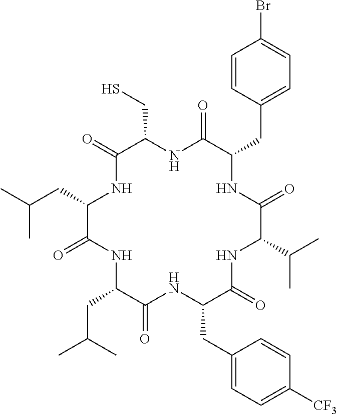 [SMILES: CC(C)C[C@@H]1NC(=O)[C@H](CS)NC(=O)[C@H](Cc2ccc(Br)cc2)NC(=O)[C@H](C(C)C)NC(=O)[C@H](Cc2ccc(C(F)(F)F)cc2)NC(=O)[C@H](CC(C)C)NC1=O]

Cyclo-CLLF(CF₃)IF(Br) was obtained as a white solid (14.2 mg, 8.0%). Analytical HPLC Rₜ=18.149 min (99% purity). m/z (ESI⁺): 885.6 [M+H]⁺ (100%), 907.6 [M+Na]⁺ (40%). HRMS (ESI⁴) found: 883.3020, [M+H]⁺ calculated: 883.3034.

CLLF(CF₃)-Prop-F(Br)
SEQ ID NO: 29
Cys leu leu phe(CF3) Prop phe (4-Br)

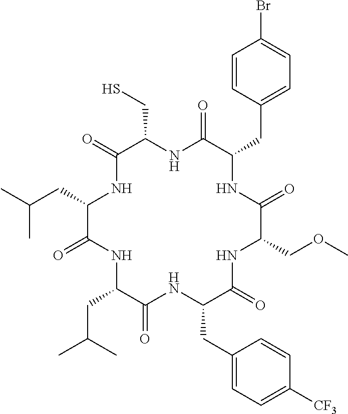 [SMILES: COC[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLLF(CF₃)IF(Br) was obtained as a white solid (14.2 mg, 8.0%). Analytical HPLC Rₜ=16.822 min (98% purity). m/z (ESI⁺873.5 [M+H]⁺ (100%), 893.4 [M+Na]⁺ (99%). HRMS (ESI⁺) found: 871.2660, [M+H]⁺ calculated: 871.2670.

### CLLF(CF₃)IF(Br) Leucine Derivatives

CLVF(CF₃)IF(Br)
SEQ ID NO: 32
Cys leu val phe(CF3) ile phe(4-Br)

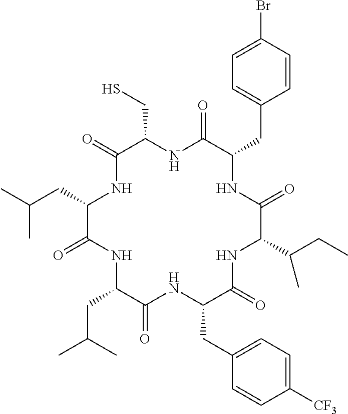 [SMILES: CCC(C)[C@@H]1NC(=O)[C@H](Cc2ccc(C(F)(F)F)cc2)NC(=O)[C@H](CC(C)C)NC(=O)[C@H](CC(C)C)NC(=O)[C@H](CS)NC(=O)[C@H](Cc2ccc(Br)cc2)NC1=O]

Cyclo-CLVF(CF₃)IF(Br) was obtained as a white solid (0.4 mg, 0.5%). Analytical HPLC Rₜ=18.116 min (97% purity). m/z (ESI⁺): 871.6 [M+H]⁺ (100%), 890.6 [M+Na]⁺ (45%). HRMS (ESI⁺) found: 869.2850, [M+H]⁺ calculated: 869.2877.

CL-Prop-F(CF₃)IF(Br)
SEQ ID NO: 36
Cys leu Prop phe(CF3) ile phe(4-Br)

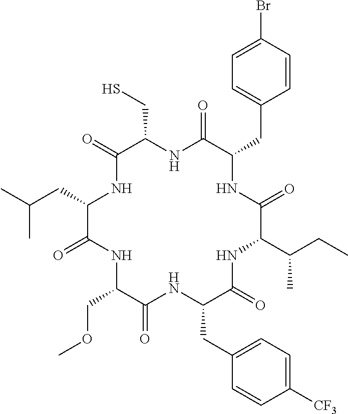 [SMILES: CC[C@H](C)[C@@H]1NC(=O)[C@H](Cc2ccc(C(F)(F)F)cc2)NC(=O)[C@H](COC)NC(=O)[C@H](CC(C)C)NC(=O)[C@H](CS)NC(=O)[C@H](Cc2ccc(Br)cc2)NC1=O]

Cyclo-CL-Prop-F(CF₃)IF(Br) was obtained as white solid (6.1 mg, 7.0%). Analytical HPLC Rₜ=17.001 min (95% purity). m/z (ESI⁺): 873.5 [M+H]⁺ (100%). HRMS (ESI⁺) found: 871.2670, [M+H]⁺ calculated: 871.2670.

CL-NV-F(CF₃)IF(Br)
SEQ ID NO: 35
Cys leu n-val phe(CF3) ile phe(4-Br)

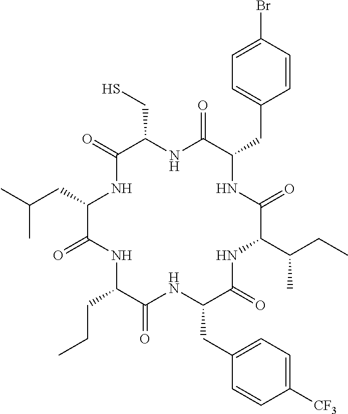 [SMILES: CCC[C@@H]1NC(=O)[C@H](CC(C)C)NC(=O)[C@H](CS)NC(=O)[C@H](Cc2ccc(Br)cc2)NC(=O)[C@H]([C@@H](C)CC)NC(=O)[C@H](Cc2ccc(C(F)(F)F)cc2)NC1=O]

Cyclo-CL-NV—F(CF₃)IF(Br) was obtained as a white solid (6.7 mg, 7.7%). Analytical HPLC Rₜ=17.729 min (99% purity). m/z (ESI⁺): 871.6 [M+H]⁺ (100%). HRMS (ESI⁺) found: 869.2853, [M+H]⁺ calculated: 8692877.

CL-hL-F(CF₃)IF(Br)
SEQ ID NO: 34
Cys leu h-leu phe(CF3) ile phe(4-Br)

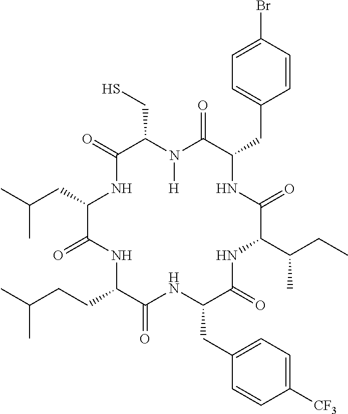 [SMILES: CC[C@H](C)[C@@H]1NC(=O)[C@H](Cc2ccc(C(F)(F)F)cc2)NC(=O)[C@H](CCC(C)C)NC(=O)[C@H](CC(C)C)NC(=O)[C@H](CS)NC(=O)[C@H](Cc2ccc(Br)cc2)NC1=O]

Cyclo-CL-hL-F(CF₃)IF(Br) was obtained as a white solid (16.2 mg, 18.1%). Analytical HPLC Rₜ=18.838 min (99% purity). m/z (ESI⁺): 899.7 [M+H]⁺ (100%). HRMS (ESI⁺) found: 897.3166, [M+H]⁺ calculated: 897.3190.

CL-Aib-F(CF₃)IF(Br)
SEQ ID NO: 33
Cys leu Aib phe(CF3) ile phe(4-Br)

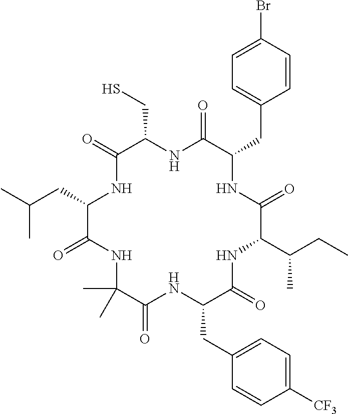 [SMILES: CC[C@H](C)[C@@H]1NC(=O)[C@H](Cc2ccc(C(F)(F)F)cc2)NC(=O)C(C)(C)NC(=O)[C@H](CC(C)C)NC(=O)[C@H](CS)NC(=O)[C@H](Cc2ccc(Br)cc2)NC1=O]

Cyclo-CL-Aib-F(CF₃)IF(Br) was obtained as a white solid (24.7 mg, 28.9%). Analytical HPLC Rₜ=17.737 min (96% purity). m/z (ESI⁺) 857.6 [M+H]⁺ (100%). HRMS (ESI⁺) found: 855.2721, [M+H]⁺ calculated: 855.2721.

CLI F(CF₃)IF(Br)
SEQ ID NO: 37
Cys leu ile phe(CF3) ile phe(4-Br)

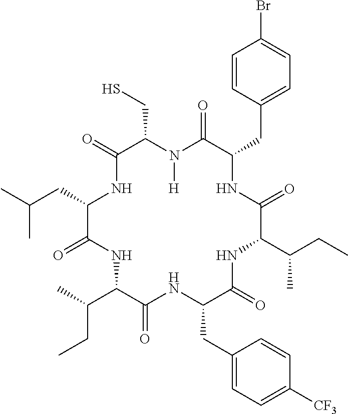 [SMILES: CC[C@H](C)[C@@H]1NC(=O)[C@H](Cc2ccc(C(F)(F)F)cc2)NC(=O)[C@H]([C@@H](C)CC)NC(=O)[C@H](CC(C)C)NC(=O)[C@H](CS)NC(=O)[C@H](Cc2ccc(Br)cc2)NC1=O]

Cyclo-CLIF(CF)IF(Br) was obtained as a white solid (4.5 mg, 5.1%). Analytical HPLC Rₜ=18.566 min (96% purity). m/z (ESI⁺): 885.6 [M+H]⁺ (100%). HRMS (ESI⁺) found: 883.3020, [M+H]⁺ calculated: 883.3034.

CL-NL-F(CF₃)IF(Br)
SEQ ID NO: 38
Cys leu n-leu phe(CF3) ile phe(4-Br)

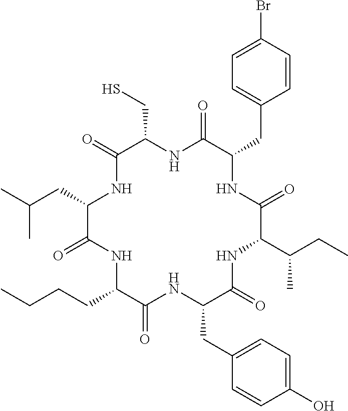 [SMILES: CCCC[C@@H]1NC(=O)[C@H](CC(C)C)NC(=O)[C@H](CS)NC(=O)[C@H](Cc2ccc(Br)cc2)NC(=O)[C@H]([C@@H](C)CC)NC(=O)[C@H](Cc2ccc(O)cc2)NC1=O]

Cyclo-CL-NL-F(CF₃)IF(Br) was obtained as a white solid (12.5 mg, 14.2%). Analytical HPLC Rₜ=18.310 min (99% purity). m/z (ESI⁺): 885.6 [M+H]⁺ (100%). HRMS (ESI⁺) found: 883.3021, [M+H]⁺ calculated: 883.3034.

### Polar Derivatives

CK-NL-F(CF₃)IF(Br)
SEQ ID NO: 40
Cys lys n-leu phe(CF3) ile phe(4-Br)

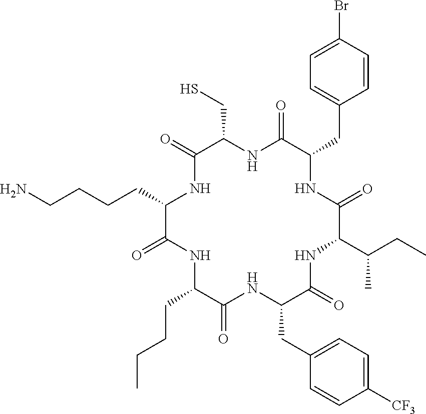 [SMILES: CCCC[C@@H]1NC(=O)[C@H](CCCCN)NC(=O)[C@H](CS)NC(=O)[C@H](Cc2ccc(Br)cc2)NC(=O)[C@H]([C@@H](C)CC)NC(=O)[C@H](Cc2ccc(C(F)(F)F)cc2)NC1=O]

2-chlorotrityl chloride resin was loaded with Fmoc-Lys(Boc) as detailed in section 0 and the resin loading determined (section 0). The linear sequence K(Boc)C(trt)F(Br)IF(CF₃)-NL was then synthesised via Fmoc SPPS (section 0). The linear sequence was then cleaved from the resin using HFIP, leaving the protecting groups intact (section 0). The protected linear was then cyclised with HATU (section 0) to yield the protected cyclic peptide that was immediately globally deprotected with a TFA cleavage cocktail (section 0). The crude product was then dissolved in DMF before being purified via reverse-phase HPLC (section 0) to yield the desired cyclic peptide sequence cyclo-CK-NL-F(CF₃)IF(Br) as a white powder (51.2 mg, 28.5%). Analytical HPLC Rₜ=14.309 min (90.0% purity). m/z (ESI⁺): 900.5 [M+H]⁺ (100%). HRMS (ESI⁺) found: 898.3061, [M+H]⁺ calculated: 898.3143.

CE-NL-F(CF₃)IF(Br)
SEQ ID NO: 41
Cys glu n-leu phe(CF3) ile phe(4-Br)

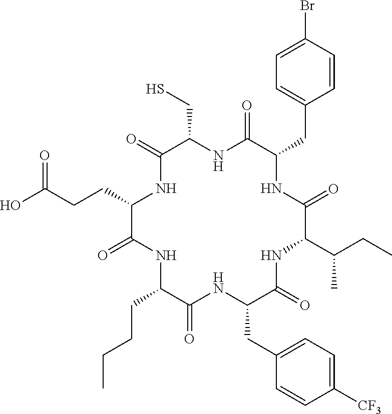 [SMILES: CCCC[C@@H]1NC(=O)[C@H](CCC(=O)O)NC(=O)[C@H](CS)NC(=O)[C@H](Cc2ccc(Br)cc2)NC(=O)[C@H]([C@@H](C)CC)NC(=O)[C@H](Cc2ccc(C(F)(F)F)cc2)NC1=O]

2-chlorotrityl chloride resin was loaded with Fmoc-Glu(OtBu) as detailed in section 0 and the resin loading determined (section 0). The linear sequence E(OtBu)C(trt)F(Br)IF(CF₃)-NL was then synthesised via Fmoc SPPS (section 0). The linear sequence was then cleaved from the resin using HFIP, leaving the protecting groups intact (section 0). The protected linear was then cyclised with HATU (section 0) to yield the protected cyclic peptide that was immediately globally deprotected with a TFA cleavage cocktail (section 0). The crude product was then dissolved in DMF before being purified via reverse-phase HPLC (section 0) to yield the desired cyclic peptide sequence cyclo-CE-NL-F(CF₃)IF(Br) as a white powder (5.7 mg, 3.2%). Analytical HPLC Rₜ=15.749 min (97% purity). m/z (ESI⁺): 901.4 [M+H]⁺ (100%). HRMS (ESI⁺) found: 899.2605, [M+H]⁺ calculated: 899.2619.

### Chapter 1 Coumarin Synthesis

### 1-Benzyl 7-ethyl (S)-2-(((benzyloxy)carbonyl)amino)-5-oxoheptanedioate

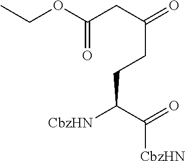 [SMILES: CCOC(=O)CC(=O)CC[C@H](NC(=O)OCc1ccccc1)C(=O)NC(=O)OCc1ccccc1]

Cbz-Glu-OBn (2.00 g, 5.39 mmol, 1 eq.) was dissolved in THF (25 mL) and CDI (0.96 g, 5.92 mmol, 1.1 eq.) was added. The mixture was then stirred at RT for 2 hours under argon. Ethyl magnesium malonate (0.95 g, 2.96 mmol, 0.55 eq.) was then added and the mixture stirred under argon for a further 16 hours. The product was extracted with diethyl ether (3×50 mL) and washed with NaHCO₃ (3×50 mL), water (3×50 mL) and brine (3×50 mL). The organic layer was then dried with anhydrous MgSO₄ and concentrated under reduced pressure to yield a clear oil. The crude product was purified by silica column chromatography with an eluent of 1:1 PE:EtOAc to yield the β-ketoester (2.24 g, 94.1%) as a white solid. m/z (ESI⁺): 464.4 [M+Na]⁺ (100%). ¹H NMR (400 MHz, CDCl₃) δ ppm 1.28 (t, J=7.09 Hz, —OCH₂CH₃, 3H) 1.90-2.28 (m, NHCHCH₂CH₂, 2H) 2.46-2.70 (m, NHCHCH₂CH₂, 2H) 3.39 (s, COCH₂COO, 2H) 4.17 (q, 3=7.09 Hz, —OCH₂CH₃, 2H) 4.43 (td, 3=8.01, 5.26 Hz, NHCHCH₂CH₂, 1H) 5.12 (s, PhCH2OCONH, 2H) 5.19 (s, COOCH2Ph, 2H) 5.44 (br d, J=8.07 Hz, NHCHCH2CH2, 1H) 7.28-7.39 (m, ArH, 10H). Spectral data matches literature²²⁷

### (S)-2-Amino-4-(7-hydroxy-2-oxo-2H-chromen-4-yl)butanoic acid (H2N-hCou-OH)

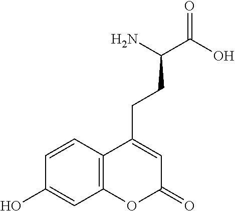 [SMILES: N[C@H](CCc1cc(=O)oc2cc(O)ccc12)C(=O)O]

The β-ketoester (1.0 g, 2.27 mmol, 1 eq.) was added slowly to a solution of resorcinol (1.25 g, 11.35 mmol, 5 eq.) in methanesulfonic acid (5 mL). The solution was stirred at RT for 2 hours before being diluted with ice-cold ether (300 mL). The precipitate was isolated via filtration and then dissolved in water before being filtered again and lyophilised. The resulting residue was purified via RP-chromatography to give the coumaryl amino acid (0.170 g, 28.5%) as an off-white solid. m/z (ESI⁺): 264.2 [M+H]⁺ (100%). ¹H NMR (400 MHz, DMSO-d₆) δ ppm 7.65 (d, J=8.80 Hz, 1H) 6.83 (dd, J=8.68, 2.32 Hz, 1H) 6.75 (d, J=2.45 Hz, 1H) 6.12 (s, 1H) 3.85 (br t, J=5.99 Hz, 1H) 2.80-2.97 (m, 2H) 2.00-2.19 (m, 2H). Spectral data matches literature²²⁷

### (S)-2-((Tert-butoxycarbonyl)amino)-4-(7-((tert-butoxycarbonyl)oxy)-2-oxo-2H-chromen-4-yl)butanoic acid (Boc-hCou(Boc)-OH)

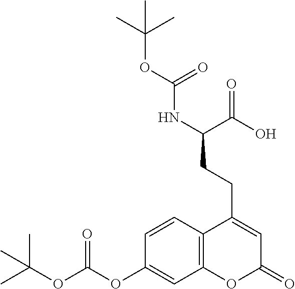 [SMILES: CC(C)(C)OC(=O)N[C@H](CCc1cc(=O)oc2cc(OC(=O)OC(C)(C)C)ccc12)C(=O)O]

The coumaryl amino acid (0.4 g, 1.5 mmol, 1 eq.) was dissolved in 5% aqueous NaHCO₃/dioxane (1:1 v/v) (40 mL) and placed in an ice bath. Boc anhydride (3.27 g, 15 mmol, 10 eq.) was then added and the reaction stirred on ice for 1 hour before being warmed up to RT over 16 hours. The mixture was then acidified to pH 3 with 10% (w/v) citric acid_{(aq)} (60 mL) and the resulting solution extracted with EtOAc (3×75 mL). The extract was then washed with water (3×100 mL) and brine (3×100 mL) before being dried over anhydrous MgSO₄ and concentrated under reduced pressure. The resulting oil was purified via RP-chromatography to yield a mixture of the mono-boc coumaryl amino acid (17.4 mg, 3.2%) and di-boc coumaryl amino acid and (379.4, 54.6%). m/z (ESI⁺): 408.3 [M−tBu+H]⁺ (100%). ¹H NMR (400 MHz, DMSO-d₆) δ ppm 7.92 (d, J=8.80 Hz, 1H), 7.38 (d, J=2.45 Hz, 1H), 7.32 (d, J=8.07 Hz, 1H), 7.26 (dd, J=8.68, 2.32 Hz, 1H), 6.34 (s, 1H), 3.86-4.10 (m, 1H), 2.87 (br t, J=7.58 Hz, 2H), 1.85-2.10 (m, 2H), 1.52 (s, 9H), 1.41 (s, 9H).

### CLL-hCou-VY SEQ ID NO: 39 Cys Leu Leu Cou Val Tyr

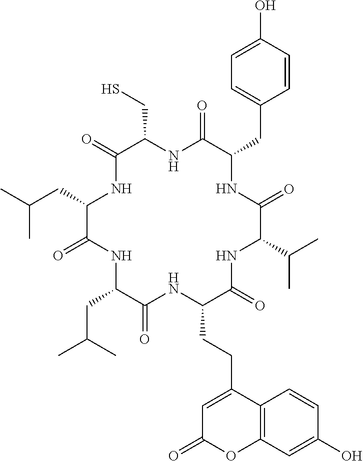 [SMILES: CC(C)C[C@@H]1NC(=O)[C@H](CS)NC(=O)[C@H](Cc2ccc(O)cc2)NC(=O)[C@H](C(C)C)NC(=O)[C@H](CCc2cc(=O)oc3cc(O)ccc23)NC(=O)[C@H](CC(C)C)NC1=O]

Cyclo-CLL-hCou-VY was obtained as a white solid (9.4 mg, 11.2%). Analytical HPLC Rₜ=10.990 min (91% purity). m/z (ESI⁺): 837.5 [M+H]⁺ (100%), 859.5 [M+Na]⁺ (100%). HRMS (ESI⁺) found: 836.3769, [M+H]⁺ calculated: 836.3779.

(ESI⁺) found: 889.3768, [M+H]⁺ calculated: 889.3776.

### Chemical Procedures

### Wang Resin SPPS

Wang resins pre-loaded with Fmoc amino acids were swollen in DMF for 10 minutes with agitation via argon flow. Fmoc deprotection was achieved via the addition of piperidine 20% (v/v) in DMF. Fmoc protected amino acids were then coupled to the resin via the addition of a solution of the amino acid (3 eq.), Oxyma Pure (3 eq.) and DIC (3 eq.) in DMF and leaving the mixture with agitation for 1 hour. The resin was then washed with 3×DMF, 3×DCM and 3×Et₂O and coupling confirmed via Kaiser test. Deprotection of the Fmoc group was again achieved via the addition of piperidine 20% (v/v) in DMF with agitation for 30 minutes. The resin was washed as previously described and deprotection confirmed via Kaiser Test. These steps were repeated until the desired resin bound sequence was achieved.

### Cleavage of Wang Resin

Cleavage of the linear sequence and simultaneous deprotection of side chain protecting groups was achieved using a cleavage cocktail of TFA (4.75 mL), H₂O (0.125 mL) and TIS (0.125 mL) and stirring for 2 hours at RT. The resin was then removed via filtration and the TFA removed under reduced pressure. The peptide was then precipitated with ice cold (−80° C.) diethyl ether. The ether was decanted and the peptide dried under reduced pressure.

### 2-Chlorotrityl Chloride Resin Loading

2-Chlorotrityl chloride resin (1 eq.) was placed in a sinter funnel and DCM (10 mL) added. The resin was then agitated via argon flow for 15 minutes to swell the resin. The DCM was then removed from the funnel and the Fmoc amino acid to be loaded (1 eq.) was dissolved in DCM (10 mL) and added to the resin. DIPEA (3 eq.) was then added and the resin agitated via argon flow for 2 hours. MeOH (1 mL) was then added to the funnel to end cap any unreacted sites and the resin agitated with argon flow for a further 15 minutes. The reaction mixture was then removed under reduced pressure and the resin washed with 3×DCM, 3×DMF and 3×Et₂O. The resin was then dried under reduced pressure.

### Determining Fmoc-AA Resin Loading

Approximately 10 mg of dry resin was accurately weighed into a glass vial and DMF (800 μL) added. The resin was then left to swell for 15 minutes. Piperidine (200 μL) was then added and the resin left for a further 15 minutes. An aliquot of the reaction mixture (100 μL) was then diluted with DMF (900 μL). The resin loading was then determined from the UV absorbance of the dibenzofulvene-piperidine adduct at a wavelength of 301 nm as determined using a NanoDrop ND-1000 Spectrophotometer (NanoDrop Technologies, USA).

### 2-Chlorotrityl Chloride Resin SPPS

Pre-loaded 2-chlorotirityl chloride resins were deprotected with 20% piperidine in DMF. The linear sequence was then synthesised as described for Fmoc-Wang resin synthesis.

### 2-Chlorotrityl Chloride Resin Cleavage

To yield protected linear peptides, linear bound 2-chlorotrityl chloride resin was transferred to a vial and 1,1,1,3,3,3-hexafluroisopropanol (HFIP) (5 mL) and DCM (5 mL) added. The mixture was then stirred at RT for 2 hrs. The resin was then removed via filtration and the solvent removed under reduced pressure. The peptide was then precipitated using ice cold (−80° C.) diethyl ether. The ether was decanted and the peptide dried under reduced pressure.

### Cyclisation via EDC/Oxyma Pure

Linear peptides were dissolved in DMF (1 mL/mg) and EDC (3 eq.) and Oxyma pure (3 eq.) were added. The mixture was then stirred at RT overnight. Excess DMF was removed under reduced pressure and the crude solution purified via reverse phase flash chromatography.

### Cyclisation Via HATU

Linear peptides were dissolved in DMF (1 mL/mg) and HATU (3 eq.) DIPEA (3 eq.) and HOAt (1 eq.) were added. The mixture was then stirred at RT overnight. Excess DMF was removed under reduced pressure and the crude solution purified via reverse phase flash chromatography.

### Cys(StBu) Deprotection

Cys(StBu) containing sequences were deprotected by dissolving the cyclic peptide in DMF (1 mL) and adding DTT (10 eq.) and DIPEA (10 eq.) the mixture was then stirred for 30 minutes at RT. The mixture was then filtered through a 0.22 μm PTFE syringe filter and purified via reverse-phase HPLC.

### Deprotection of Protected Cyclic Peptides

Protected cyclic peptides were dissolved in TFA cleavage cocktail (TFA (4.75 mL), H₂O (0.125 mL) and TIS (0.125 mL)) and stirred at room temperature for 1 hour. Excess TFA was then removed under reduced pressure.

### Kaiser Free Amine Test

After each amino acid coupling step and Fmoc deprotection step, a few beads were transferred into a vial and 10 drops of KCN (0.02 mM) in pyridine and 3 drops of ninhydrin (0.30 M) in ethanol were added. The mixture was then heated to 130° C. for 2 minutes and the presence or absence of blue colour determined if the step was complete.

### Purification via HPLC

Peptides were manually injected as solutions in either DMF or ACN via a Waters Flex inject system into a RP-HPLC system consisting of a Waters 1525 binary pump coupled to a Waters 2998 photodiode array detector. Peptides were then purified using a Waters X Select CSH prep C18 5 μm OBD 19×250 mm column. A binary solvent system consisting of solution A (0.1% TFA/water) and B (0.1% TFA/MeCN) was run at the gradient shown below and elution monitored at 220 nm (peptide backbone). Fractions were collected automatically when the UV trace was in excess of a set threshold. Like fractions were combined and the ACN removed under reduced pressure. The sample was the lyophilised to yield the desired product.

| % A | % B | Time (min) |
|---|---|---|
| 95 | 5 | 0 |
| 10 | 90 | 0-15 |
| 10 | 90 | 15-20 |

### Analytical HPLC

Analytical HPLCs were carried out on an Agilent 1260 Infinity II HPLC system running a binary solvent system consisting of solution A (0.1% TFA/water) and B (0.1% TFA/MeCN). Peptides were injected onto a Poroshell 120 EC-C18 column (2.7 μm particle size, 3.0×100 mm) and the gradient shown below run at a flow rate of 0.625 mL/min. UV absorbance was recorded at both 220 and 280 nm.

| % A | % B | Time (min) |
|---|---|---|
| 95 | 5 | 0 |
| 0 | 100 | 0-20 |
| 0 | 100 | 20-22 |

### Purification via Flash Chromatography

A Biotage Isolera One system equipped with a SNAP Ultra 30 g HP-Sphere C18 25 μm column was used. A binary solvent system consisting of solution A (0.1% TFA/water) and B (0.1% TFA/MeCN) was run at 80% A 20% B for 3CV at 25 mL/min the peptide was then injected directly onto the column as a solution in either DMF or ACN.

A gradient was then run with the solvent steps shown below. Fractions were collected automatically when UV_{220 nm} exceeded 40 mAu. Collected fractions were analysed via mass spectrometry to confirm their identity and the desired fractions were concentrated under reduced pressure and then lyophilised to yield the desired product.

| % A | % B | Number of column volumes |
|---|---|---|
| 80 | 20 | 2 |
| 10 | 90 | 7 |
| 10 | 90 | 2 |

### ESI⁺ MS

Samples were analysed using a Waters (Manchester, UK) TQD mass spectrometer equipped with a triple quadrupole analyser. Samples were introduced to the mass spectrometer via an Acquity H-Class quaternary solvent manager (with TUV detector at 254 nm, sample and column manager). Ultra-performance liquid chromatography was undertaken via a Waters BEH C18 column (50 mm×2.1 mm 1.7 μm).

Gradient 20% acetonitrile (0.2% formic acid) to 100% acetonitrile (0.2% formic acid) in five minutes at a flow rate of 0.6 mL/min. Low-resolution mass spectra were recorded using positive ion electrospray ionisation.

### High Resolution Mass Spectrometry (HRMS)

Samples were analysed using a MaXis (Bruker Daltonics, Bremen, Germany) mass spectrometer equipped with a Time of Flight (TOF) analyser. Samples were introduced to the mass spectrometer via a Dionex Ultimate 3000 autosampler and uHPLC pump. Gradient 20% acetonitrile (0.2% formic acid) to 100% acetonitrile (0.2% formic acid) in five minutes at 0.6 mL/min. Column, Acquity UPLC BEH C18 (Waters) 1.7 micron 50×2.1 mm. High resolution mass spectra were recorded using positive ion electrospray ionisation.

¹H and ¹³C NMR

NMR spectra were recorded on Bruker AVII400 or Bruker AVIIIHD400 FT-NMR spectrometers in the indicated solvent at 298 K. Chemical shifts for proton and carbon spectra are reported on the delta scale in ppm and were referenced to residual solvent references or internal TMS reference.

1. A cyclic peptide, wherein the cyclic peptide has the following sequence:
[Formula 1]
[SEQ ID NO: 1]
C X₁ X₂ X₃ X₄ X₅
wherein:
i) C is selected from the group comprising or consisting of cys, h-cys and d-cys, optionally is cys;
ii) X₁ is selected from the group comprising or consisting of leu, lys and glu, optionally is lys or leu, optionally is lys;
iii) X₂ is: 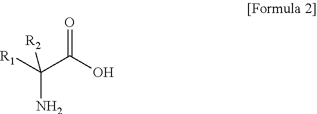
wherein:
R₁ is H or methyl;
R₂ is C₁-C₆ linear or branched alkyl, optionally substituted with —OC₁₋₃ alkyl
iv) X₃ is: 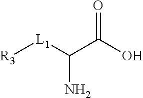
wherein:
L₁ is C₁-C₃ alkylene;
R₃ is selected from the group consisting of a C₃-C₆ cycloalkyl, a 5-6 membered ring and 8-10 membered bicyclic ring, wherein the rings are optionally substituted with phenyl, halogen, —OH, —O(C₁-C₃ alkyl), C₁-C₄ alkyl or C₁-C₄ haloalkyl; v) X₄ is: 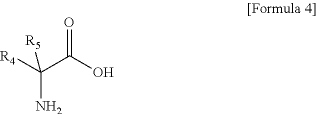
wherein:
R₄ is H or methyl;
R₅ is C₁-C₆ linear or branched alkyl, optionally substituted with —OC₁₋₃ alkyl;
vi) X₅ is: 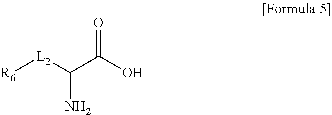
wherein:
L₂ is C₁-C₃ alkylene or a direct bond;
R₆ is selected from the group consisting of a C₃-C₆ cycloalkyl, 5-6 membered ring and 8-10 membered bicyclic ring, wherein the rings are optionally substituted with phenyl, halogen, —NO₂, —OH, —O(C₁-C₃ alkyl), C₁-C₄ alkyl or C₁-C₄ haloalkyl;
wherein:

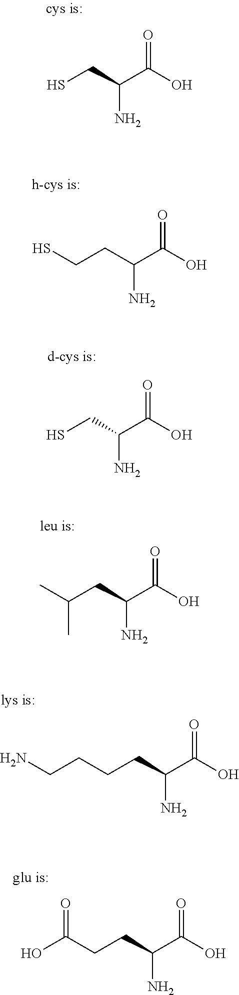

2. The cyclic peptide according to claim 1 wherein the peptide does not comprise the sequence: Cys leu leu phe val tyr [SEQ ID NO: 44].

3. The cyclic peptide according to claim 1 wherein the cyclic peptide comprises at least one of:
X₂ wherein:
(i) R₁ and R₂ are methyl (aib); or
(ii) R₁ is H and R₂ is selected from the group consisting of (S)-isopentyl (h-leu), (S)-n-propyl (n-val), (S)-methoxymethyl (ser(OMe)), and (S)-n-butyl (n-leu), preferably
(iii) R₁ is H and R₂ is selected from the group consisting of (S)-isopentyl (h-leu), (S)-n-propyl (n-val), (S)-methoxymethyl (ser(OMe)), and (S)-n-butyl (n-leu), more preferably;
(iv) R₁ is H and R₂ is (S)-n-butyl (n-leu); or
X₃ wherein:
(i) L₁ is (S)-methylene and R₃ is selected from the group consisting of cyclohexyl (Cha), 4-iodophenyl (phe(4-I)), 4-chlorophenyl (phe(4-Cl)), 4-bromophenyl (phe(4-Br)), 4-phenylphenyl (phe(4-Ph)), 4-trifluoromethylphenyl (phe(4-CF₃)) and 1-naphtyl (NaI), or
(ii) L₁ is (R)-methylene and R₃ is phenyl (d-Phe), or
(iii) L₁ is ethylene, R₃ is selected from the group consisting of phenyl (h-Phe) and 7-hydroxy-4-coumarinyl (Cou), preferably
(iv) L₁ is (S)-methylene and 0.3 is selected from the group consisting of 4-iodophenyl (phe(4-I)), 4-chlorophenyl (phe(4-Cl)), 4-bromophenyl (phe(4-Br)), 4-phenylphenyl (phe(4-Ph)), and 4-trifluoromethylphenyl (phe(4-CF3)), 1-naphtyl (NaI), or
(v) L₁ is (R)-methylene and R₃ is phenyl (d-Phe), or
(vi) L₁ is ethylene and R₃ is 7-hydroxy-4-coumarinyl (Cou), more preferably
(vii) L₁ is (S)-methylene and R₃ is 4-trifluoromethylphenyl (phe(4-CF₃)); or
X₄ wherein:
(i) R₄ and R₅ are methyl (aib),
(ii) R₄ is H and R₅ is selected from the group consisting of ethyl (abu), (S)-n-butyl (n-leu) and (S)-methoxymethyl (ser(OMe)), preferably
(iii) R₄ is H and R₅ is selected from (S)-n-butyl (n-leu) and (S)-methoxymethyl (ser(OMe)); or
X₅ wherein:
(i) L₂ is (S)-methylene and R₆ is selected from the group consisting of 4-pyrid-tyrl (4-Pal), cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
(ii) L₂ is (R)-methylene and R₆ is 4-hydroxyphenyl (d-tyr), or
(iii) L₂ is a direct bond and R₆ is phenyl (phg), or
(iv) L₂ is ethylene and R₆ is phenyl (h-phe), preferably
(v) L₂ is (S)-methylene and R₆ is selected from the group consisting of cyclohexyl (Cha), 4-phenylphenyl (phe(4-Ph)), 4-trifluoromethylphenyl (phe(4-CF₃)), 4-iodophenyl (phe(4-I)), 4-fluorophenyl (phe(4-F)), 4-chlorophenyl (phe(4-Cl)), 4-bromophenyl (phe(4-Br)), 4-nitrophenyl (phe(4-NO₂)), 1-naphtyl (NaI) and 4-methoxyphenyl (tyr(Me)), or
(vi) L₂ is (R)-methylene and R₆ is 4-hydroxyphenyl (d-tyr), or
(vii) L₂ is a direct bond and R₆ is phenyl (phg), or
(viii) L₂ is ethylene and R₆ is phenyl (h-phe), more preferably
(ix) L₂ is (S)-methylene and R₆ is selected from the group consisting of 4-iodophenyl (phe(4-I)), 4-fluorophenyl (phe(4-F)), 4-chlorophenyl (phe(4-Cl)), 4-bromophenyl (phe(4-Br)) and 4-nitrophenyl (phe(4-NO₂)), or
(x) L₂ is ethylene and R₆ is phenyl (h-phe), most preferably
(xi) L₂ is (S)-methylene and R₆ is 4-bromophenyl (phe(4-Br)).

4. The cyclic peptide according to claim 1 wherein R₄ is H and R₅ is (S,S)-2-butyl (ile).

5. The cyclic peptide according to claim 1 wherein where R₄ is H and R₅ is (S,S)-2-butyl (ile), then
X₂ selected from Aib, h-leu, n-val, Prop, or n-leu, optionally selected from hL, n-val, Ser(OMe) or n-leu, optionally n-leu; and
X₃ selected from cha, h-phe, phe(4-I), phe(4-Cl), d-phe, NaI, phe(4-Br), phe(4-Ph), phe(CF3) and Cou; optionally phe(4-I), phe(4-Cl), d-phe, NaI, phe(4-Br), phe(4-Ph), phe(CF3) and Cou; optionally phe(CF3); and
X₅ is selected from Pal, Cha, phe(4-Ph), d-tyr, Phg, phe(CF3), tyr(Me), phe(4-I), h-phe, phe(NO2), NaI, phe(4-F), phe(4-Cl), or phe(4-Br); optionally Cha, phe(4-Ph), d-tyr, Phg, phe(CF3), tyr(Me), phe(4-I), h-phe, phe(NO2), NaI, phe(4-F), phe(4-Cl), or phe(4-Br); optionally phe(4-I), h-phe, phe(NO2), NaI, phe(4-F), phe(4-Cl), or phe(4-Br); optionally phe(4-Br).

6. The cyclic peptide according to claim 1 wherein:
i) C is selected from the group comprising or consisting of cys, h-cys and d-cys, optionally is cys;
ii) X₁ is selected from the group comprising or consisting of leu, lys and glu, optionally is lys or leu, optionally is lys;
iii) X₂ is selected from the group comprising or consisting leu, val, aib, h-leu, n-val, ser(OMe), ile, n-leu, optionally selected from the group comprising or consisting of h-leu, n-val, ser(OMe), ile, n-leu, optionally selected from the group comprising or consisting of ser(OMe) or ile;
iv) X₃ is selected from the group comprising or consisting of phe, Cha and h-phe, phe(4-I), phe(4-Cl), d-phe, NaI, phe(4-Br), phe(4-Ph) and phe(4-CF₃), Cou; optionally selected from the group comprising or consisting of phe(4-I), phe(4-Cl), d-phe, NaI, phe(4-Br), phe(4-Ph) and phe(4-CF₃), Cou; optionally is phe(4-CF₃);
v) X₄ is selected from the group comprising or consisting val, aib, abu, n-leu, ser(OMe), leu, ile; optionally selected from the group comprising or consisting of n-leu, ser(OMe), leu, ile; optionally selected from the group comprising or consisting of leu, ile; optionally is ile; and
vi) X₅ is selected from the group comprising or consisting tyr, 4-Pal, Cha, phe(4-Ph), d-tyr, phg, phe(4-CF₃), phe, tyr(Me), phe(4-I), h-phe, phe(4-NO₂), Nal, phe(4-F), phe(4-Cl), phe(4-Br); optionally selected from the group comprising or consisting of Cha, phe(4-Ph), d-tyr, phg, phe(4-CF₃), phe, tyr(Me), phe(4-I), h-phe, phe(4-NO₂), Nal, phe(4-F), phe(4-Cl), phe(4-Br); optionally selected from the group comprising or consisting of phe(4-I), h-phe, phe(4-NO₂), Nal, phe(4-F), phe(4-Cl), phe(4-Br); optionally is phe(4-Br);
wherein:

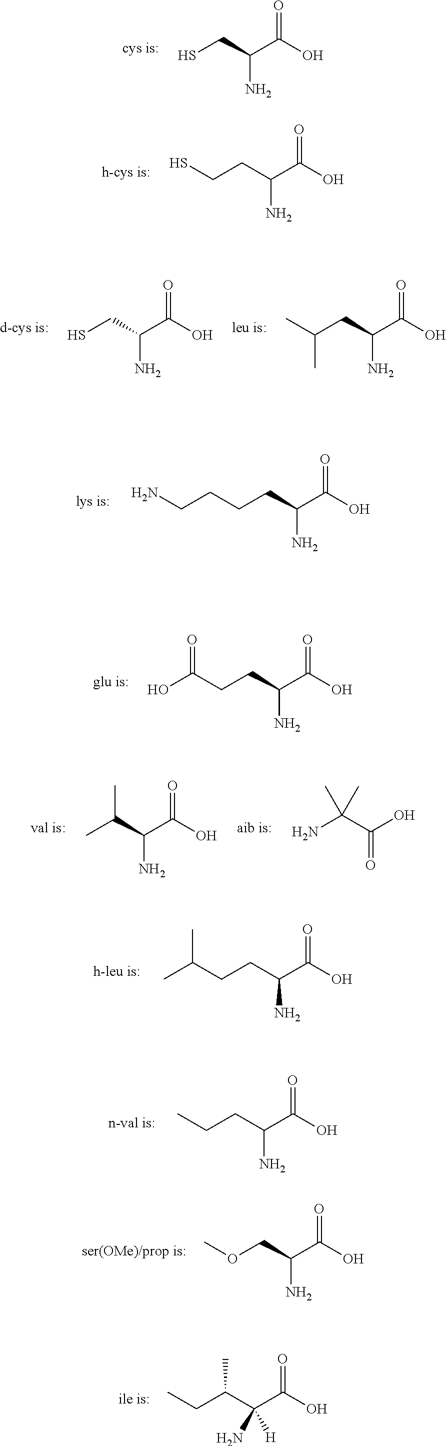

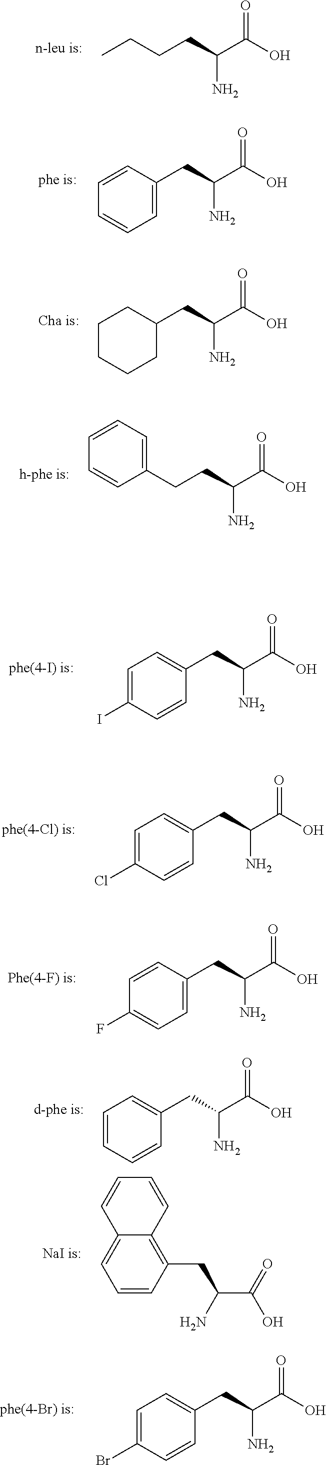

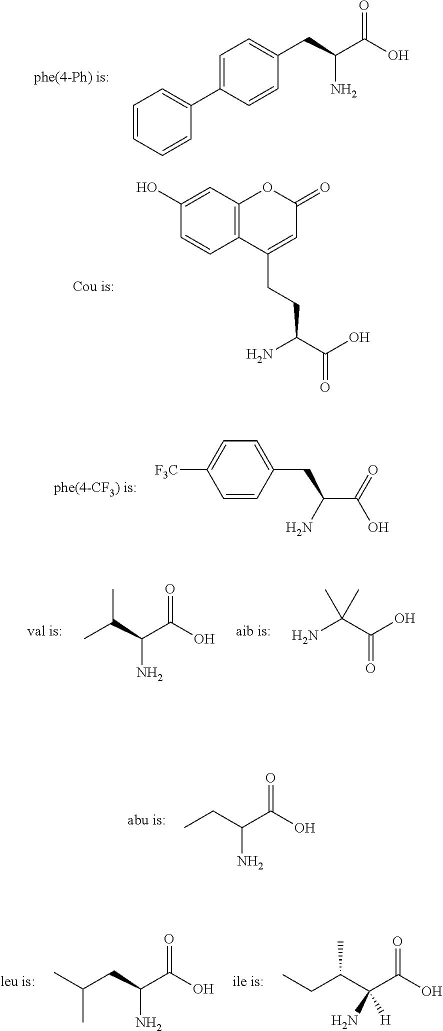

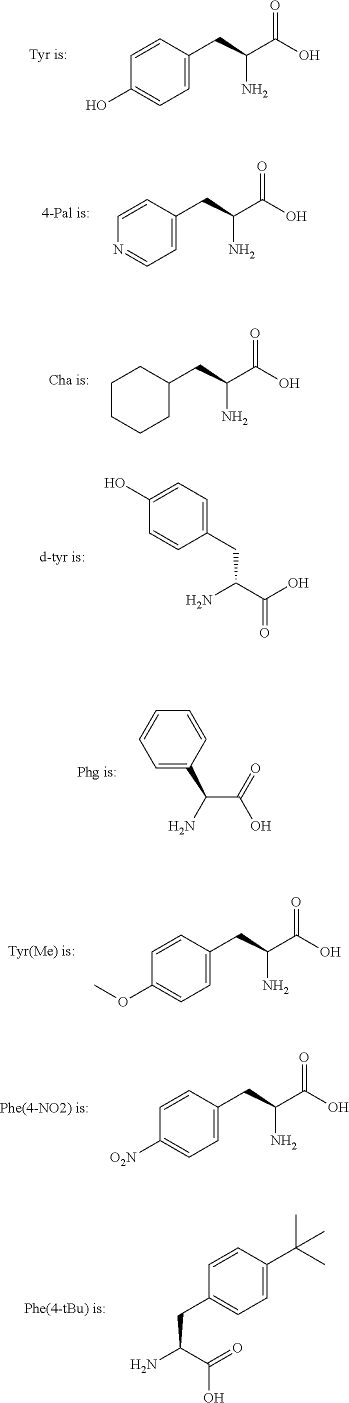

7. The cyclic peptide according to claim 1 wherein the cyclic peptide comprises at least one of:
X₂ selected from aib, h-leu, n-val, ser(OMe), n-leu, optionally selected from h-leu, n-val, ser(OMe), and n-leu, optionally n-leu; or
X₃ selected from Cha, h-phe, phe(4-I), phe(4-Cl), d-phe, NaI, phe(4-Br), phe(4-Ph), phe(4-CF₃), phe(4-tBu) and Cou, optionally selected from phe(4-I), phe(4-Cl), d-phe, NaI, phe(4-Br), phe(4-Ph), phe(4-CF₃), phe(4-tBu) and Cou, optionally is phe(4-CF₃); or
X₄ is selected from aib, abu, n-leu, n-val, or ser(OMe), optionally selected from n-leu or ser(OMe); or
X₅ is selected from 4-Pal, Cha, phe(4-Ph), d-tyr, phg, phe(4-CF₃), tyr(Me), phe(4-I), h-phe, phe(4-NO₂), Nal, phe(4-F), phe(4-Cl), phe(4-tBu) or phe(4-Br), optionally selected from Cha, phe(4-Ph), d-tyr, phg, phe(4-CF₃), tyr(Me), phe(4-I), h-phe, phe(4-NO₂), Nal, phe(4-F), phe(4-Cl), phe(4-tBu) and phe(4-Br), optionally selected from phe(4-I), h-phe, phe(4-NO₂), Nal, phe(4-F), phe(4-Cl), phe(4-Br), optionally is phe(4-Br).

8. The cyclic peptide according to claim 1 wherein where X₄ is ile, then:
X₂ is selected from aib, h-leu, n-val, ser(OMe), n-leu, optionally selected from h-leu, n-val, ser(OMe), and n-leu, optionally n-leu; or
X₃ is selected from Cha, h-phe, phe(4-I), phe(4-Cl), d-phe, NaI, phe(4-Br), phe(4-Ph), phe(4-CF₃) and Cou, optionally selected from phe(4-I), phe(4-Cl), d-phe, Nal, phe(4-Br), phe(4-Ph), phe(4-CF₃) and Cou, optionally is phe(4-CF₃); or
X₅ is selected from 4-Pal, Cha, phe(4-Ph), d-tyr, phg, phe(4-CF₃), tyr(Me), phe(4-I), h-phe, phe(4-NO₂), Nal, phe(4-F), phe(4-Cl) or phe(4-Br), optionally selected from Cha, phe(4-Ph), d-tyr, phg, phe(4-CF₃), tyr(Me), phe(4-I), h-phe, phe(4-NO₂), Nal, phe(4-F), phe(4-Cl) and phe(4-Br), optionally selected from phe(4-I), h-phe, phe(4-NO₂), Nal, phe(4-F), phe(4-Cl), phe(4-Br), optionally is phe(4-Br).

9. The cyclic peptide according to claim 1 wherein where X₄ is Ile, then
X₂ is selected from Aib, h-leu, n-val, Prop, or n-leu, optionally selected from h-leu, N-VAL, Prop or n-leu, optionally n-leu; and
X₃ is selected from ha, h-phe, phe(I), phe(4-Cl), d-phe, NaI, phe(4-Br), phe(4-Ph), phe(CF3) and Cou; optionally phe(I), phe(Cl), dphe, NaI, phe(Br), phe(4-Ph), phe(CF3) and Cou; optionally phe(CF3); and
X₄ is selected from Abu, Aib, n-leu, or Prop; optionally n-leu or Prop; and
X₅ is selected from Pal, Cha, phe(4-Ph), d-tyr, Phg, phe(CF3), tyr(Me), F(I), hF, phe(NO2), NaI, phe(F), phe(Cl), or phe(Br); optionally Cha, phe(4-Ph), d-tyr, Phg, phe(CF3), tyr(Me), F(I), hF, phe(NO2), NaI, phe(F), phe(Cl), or phe(Br); optionally F(I), hF, phe(NO2), NaI, phe(F), phe(Cl), or phe(Br); optionally phe(Br).

10. The cyclic peptide according to claim 1 wherein the peptide comprises, or consists of, one of the following sequences:
[SEQ ID NO: 2]
Cys leu leu Cha val tyr
[SEQ ID NO: 3]
Cys leu leu hphe val tyr
[SEQ ID NO: 4]
Cys leu leu phe(I) val tyr
[SEQ ID NO: 5]
Cys leu leu phe(Cl) val tyr
[SEQ ID NO: 6]
Cys leu leu dphe val tyr
[SEQ ID NO: 7]
Cys leu leu Nal val tyr
[SEQ ID NO: 8]
Cys leu leu phe(Br) val tyr
[SEQ ID NO: 9]
Cys leu leu phe(4-Ph) val tyr
[SEQ ID NO: 10]
Cys leu leu phe(CF3) val tyr
[SEQ ID NO: 11]
Cys leu leu cou val tyr
[SEQ ID NO: 56]
Cys leu leu phe(4-tBu) val tyr
optionally comprises or consists of one of the following sequences:
[SEQ ID NO: 4]
Cys leu leu phe(I) val tyr
[SEQ ID NO: 5]
Cys leu leu phe(Cl) val tyr
[SEQ ID NO: 6]
Cys leu leu dphe val tyr
[SEQ ID NO: 7]
Cys leu leu Nal val tyr
[SEQ ID NO: 8]
Cys leu leu phe(Br) val tyr
[SEQ ID NO: 9]
Cys leu leu phe(4-Ph) val tyr
[SEQ ID NO: 10]
Cys leu leu phe(CF3) val tyr
[SEQ ID NO: 39]
Cys leu leu cou val tyr
[SEQ ID NO: 56]
Cys leu leu phe(4-tBu) val tyr
optionally comprises or consists of the following sequence:
[SEQ ID NO: 10]
Cys leu leu phe(CF₃) val tyr
[SEQ ID NO: 56]
Cys leu leu phe(4-tBu) val tyr
optionally comprises or consists of the following sequence:
[SEQ ID NO: 10]
Cys leu leu phe(CF₃) val tyr.

11. The cyclic peptide according to claim 1 wherein the peptide comprises, or consists of, one of the following sequences:
[SEQ ID NO: 11]
Cys leu leu phe(CF₃) val Pal
[SEQ ID NO: 12]
Cys leu leu phe(CF₃) val Cha
[SEQ ID NO: 13]
Cys leu leu phe(CF₃) val phe(4-Ph)
[SEQ ID NO: 14]
Cys leu leu phe(CF₃) val d-tyr
[SEQ ID NO: 15]
Cys leu leu phe(CF₃) val Phg
[SEQ ID NO: 16]
Cys leu leu phe(CF3) val phe(4-CF3)
[SEQ ID NO: 17]
Cys leu leu phe(CF₃) val phe
[SEQ ID NO: 18]
Cys leu leu phe(CF₃) val tyr(Me)
[SEQ ID NO: 19]
Cys leu leu phe(CF₃) val phe(I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(F)
[SEQ ID NO: 24]
Cys leu leu phe(CF₃) val phe(Cl)
[SEQ ID NO: 25]
Cys leu leu phe(CF₃) val phe(Br)
[SEQ ID NO: 57]
Cys leu leu phe(CF₃) val phe(4-tBu)
optionally comprises or consists of one of the following sequences:
[SEQ ID NO: 12]
Cys leu leu phe(CF₃) val Cha
[SEQ ID NO: 13]
Cys leu leu phe(CF₃) val phe(4-Ph)
[SEQ ID NO: 14]
Cys leu leu phe(CF₃) val d-tyr
[SEQ ID NO: 15]
Cys leu leu phe(CF₃) val Phg
[SEQ ID NO: 16]
Cys leu leu phe(CF3) val phe(4-CF3)
[SEQ ID NO: 17]
Cys leu leu phe(CF₃) val phe
[SEQ ID NO: 18]
Cys leu leu phe(CF₃) val tyr(Me)
[SEQ ID NO: 19]
Cys leu leu phe(CF₃) val phe(I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(F)
[SEQ ID NO: 24]
Cys leu leu phe(CF₃) val phe(Cl)
[SEQ ID NO: 25]
Cys leu leu phe(CF₃) val phe(Br)
[SEQ ID NO: 57]
Cys leu leu phe(CF₃) val phe(4-tBu)
optionally comprises or consists of one of the following sequences:
[SEQ ID NO: 19]
Cys leu leu phe(CF₃) val phe(I)
[SEQ ID NO: 20]
Cys leu leu phe(CF₃) val h-phe
[SEQ ID NO: 21]
Cys leu leu phe(CF₃) val phe(NO₂)
[SEQ ID NO: 22]
Cys leu leu phe(CF₃) val Nal
[SEQ ID NO: 23]
Cys leu leu phe(CF₃) val phe(F)
[SEQ ID NO: 24]
Cys leu leu phe(CF₃) val phe(Cl)
[SEQ ID NO: 25]
Cys leu leu phe(CF₃) val phe(Br)
[SEQ ID NO: 57]
Cys leu leu phe(CF₃) val phe(4-tBu)
optionally comprises or consists of the following sequence:
[SEQ ID NO: 25]
Cys leu leu phe(CF₃) val phe(Br).

12. The cyclic peptide according to claim 1 wherein the peptide comprises, or consists of, one of the following sequences:
[SEQ ID NO: 26]
Cys leu leu phe(CF₃) Abu phe(4-Br)
[SEQ ID NO: 27]
Cys leu leu phe(CF₃) Aib phe(4-Br)
[SEQ ID NO: 28]
Cys leu leu phe(CF₃) n-leu phe(Br)
[SEQ ID NO: 29]
Cys leu leu phe(CF₃) Prop phe(Br)
[SEQ ID NO: 30]
Cys leu leu phe(CF₃) leu phe(4-Br)
[SEQ ID NO: 31]
Cys leu leu phe(CF₃) ile phe(Br)
[SEQ ID NO: 58]
Cys leu leu phe(CF₃) n-val phe(4-Br)
[SEQ ID NO: 59]
Cys leu leu phe(CF₃) h-leu phe(4-Br)
optionally comprises or consists of one of the following sequences:
[SEQ ID NO: 28]
Cys leu leu phe(CF₃) n-leu phe(Br)
[SEQ ID NO: 29]
Cys leu leu phe(CF₃) Prop phe(Br)
[SEQ ID NO: 30]
Cys leu leu phe(CF₃) leu phe(4-Br)
[SEQ ID NO: 31]
Cys leu leu phe(CF₃) ile phe(Br)
[SEQ ID NO: 59]
Cys leu leu phe(CF₃) h-leu phe(4-Br)
optionally comprises or consists of one of the following sequences:
[SEQ ID NO: 30]
Cys leu leu phe(CF₃) leu phe(4-Br)
[SEQ ID NO: 31]
Cys leu leu phe(CF₃) ile phe(Br)
[SEQ ID NO: 59]
Cys leu leu phe(CF₃) h-leu phe(4-Br)
optionally comprises or consists of the following sequence:
[SEQ ID NO: 31]
Cys leu leu phe(CF₃) ile phe(Br).

13. The cyclic peptide according to claim 1 wherein the peptide comprises, or consists of, one of the following sequences:
[SEQ ID NO: 32]
Cys leu val phe(CF₃) ile phe(Br)
[SEQ ID NO: 33]
Cys leu Aib phe(CF₃) ile phe(Br)
[SEQ ID NO: 34]
Cys leu h-leu phe(CF₃) ile phe(Br)
[SEQ ID NO: 35]
Cys leu n-val phe(CF₃) ile phe(Br)
[SEQ ID NO: 36]
Cys leu Prop phe(CF₃) ile phe(Br)
[SEQ ID NO: 37]
Cys leu ile phe(CF₃) ile phe(Br)
[SEQ ID NO: 38]
Cys leu n-leu phe(CFCF₃3) ile phe(Br);
optionally comprises or consists of one of the following sequences:
[SEQ ID NO: 34]
Cys leu h-leu phe(CF₃) ile phe(Br)
[SEQ ID NO: 35]
Cys leu n-val phe(CF₃) ile phe(Br)
[SEQ ID NO: 36]
Cys leu Prop phe(CF₃) ile phe(Br)
[SEQ ID NO: 37]
Cys leu ile phe(CF₃) ile phe(Br)
[SEQ ID NO: 38]
Cys leu n-leu phe(CFCF₃3) ile phe(Br)
optionally comprises or consists of the following sequence:
[SEQ ID NO: 38]
Cys leu n-leu phe(CFCF₃3) ile phe(Br)

14. The cyclic peptide according to claim 1 wherein the peptide comprises, or consists of, one of the following sequences:
Cys lys n-leu phe(CF₃) ile phe(Br)
Cys glu n-leu phe(CF₃) ile phe(Br).

15. The cyclic peptide according to claim 1 wherein the peptide comprises, or consists of, one of the following sequences:
[SEQ ID NO: 40]
h-cys leu leu phe val tyr
[SEQ ID NO: 41]
d-cys leu leu phe val tyr.

16. The cyclic peptide according to claim 1 wherein any one or more of C X₁ X₂ X₃ X₄ X₅ is a D amino acid.

17. The cyclic peptide according to claim 1 wherein any one or more of C X₁ X₂ X₃ X₄ X₅ is an L amino acid.

18. The cyclic peptide according to claim 1 wherein the cyclic peptide is capable of binding to HIF-1, optionally capable of binding to recombinantly expressed PAS-B domain of HIF-1α.

19. The cyclic peptide according to claim 1 wherein the cyclic peptide is capable of binding to recombinantly expressed PAS-B domain of HIF-1α with a Kd of:
less than 40 μM, 37 μM, 36 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2.5 μM, 2 μM, 1.5 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 40 μM, 0.2 μM and 37 μM, 0.3 μM and 36 μM, 0.4 μM and 35 μM, 0.5 μM and 30 μM, 0.6 μM and 25 μM, 0.7 μM and 20 μM, 0.8 μM and 18 μM, 0.9 μM and 16 μM, 1 μM and 15 μM, 2 μM and 14 μM, 3 μM and 13 μM, 4 μM and 12 μM, 5 μM and 11 μM, 6 μM and 10 μM, 7 μM and 9 μM,
optionally wherein the affinity is determined against the recombinantly expressed PAS-B domain of HIF-1α using microscale thermophoresis.

20. The cyclic peptide according to claim 1 wherein the cyclic peptide has a solubility profile of:
less than 10 C Log P, optionally less than 9.5, 9.0, 8.5, 8.0, 7.5, 7.0, 6.5, 6.0, 5.5, 5.0 C Log P.

21. The cyclic peptide according to claim 1, wherein the cyclic peptide is able to disrupt the interaction between recombinantly expressed PAS-B domains from HIF-1α-HIF-1β with an IC50 of:
less than 50 μM, optionally less than 45 μM, 40 M, 35 M, 30 M, 25 μM, 20 M, 15 M, 10 μM or less than 5 M.

22. The cyclic peptide according to claim 1, wherein the cyclic peptide is able to disrupt the interaction between recombinantly expressed HIF-1 and a HRE element in a section of DNA with an IC50 of:
less than 75 μM, optionally less than 70 μM, 65 M, 60 M, 55 M, 50 PM, 45 M, 40 M, 35 μM, 30 M, 25 M, 20 M, 15 μM, 10 M or less than 5 M.

23. The cyclic peptide according to claim 1 wherein the cyclic peptide prevents or reduces the hypoxia induced expression from a promoter that comprises one or more hypoxia-responsive elements under hypoxic conditions.

24. The cyclic peptide according to claim 1 wherein the cyclic peptide reduces the hypoxia induced expression from a promoter that comprises one or more hypoxia-responsive elements under hypoxic conditions to less than 90%, 80%, 70%, 60%, 50%, 40%, 30%, 20%0, 10%, 5%, 4%, 3%, 2%, 1% of the expression obtained in the absence of the cyclic peptide.

25. A polynucleotide comprising or consisting of a sequence that encodes a cyclic peptide according to claim 1.

26. A polynucleotide comprising or consisting of a sequence that encodes an N-terminal intein fragment, followed by a sequence encoding a cyclic polypeptide according to claim 1, followed by a sequence encoding a C-terminal intein fragment.

27. A cell comprising the polynucleotide according to claim 25.

28. A pharmaceutical composition comprising one or more of the cyclic peptides according to claim 1, and/or a polynucleotide comprising or consisting of a sequence that encodes a cyclic peptide according to claim 1 and/or a cell comprising a polynucleotide comprising or consisting of a sequence that encodes a cyclic peptide according to claim 1, optionally wherein the pharmaceutical composition is formulated with one or more further therapeutic agents, optionally one or more further anti-cancer therapeutic agents.

29. (canceled)

30. (canceled)

31. (canceled)

32. (canceled)

33. (canceled)

34. (canceled)

35. A method for the treatment or prevention of a disease, wherein the method comprises:
(a) administration of one or more cyclic peptides according to claim 1;
(b) a polynucleotide comprising or consisting of a sequence that encodes a cyclic peptide according to claim 1 and/or
(c) a cell comprising a polynucleotide comprising or consisting of a sequence that encodes a cyclic peptide according to claim 1; or
(d) a pharmaceutical comprising one or more of the cyclic peptides according to claim 1, a polynucleotide comprising or consisting of a sequence that encodes a cyclic peptide according to claim 1, and/or a cell comprising a polynucleotide comprising or consisting of a sequence that encodes a cyclic peptide according to claim 1, optionally wherein the pharmaceutical composition is formulated with one or more further therapeutic agents, optionally one or more further anti-cancer therapeutic agents.

36. The method of claim 35, wherein the disease, disorder or condition is a disease, disorder or condition
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

37. (canceled)

38. (canceled)

39. (canceled)

40. A fluorescent probe wherein the fluorescent probe is a cyclic peptide according to claim 1 wherein X₃ is Cou.

41. The fluorescent probe according to claim 40 wherein the cyclic peptide has a sequence of: Cys leu leu cou val tyr [SEQ ID NO: 11].

42. A kit comprising one or more of:
a cyclic peptide according to claim 1;
a polynucleotide comprising or consisting of a sequence that encodes a cyclic peptide according to claim 1 according to claim 21;
a cell comprising a polypeptide comprising or consisting of a sequence that encodes a cyclic peptide according to claim 1;
a pharmaceutical composition comprising one or more of the cyclic peptides according to claim 1, a polynucleotide comprising or consisting of a sequence that encodes a cyclic peptide according to claim 1, and/or a cell comprising a polynucleotide comprising or consisting of a sequence that encodes a cyclic peptide according to claim 1, optionally wherein the pharmaceutical composition is formulated with one or more further therapeutic agents, optionally one or more further anti-cancer therapeutic agents; or
a fluorescent probe, wherein the fluorescent probe is a cyclic peptide according to claim 1, wherein X₃ is Cou.

43. the method of claim 35, wherein the pharmaceutical composition is administered as part of a combination therapy, optionally wherein the cyclic peptide, polynucleotide or pharmaceutical composition is administered prior to, subsequent to, or simultaneously with one or more further therapeutic agents.

44. The method of claim 35, wherein the diseases is cancer, wherein the cancer is selected from the group comprising or consisting of:
acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholangiocarcinoma), Bladder cancer, Bone tumor, osteosarcoma/malignant fibrous histiocytoma, Brainstem glioma, Brain cancer, cerebellar astrocytoma, cerebral astrocytoma/malignant glioma, epend-tyrmoma, medulloblastoma, supratentorial primitive neuroectodermal tumors, Breast cancer, Bronchial adenomas/carcinoids, Burkitt's lymphoma, Carcinoid tumor, childhood, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Epend-tyrmoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemia, acute lymphoblastic (also called acute lymphocytic leukaemia), acute myeloid (also called acute myelogenous leukemia), chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelod-tyrsplastic syndromes Myelod-tyrsplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma, Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma—see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).


SEQUENCE LISTING (59 sequences)

SEQ ID NO: 1 - skipped

SEQ ID NO: 2 - protein, 6 aa, synthetic construct
  MOD_RES 4: note = X is cyclohexylalanine
CLLXVY

SEQ ID NO: 3 - protein, 6 aa, synthetic construct
  MOD_RES 4: note = X is homophenylalanine
CLLFVY

SEQ ID NO: 4 - protein, 6 aa, synthetic construct
  MOD_RES 4: note = X is 4-iodophenylalanine
CLLXVY

SEQ ID NO: 5 - protein, 6 aa, synthetic construct
  MOD_RES 4: note = X is 4-chlorophenylalanine
CLLXVY

SEQ ID NO: 6 - protein, 6 aa, synthetic construct
  MOD_RES 4: note = X is D-phenylalanine
CLLXVY

SEQ ID NO: 7 - protein, 6 aa, synthetic construct
  MOD_RES 4: note = X is 3-(1-Naphthyl)-alanine
CLLXVY

SEQ ID NO: 8 - protein, 6 aa, synthetic construct
  MOD_RES 4: note = X is 4-bromophenylalanine
CLLXVY

SEQ ID NO: 9 - protein, 6 aa, synthetic construct
  MOD_RES 4: note = X is p-phenyl-phenylalanine
CLLXVY

SEQ ID NO: 10 - protein, 6 aa, synthetic construct
  MOD_RES 4: note = X is 4-trifluoromethylphenylalanine
CLLXVY

SEQ ID NO: 11 - protein, 6 aa, synthetic construct
  MOD_RES 4: note = X is 4-trifluoromethylphenylalanine
  MOD_RES 6: note = X is 4-pyridylalanine
CLLXVX

SEQ ID NO: 12 - protein, 6 aa, synthetic construct
  MOD_RES 4: note = X is 4-trifluoromethylphenylalanine
  MOD_RES 6: note = X is cyclohexylalanine
CLLXVX

SEQ ID NO: 13 - protein, 6 aa, synthetic construct
  MOD_RES 4: note = X is 4-trifluoromethylphenylalanine
  MOD_RES 6: note = X is p-phenyl-phenylalanine
CLLXVX

SEQ ID NO: 14 - protein, 6 aa, synthetic construct
  MOD_RES 4: note = X is 4-trifluoromethylphenylalanine
  MOD_RES 6: note = X is D-tyrosine
CLLXVX

SEQ ID NO: 15 - protein, 6 aa, synthetic construct
  MOD_RES 4: note = X is 4-trifluoromethylphenylalanine
  MOD_RES 6: note = X is phenylglycine
CLLXVX

SEQ ID NO: 16 - protein, 6 aa, synthetic construct
  MOD_RES 4: note = X is 4-trifluoromethylphenylalanine
  MOD_RES 6: note = X is 4-trifluoromethylphenylalanine
CLLXVX

SEQ ID NO: 17 - protein, 6 aa, synthetic construct
  MOD_RES 4: note = X is 4-trifluoromethylphenylalanine
CLLXVF

SEQ ID NO: 18 - protein, 6 aa, synthetic construct
  MOD_RES 4: note = X is 4-trifluoromethylphenylalanine
  MOD_RES 6: note = X is O-methyltyrosine
CLLXVX

SEQ ID NO: 19 - protein, 6 aa, synthetic construct
  MOD_RES 4: note = X is 4-trifluoromethylphenylalanine
  MOD_RES 6: note = X is 4-iodophenylalanine
CLLXVX

SEQ ID NO: 20 - protein, 6 aa, synthetic construct
  MOD_RES 4: note = X is 4-trifluoromethylphenylalanine
  MOD_RES 6: note = X is homophenylalanine
CLLXVX

SEQ ID NO: 21 - protein, 6 aa, synthetic construct
  MOD_RES 4: note = X is 4-trifluoromethylphenylalanine
  MOD_RES 6: note = X is 4-nitrophenylalanine
CLLXVX

SEQ ID NO: 22 - protein, 6 aa, synthetic construct
  MOD_RES 4: note = X is 4-trifluoromethylphenylalanine
  MOD_RES 6: note = X is 3-(1-Naphthyl)-alanine
CLLXVX

SEQ ID NO: 23 - protein, 6 aa, synthetic construct
  MOD_RES 4: note = X is 4-trifluoromethylphenylalanine
  MOD_RES 6: note = X is 4-fluorophenylalanine
CLLXVX

SEQ ID NO: 24 - protein, 6 aa, synthetic construct
  MOD_RES 4: note = X is 4-trifluoromethylphenylalanine
  MOD_RES 6: note = X is 4-chlorophenylalanine
CLLXVX

SEQ ID NO: 25 - protein, 6 aa, synthetic construct
  MOD_RES 4: note = X is 4-trifluoromethylphenylalanine
  MOD_RES 6: note = X is 4-bromophenylalanine
CLLXVX

SEQ ID NO: 26-29 - skipped (4 entries)

SEQ ID NO: 30 - protein, 6 aa, synthetic construct
  MOD_RES 4: note = X is 4-trifluoromethylphenylalanine
  MOD_RES 6: note = X is 4-bromophenylalanine
CLLXLX

SEQ ID NO: 31 - protein, 6 aa, synthetic construct
  MOD_RES 4: note = X is 4-trifluoromethylphenylalanine
  MOD_RES 6: note = X is 4-bromophenylalanine
CLLXIX

SEQ ID NO: 32 - protein, 6 aa, synthetic construct
  MOD_RES 4: note = X is 4-trifluoromethylphenylalanine
  MOD_RES 6: note = X is 4-bromophenylalanine
CLVXIX

SEQ ID NO: 33-36 - skipped (4 entries)

SEQ ID NO: 37 - protein, 6 aa, synthetic construct
  MOD_RES 4: note = X is 4-trifluoromethylphenylalanine
  MOD_RES 6: note = X is 4-bromophenylalanine
CLIXIX

SEQ ID NO: 38 - skipped

SEQ ID NO: 39 - protein, 6 aa, synthetic construct
  MOD_RES 4: note = X is Courmarin
CLLXVY

SEQ ID NO: 40-41 - skipped (2 entries)

SEQ ID NO: 42 - protein, 6 aa, synthetic construct
  MOD_RES 1: note = X is homocysteine
XLLFVY

SEQ ID NO: 43 - protein, 6 aa, synthetic construct
  MOD_RES 1: note = X is D-cysteine
XLLFVY

SEQ ID NO: 44 - protein, 6 aa, synthetic construct
CLLFVY

SEQ ID NO: 45 - protein, 6 aa, synthetic construct
SLLFVY

SEQ ID NO: 46 - protein, 6 aa, synthetic construct
TLLFVY

SEQ ID NO: 47 - protein, 6 aa, synthetic construct
MLLFVY

SEQ ID NO: 48 - protein, 6 aa, synthetic construct
WLLFVY

SEQ ID NO: 49 - protein, 6 aa, synthetic construct
ALLFVY

SEQ ID NO: 50 - protein, 6 aa, synthetic construct
  MOD_RES 4: note = X is O-methyltyrosine
CLLXVY

SEQ ID NO: 51 - protein, 6 aa, synthetic construct
CLLYVY

SEQ ID NO: 52 - protein, 6 aa, synthetic construct
  MOD_RES 4: note = X is phenylglycine
CLLXVY

SEQ ID NO: 53 - protein, 6 aa, synthetic construct
  MOD_RES 4: note = X is 4-pyridylalanine
CLLXVY

SEQ ID NO: 54 - protein, 6 aa, synthetic construct
  MOD_RES 4: note = X is 4-nitrophenylalanine
CLLXVY

SEQ ID NO: 55 - protein, 6 aa, synthetic construct
  MOD_RES 4: note = X is 4-fluorophenylalanine
CLLXVY

SEQ ID NO: 56 - protein, 6 aa, synthetic construct
  MOD_RES 4: note = X is 4-tert-butyl-phenylalanine
CLLXVY

SEQ ID NO: 57 - protein, 6 aa, synthetic construct
  MOD_RES 4: note = X is 4-trifluoromethylphenylalanine
  MOD_RES 6: note = X is 4-tert-butyl-phenylalanine
CLLXVX

SEQ ID NO: 58-59 - skipped (2 entries)